(19) United States
(12) Patent Application Publication
(10) Pub. No.: US 2015/0065810 A1
(43) Pub. Date: Mar. 5, 2015

(54) Frontal Sinus Recess Dilator

(71) Applicant: SinuSys Corporation, Palo Alto, CA (US)

(72) Inventors: David E. Edgren, Los Altos, CA (US); William Jason Fox, San Carlos, CA (US); Jerome E. Hester, Menlo Park, CA (US); Thomas A. Schreck, Portola Valley, CA (US); Curtis Leslie Rieser, Aptos, CA (US)

(21) Appl. No.: 14/470,731

(22) Filed: Aug. 27, 2014

Related U.S. Application Data
(60) Provisional application No. 61/871,180, filed on Aug. 28, 2013.

(51) Int. Cl. A61M 29/02; A61B 5/06

(52) U.S. Cl. CPC A61M 29/02; A61B 5/065; A61M 2029/025
USPC 600/249; 606/199; 606/196

(57) ABSTRACT

Dilators and methods for dilating a stenotic recess of a frontal sinus in a subject using minimally invasive insertion procedures are provided. Also provided are devices and methods for inserting said dilator into a stenotic opening of a paranasal sinus in a subject. The dilators, devices and methods can be used to treat sinusitis and other nasal and/or sinus disorders.

### CROSS-REFERENCE TO RELATED APPLICATION

This application claims priority pursuant to 35 U.S.C. §119(e) to the filing date of U.S. Provisional Application No. 61/871,180, filed Aug. 28, 2013, the disclosure of which is incorporated herein by reference.

### INTRODUCTION

The bones in the skull and face contain a series of air-filled cavities known as paranasal sinuses that are connected by passageways. The paranasal sinuses include frontal sinuses, sphenoid sinuses and maxillary sinuses. The paranasal sinuses are lined with mucus-producing epithelial tissue and are in communication with the nasal cavity. Normally, mucus produced by the epithelial tissue slowly drains out of each sinus through an opening known as an ostium. If the epithelial tissue of one of these passageways becomes inflamed for any reason, the cavities which drain through that passageway can become blocked. This blockage can be periodic (resulting in episodes of pain) or chronic. This interference with drainage of mucus (e.g., occlusion of a sinus ostium) can result in mucosal congestion within the paranasal sinuses. Chronic mucosal congestion of the sinuses can cause damage to the epithelium that lines the sinus with subsequent decreased oxygen tension and microbial growth (e.g., a sinus infection).

The term “sinusitis” refers generally to any inflammation or infection of the paranasal sinuses caused by bacteria, viruses, fungi (molds), allergies or combinations thereof. It has been estimated that chronic sinusitis (e.g., lasting more than 3 months) results in 18 million to 22 million physician office visits per year in the United States. Patients who suffer from sinusitis typically experience at least some of the following symptoms: headaches or facial pain, nasal congestion or post-nasal drainage, difficulty breathing through one or both nostrils, bad breath and/or pain in the upper teeth. Thus, one of the ways to treat sinusitis is by restoring the lost mucus flow and ventilation.

### SUMMARY

Devices and methods that are adapted to dilate a stenotic opening of a paranasal sinus of a subject patient are provided. The devices and methods can be used to treat sinusitis and other nasal and/or sinus disorders.

Aspects of the present disclosure include a dilator for dilating a stenotic recess of a frontal sinus in a subject is provided. The dilator includes a self-expanding member configured to radially expand from a non-expanded configuration to an expanded configuration, the non-expanded configuration being sized to be positioned within the stenotic recess. The dilator has a radially-expandable working length, the working length having (i) a plurality of non-bendable segments, and (ii) one or more bendable segments positioned between adjacent non-bendable segments. The dilator is malleable, meaning that it is flexible, and once flexed, it bends and substantially retains its bent shape. By “plurality” is meant 2 or more. The dilators have a malleability of 30 Newtons or less, as measured using the measurement methods and apparatus described herein.

In certain embodiments, the dilator has a malleability of 5 Newtons or more. In other embodiments, the dilator has a malleability of 20 Newtons or more. In still other embodiments, the dilator has a malleability of 10 to 20 Newtons.

In certain embodiments, the dilator has a malleability of 15 Newtons or more. In other embodiments, the dilator has a malleability of 20 Newtons or more. In still other embodiments, the dilator has a malleability of 20 to 25 Newtons.

In certain embodiments, the dilator includes a malleable axial member having a plurality of solid osmotic tablets disposed thereon and spaced apart from one another, and an elastic semipermeable membrane disposed over the tablets. By “plurality” is meant 2 or more. The malleable axial member can be selected from (i) a rod, (ii) a tube, (iii) a coil, and (iv) a wire.

In certain embodiments, the axial member is radially non-compressible and composed of a malleable metal or polymer. The malleable metal can be selected from aluminum, silver, gold, platinum, titanium, copper and annealed stainless steel. The malleable polymer can be selected from cross-linked polybutadiene, polyvinyl acetate, ethylene vinyl acetate and ethyl acrylate methyl methacrylate copolymer.

In certain embodiments, the dilator includes elastic spacers disposed on the axial member, the spacers being positioned between adjacent osmotic tablets.

In certain embodiments, the dilator has from 3 to 8 of the bendable segments and from 2 to 7 of the non-bendable segments. In certain embodiments, the dilator has from 5 to 7 of the bendable segments and from 4 to 6 of the non-bendable segments.

In certain embodiments, the dilator has a working length of 12 to 24 mm.

In certain embodiments, the dilator has a working length of 13 to 16 mm.

In certain embodiments, the dilator has an overall length that is greater than the working length.

In certain embodiments, each of the bendable segments has a length of 0.5 mm or more.

In certain embodiments, each of the bendable segments has a length of 1 to 1.5 mm.

In certain embodiments, each of the non-bendable segments has a length of 4 mm or less.

In certain embodiments, each of the non-bendable segments has a length of 2 to 3 mm.

In certain embodiments, the dilator includes an elongated flexible portion extending from a distal end of the dilator. In certain embodiments, the elongated flexible portion includes a spring.

Aspects of the present disclosure also include a kit that includes a frontal sinus dilator as described herein, and a device for inserting the dilator into a frontal sinus recess.

In certain embodiments, the kit includes two or more dilators.

Aspects of the present disclosure also include a device for inserting an osmotically-driven sinus dilator into a stenotic opening of a paranasal sinus in a subject, where the dilator is flexible and/or bendable along its length. The device includes a handle including a guidewire actuator and a dilator actuator, a tube having a proximal end coupled to the handle, a distal end adapted to be positioned adjacent to the stenotic sinus opening, and an interior cavity extending from the proximal end to the distal end. The interior cavity is sized to fit the dilator therein. The device also includes a guidewire extending through the interior cavity, the guidewire being displaceable (e.g., distally displaceable) through or adjacent to the dilator upon activation of the guidewire actuator. The device also includes an arm extending within the interior cavity of the tube, the arm being displaceable (e.g., distally displaceable) upon activation of the dilator actuator for deploying the dilator from the interior cavity.

In certain embodiments, the dilator includes an element configured to slide over the guidewire. In some instances, the element is an axial passageway through the dilator, the guidewire extending through the axial passageway when the dilator is positioned in the tube. In some instances, the element is a loop attached to a distal end of the dilator.

In certain embodiments, the distal end of the tube is angled. When the device is used to place an osmotically-driven dilator into a frontal sinus recess, the angle is from 50° to 90°. In some instances, the angle is 70°. When the device is used to place an osmotically-driven dilator into a maxillary sinus opening, the angle is from 90° to 140°. In some instances, the angle is 110°. When the device is used to place an osmotically-driven dilator into a sphenoid sinus opening, the angle is from 1° to 30°. In some instances, the angle is 15°.

In certain embodiments, activation of the guidewire actuator causes the guidewire to be displaced distally beyond the distal end of the tube.

In certain embodiments, activation of the dilator actuator causes the arm be displaced distally to push the dilator out of the distal end of the tube.

In certain embodiments, the dilator has a radially extending anchor and the distal end of the tube has a slot that accommodates the anchor while the dilator is positioned in the interior cavity.

In certain embodiments, the arm has a bore through which the guidewire extends.

In certain embodiments, the device includes a light source or a light-transmitting conduit for illuminating the nasal cavity and/or the sinus opening during dilator insertion. In certain embodiments, the guidewire is the light transmitting conduit.

Aspects of the present disclosure also include an assembly that includes the insertion device as described herein and an osmotically-driven sinus dilator positioned in the interior cavity.

Aspects of the present disclosure also include a method of dilating a stenotic opening of a paranasal sinus in a subject. The method includes placing an osmotically-driven sinus dilator in the interior cavity of an insertion device as described herein, inserting the tube of the insertion device into a nasal cavity of the subject, positioning the distal end of the tube adjacent the stenotic opening, activating the guidewire actuator causing the guidewire to extend beyond the distal end of the tube and into and/or through the sinus opening, activating the dilator actuator causing the dilator to be distally displaced along the extended guidewire and into the sinus opening, and removing the insertion device from the nasal cavity of the subject while leaving the dilator in place in the stenotic opening. In certain embodiments, once left in place in the sinus opening, the dilator dilates by way of osmosis.

In certain embodiments, the dilator includes an element configured to slide over the guidewire, and activating the dilator actuator causes the dilator to slide over the extended guidewire.

In certain embodiments, activating the dilator actuator causes the dilator to be displaced distally adjacent to the extended guidewire.

In certain embodiments, the method includes removing the dilator from the sinus opening after the dilator has dilated.

In certain embodiments, the sinus opening is a maxillary sinus opening. In other embodiments, the sinus opening is a frontal sinus recess. In other embodiments, the sinus opening is a sphenoid sinus opening.

In certain embodiments, the method includes illuminating the nasal cavity and/or the sinus opening during placement of the dilator into the stenotic opening.

### BRIEF DESCRIPTION OF THE FIGURES

FIG. 1 is a partial cutaway view of a human head showing the positions of the frontal sinuses (FS) and the maxillary sinuses (MS).

FIG. 2 is a sectional view through the right nasal cavity of a human head showing the positions of the nostril opening (NO), the nasal cavity (NC), the frontal sinus (FS) and the ethmoid air cells (EAC).

FIG. 3 is a sectional view through the right nasal cavity of a human head showing the positions of the nasal cavity (NC) and the frontal sinus (FS), wherein a dilator insertion device is positioned in the nasal cavity (NC).

FIG. 4 is a sectional view through the right nasal cavity of a human head showing the positions of the nasal cavity (NC) and the frontal sinus (FS), wherein a dilator is being inserted into the right frontal recess.

FIG. 5 is a sectional view through the right nasal cavity of a human head showing an expanded dilator after insertion into the right frontal recess and the insertion device removed.

FIG. 6 is a sectional view through the right nasal cavity of a human head showing an expanded frontal recess after removal of the dilator.

FIG. 7 is a side view of a frontal sinus recess dilator in a non-expanded configuration according to embodiments of the present disclosure.

FIG. 8 is a side view of a frontal sinus recess dilator in a non-expanded configuration, having two bends along its length, according to embodiments of the present disclosure.

FIG. 9 is an end view of the dilator shown in FIGS. 7 and 8 according to embodiments of the present disclosure.

FIG. 10 is a cross sectional view of the dilator shown in FIG. 9, along line 10-10 according to embodiments of the present disclosure.

FIG. 11 is a side view of the dilator shown in FIGS. 7 through 10, in an expanded configuration, according to embodiments of the present disclosure.

FIG. 12 is a sectional view of the dilator shown in FIG. 11 according to embodiments of the present disclosure.

FIG. 13 is a side view of a dilator insertion device according to embodiments of the present disclosure.

FIG. 14 is a side view of the dilator insertion device shown in FIG. 13 with a guidewire actuator activated according to embodiments of the present disclosure.

FIG. 15 is a side view of the dilator insertion device shown in FIG. 13 with a dilator deployment actuator activated according to embodiments of the present disclosure.

FIG. 16 is a side view of the dilator insertion device shown in FIG. 13 with a dilator deployment actuator activated according to embodiments of the present disclosure.

FIG. 17 is a sectional view of the distal end of the dilator insertion device shown in FIG. 13 according to embodiments of the present disclosure.

FIG. 18 is a sectional view of the distal end of the dilator insertion device shown in FIG. 14 according to embodiments of the present disclosure.

FIG. 19 is a sectional view of the distal end of the dilator insertion device shown in FIG. 15 according to embodiments of the present disclosure.

FIG. 20 is a sectional view of the distal end of the dilator insertion device shown in FIG. 16 according to embodiments of the present disclosure.

FIG. 21 is a front view of a holding fixture and bending probe used for measuring the malleability of tubes according to embodiments of the present disclosure.

FIG. 22 is an end view of the holding fixture and bending probe shown in FIG. 21, according to embodiments of the present disclosure.

FIG. 23 is a graph of load versus travel for certain non-annealed tubes, with the inflection point on the curve representing the malleability of the tubes, according to embodiments of the present disclosure.

FIG. 24 is a graph of load versus travel for tubes annealed at 1650° F., according to embodiments of the present disclosure.

FIG. 25 is a graph of load versus travel for tubes annealed at 1750° F., according to embodiments of the present disclosure.

FIG. 26 is a graph of load versus travel for tubes annealed at 1850° F., according to embodiments of the present disclosure.

FIG. 27 is a graph of load versus travel for tubes annealed at 1925° F., according to embodiments of the present disclosure.

FIG. 28 is a graph of load versus travel for tubes annealed at 2025° F., according to embodiments of the present disclosure.

FIG. 29 is a graph of dilator weight gain versus time in water for a dilator according to embodiments of the present disclosure.

FIG. 30 is a graph of dilator diameter versus time in water for a dilator according to embodiments of the present disclosure.

FIG. 31 is a graph of dilator weight gain versus time in water for a dilator according to embodiments of the present disclosure.

FIG. 32 is a graph of dilator diameter versus time in water for a dilator according to embodiments of the present disclosure.

FIG. 33 is an exploded perspective view of portions of a dilator and a fixture used to assemble a dilator according to embodiments of the present disclosure.

FIGS. 34a and 34b are each side cross sectional views of the dilator and fixture shown in FIG. 33 showing assembly of a dilator according to embodiments of the present disclosure.

FIGS. 35a and 35b are each side cross sectional views of the dilator and fixture shown in FIG. 33 showing assembly of a dilator according to embodiments of the present disclosure.

FIGS. 36a and 36b are each side cross sectional views of the assembly of a dilator with a double-layer outer membrane according to embodiments of the present disclosure. FIG. 36c shows an enlargement of a portion of FIG. 36b.

FIG. 37 shows an exploded perspective view drawing of a dilator being assembled according to embodiments of the present disclosure.

FIG. 38 is a side cross sectional view of a dilator according to embodiments of the present disclosure.

FIG. 39 is a perspective view of a fixture used for thermal bonding during the assembly of a dilator according to embodiments of the present disclosure.

FIG. 40 is a front perspective view of a holding fixture and bending probe used for measuring the malleability of a dilator according to embodiments of the present disclosure.

FIG. 41 is an end perspective view of the holding fixture and bending probe shown in FIG. 40, according to embodiments of the present disclosure.

FIG. 42 is a graph of load (N) versus displacement (mm) for a dilator, representing the malleability of the dilator, according to embodiments of the present disclosure.

FIG. 43 is an end view of a frontal sinus recess dilator according to embodiments of the present disclosure.

FIG. 44 is a cross sectional view of the dilator shown in FIG. 43, along line 44-44 according to embodiments of the present disclosure.

FIG. 45 is a side view of the dilator shown in FIGS. 43 and 44, in an expanded configuration, according to embodiments of the present disclosure.

FIG. 46 is a sectional view of the dilator shown in FIG. 45 according to embodiments of the present disclosure.

FIG. 47 is a perspective view of a frontal sinus recess dilator insertion device according to embodiments of the present disclosure.

FIG. 48 is a side view of the dilator insertion device shown in FIG. 47 according to embodiments of the present disclosure.

FIG. 49 is a side view of the dilator insertion device shown in FIGS. 47 and 48 with a lighted wire actuator activated according to embodiments of the present disclosure.

FIG. 50 is a side view of the dilator insertion device shown in FIGS. 47 through 49 with a dilator deployment actuator activated according to embodiments of the present disclosure.

FIG. 51 is a side view of the dilator insertion device shown in FIG. 50, with portions thereof removed for ease of illustration, according to embodiments of the present disclosure.

FIG. 52 is a perspective view of the device shown in FIG. 51 according to embodiments of the present disclosure.

FIG. 53 is an end view of the device shown in FIGS. 50 through 52 according to embodiments of the present disclosure.

FIG. 54 is a cross sectional view of the device shown in FIG. 53, along line 54-54 according to embodiments of the present disclosure.

FIG. 55 is an expanded sectional view of the distal end of the insertion device shown in FIG. 54 and the dilation device 300 according to embodiments of the present disclosure.

FIG. 56 is an end view of a frontal sinus recess dilator according to embodiments of the present disclosure.

FIG. 57 is a cross sectional view of the dilator shown in FIG. 56, along line 57-57 according to embodiments of the present disclosure.

FIG. 58 is a perspective, partially cut away view of the distal end of a dilator and insertion device constructed according to according to embodiments of the present disclosure.

FIG. 59 is a side, partially cut away view of the dilator and device shown in FIG. 58 according to embodiments of the present disclosure.

FIG. 60 is a side view of the dilator and device shown in FIGS. 58 and 59 with the dilator deployed according to embodiments of the present disclosure.

Before embodiments of the present disclosure are described in greater detail, it is to be understood that these embodiments are not limited to the particular aspects described, and as such may, of course, vary. It is also to be understood that the terminology used herein is for the purpose of describing particular embodiments only, and is not intended to be limiting, since the scope of the embodiments is embodied by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within embodiments of the present disclosure. The upper and lower limits of these smaller ranges may independently be included in the smaller ranges and are also encompassed within the embodiments, subject to any specifically excluded limit in the stated range. Where the stated range includes one or both of the limits, ranges excluding either or both of those included limits are also included in the embodiments.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present embodiments, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, it will be readily apparent to one of ordinary skill in the art in light of the teachings herein that certain changes and modifications may be made thereto without departing from the spirit and scope of the appended claims. Any recited method can be carried out in the order of events recited or in any other order which is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. To the extent such publications may set out definitions of a term that conflict with the explicit or implicit definition of the present disclosure, the definition of the present disclosure controls. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### DEFINITIONS

The term “distal” as used herein refers to the end of a device (e.g., a sinus dilator device or insertion device), or a component thereof, that is positioned towards the end of the device that is inserted through or closest to a sinus opening of the subject and which is positioned furthest from the medical technician (e.g., a surgeon) who is placing the dilator. For example, the distal end of a sinus dilator is the end of the device that is first inserted into the frontal sinus recess of the subject.

The terms “sinus opening” and “sinus ostium” are used interchangeably herein to refer to the opening and/or passageway from the nasal cavity to a sinus cavity, such as a frontal sinus cavity, a maxillary sinus cavity or a sphenoid sinus cavity.

The terms “frontal sinus recess” as used herein refers to the passageway from the nasal cavity to the frontal sinus cavity, including the innermost opening into the frontal sinus cavity.

The terms “insert” or “insertion” are used herein interchangeably to describe the positioning of a dilator in a stenotic sinus opening or stenotic frontal sinus recess of a subject.

The terms “osmotic agent,” “osmotically active agent” and “osmoagent” are used interchangeably herein and refer to an agent that facilitates the imbibition of water from a region of high water potential (e.g., low solute concentration) through a semipermeable membrane to a region of low water potential (e.g., high solute concentration) until a state of dynamic equilibrium is reached. In some instances, the osmotically active agent may be configured to absorb water flowing through a semipermeable membrane from the surrounding tissues after insertion of a dilator into a stenotic frontal sinus opening or recess of the subject and expand.

The term “proximal” as used herein refers to the end of the dilator device, or a component thereof, which is positioned towards the end of the dilator that remains on the nasal cavity side of the stenotic opening or is closest to the medical technician (e.g., a surgeon) who is placing the dilator. For example, the proximal end of a sinus dilator is the end that remains on the nasal cavity side of the stenotic frontal sinus recess when the sinus dilator device is positioned in the stenotic recess during use.

The term “self-expanding”, when referring to a dilator or a driving mechanism for dilating the dilator means that the dilator expands from a non-expanded configuration to an expanded configuration without external intervention from a user or a medical technician (e.g., a surgeon). For example, the self-expanding driver may be self-contained, such that the driver is configured to expand without connection to an external pressure source. As such, self-expanding drivers as described herein function without the need for an external pressure source or a pressure monitoring device (e.g., as with a balloon catheter). In some cases, the self-expanding driver expands from the non-expanded configuration to the expanded configuration upon absorbing fluid from the surrounding environment when the device is in use. For instance, the self-expanding driver may expand from the non-expanded configuration to the expanded configuration upon absorbing water from the surrounding tissues of the stenotic opening when the device is in use. Self-expanding drivers may be configured to expand the expandable portion of the device by various ways, such as by osmosis and the use of an osmotic agent.

The terms “stenotic opening” and “stenotic recess” refer to an abnormal narrowing of a sinus opening or frontal sinus recess.

### DETAILED DESCRIPTION

Devices that are adapted to insert a sinus dilator into a stenotic opening and/or recess of a frontal sinus in a subject patient using minimally invasive insertion procedures are provided. The devices and methods can be used to treat sinusitis and other nasal and/or sinus disorders.

When inserting a frontal sinus recess dilator, the frontal recess, a passageway leading from the nasal cavity to the frontal sinus cavity, may be variable from person to person, and is typically non-linear. In addition, the tissue surrounding the passageway leading from the nasal cavity to the frontal sinus cavity tends to be bony with a thin mucosal layer covering the bone. As a result, insertion of a straight rigid (i.e., relatively non-flexible and non-bendable) dilator tends to scrape and bruise or potentially break the bony tissue surrounding the non-linear passageway leading up to the frontal sinus opening. A certain degree of flexibility and bendability in a frontal sinus recess dilator is desirable to minimize tissue damage during dilator placement. On the other hand, too much flexibility and bendability in a frontal sinus recess dilator may cause more difficulty in advancing the dilator through the passageway extending from the nasal cavity to the frontal sinus cavity. This difficulty is similar to that encountered when inserting a flexible string through a long thin, non-linear hole or passageway.

Osmotically-driven sinus dilators that have a central tube made from metal or rigid plastic so as to withstand the pressures generated by the surrounding osmotic driver may result in the dilator being relatively non-flexible and non-bendable. Although such non-flexible and non-bendable dilators are useful for the treatment of the maxillary sinus ostium, which typically has a length of about 5 mm, such dilators may be more difficult to place into a frontal sinus recess, an anatomical passageway that is typically tortuous and has a longer length, e.g., 15 to 17 mm.

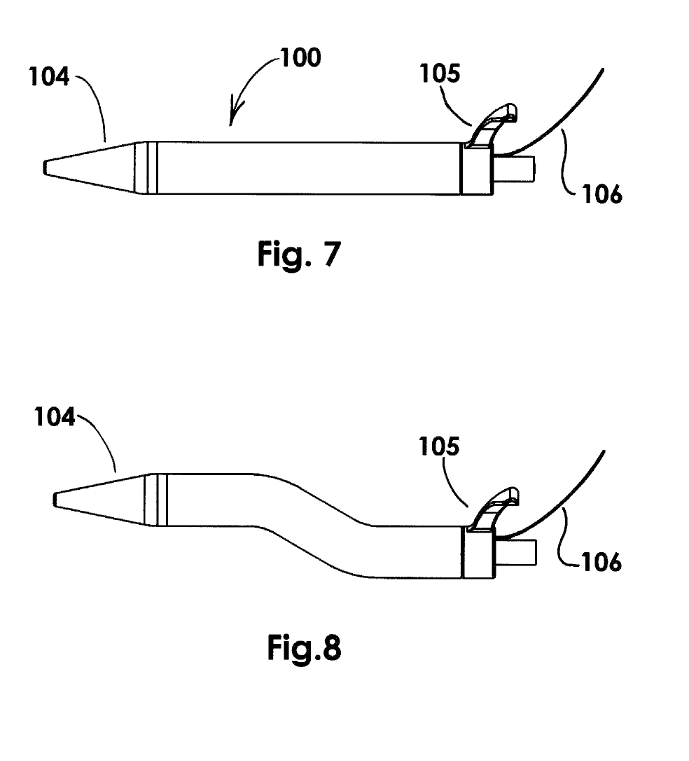

The dilator 100 (see FIG. 7) described herein may have some of the same features as previously described osmotically-driven sinus ostium dilators, for example a tapered distal tip, an osmotic driver comprised of an inner membrane, an outer elastic semipermeable membrane, and osmotic tablets, which may perform similar functions as their counterparts in previously described dilators. See, e.g., U.S. Application Publication No. 2013/0231693 and U.S. Application Publication No. 2012/0053567, the disclosures of each of which are incorporated herein by reference. Dilators of the present disclosure have an increased flexibility and bendability, which can be imparted by the use of a malleable axial member 101 in place of rigid tubes that were used in previous osmotic dilators (see FIG. 10). In some instances, axial member 101 resists the radial compression forces encountered during the expansion of the tablets 112 so that the interior lumen of the member 101 remains open during expansion of dilator 100.

While the axial member 101 may have the desired malleability as outlined above, the annular osmotic tablets 112 positioned around the axial member 101 may be configured so as to allow the dilator 100 to bend. In this regard, in some embodiments, annular-shaped compressible spacers 114 are positioned between the osmotic tablets 112. The spacers 114 can be made from a compressible material, such as a polymer and/or a rubber that may be easily compressed and/or flexed. In those cases, where the dilator 100 is to be inserted for relatively short periods of time (e.g., 4 hours or less), the spacers 114 may be made from elastomeric materials that form strong bonds to inner membrane 111 and outer membrane 113. Suitable materials for spacers 114 include, but are not limited to, polyesters, polyurethanes, polyethers, closed cell polyester polyurethane and polyether foams, and the like.

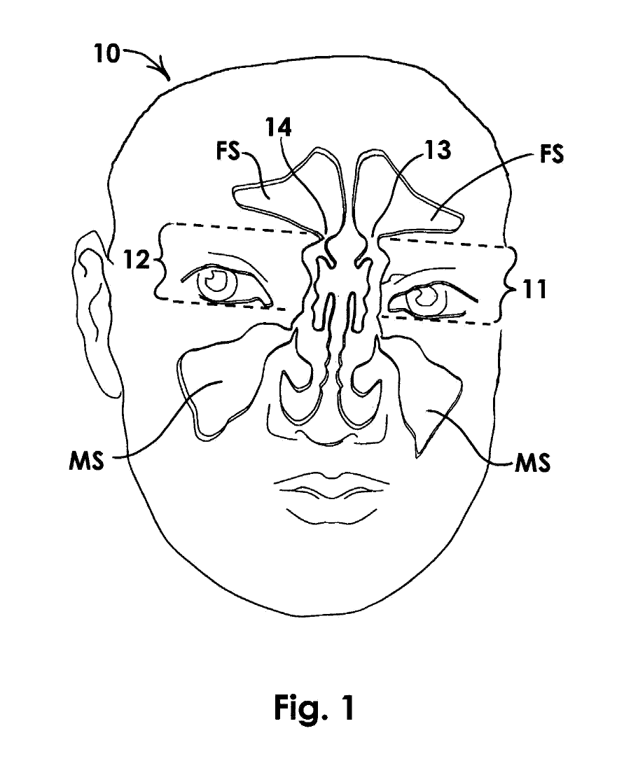

Referring now to FIG. 1, there is shown a human patient 10 having two frontal sinuses (FS) and two maxillary sinuses (MS). Each of the frontal sinuses have a recess which can be accessed by way of the patient's nostrils; frontal recess 11 has at the entrance to the left frontal sinus cavity an opening 13, whereas frontal sinus recess 12 has at the entrance to the right frontal sinus cavity an opening 14. Frontal sinus recess 11 is shown in a normal open condition (see, e.g., opening 13) and frontal sinus recess 12 is shown in an occluded or stenotic condition (see, e.g., opening 14).

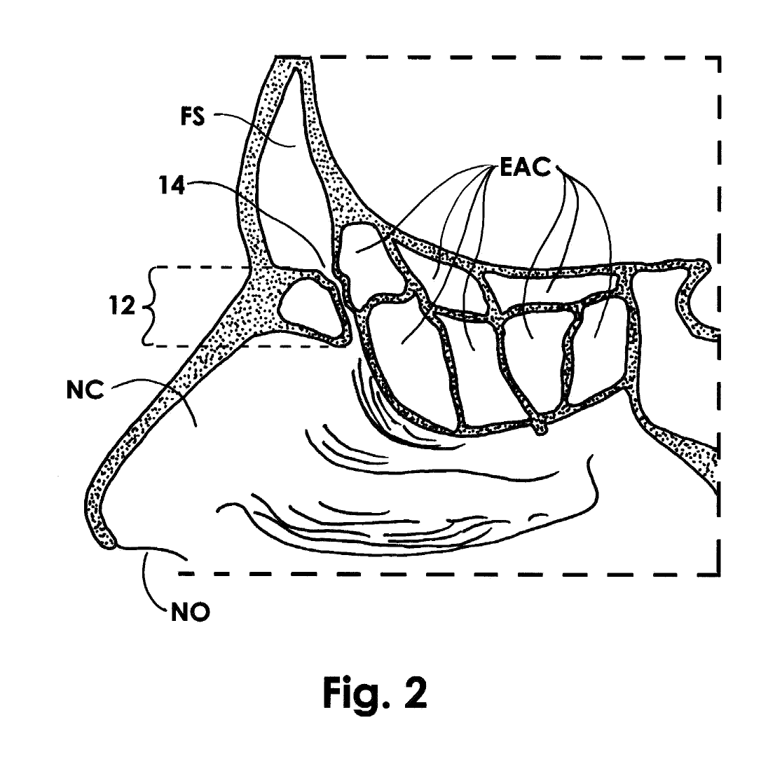

Referring now to FIG. 2, there is shown a sectional view of a patient's nose and sinuses including the nasal cavity (NC), the nostril opening (NO), the right frontal sinus (FS) and the ethmoid air cells (EAC). As was shown in the frontal view of FIG. 1, the right frontal sinus recess 12 is shown in a partially occluded or stenotic condition, which may inhibit proper drainage from the frontal sinus cavity (FS).

### Devices and Methods for Inserting a Sinus Dilator

Aspects of the present disclosure include an insertion device adapted to insert a sinus dilator into a stenotic opening of a paranasal sinus in a subject patient using minimally invasive insertion procedures. For example, the insertion device can be used to insert a sinus dilator into a stenotic frontal sinus recess. The insertion device and methods can be used to treat sinusitis and other nasal and/or sinus disorders.

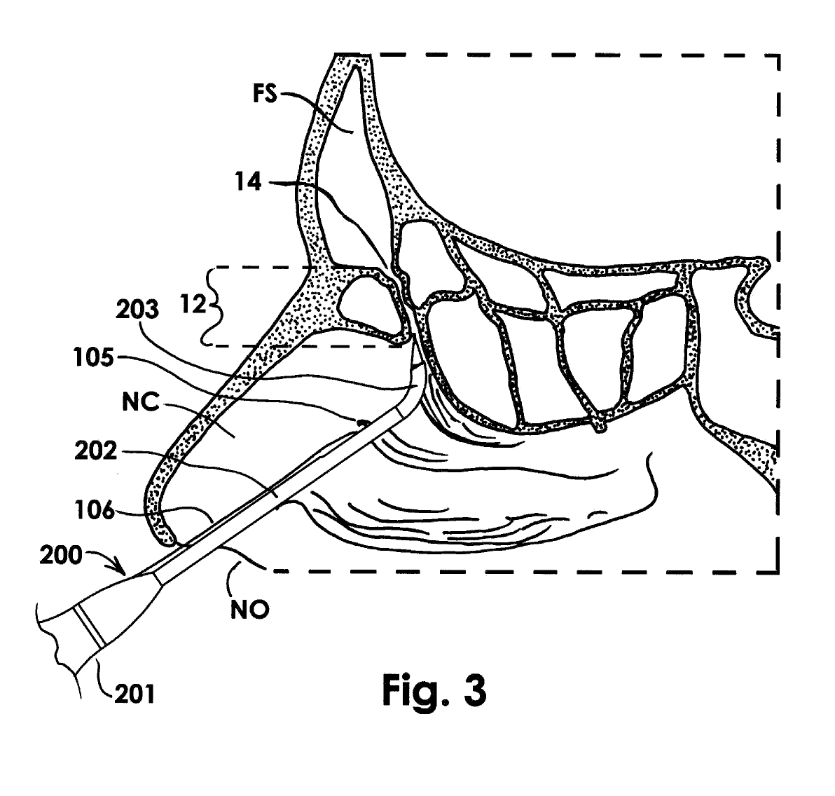

Referring now to FIG. 3, there is shown the same sectional view of the patient's nose and sinuses as was shown in FIG. 2, but in addition, there is also shown a dilator insertion device 200 having a handle 201 and a tube 202 having an angled distal end 203. In certain embodiments, the proximal end of the tube 202 may be coupled to handle 201 and the distal end of the tube 202 is dimensioned to pass through a nasal cavity (NC) of the patient. Positioned within tube 202 is a dilator 100 (shown in FIG. 7 but substantially hidden from view in FIG. 3) having a tapered distal tip 104, a proximal anchor 105 and a tether 106. The distal end 203 of the insertion device is curved to an angle of approximately 70° in relation to the axis of tube 202 which facilitates placement of the dilator 100 in the frontal sinus recess 12. The tube 202 extends through the nostril opening (NO) to a handle 201 which is grasped and manipulated by the healthcare provider (e.g., surgeon).

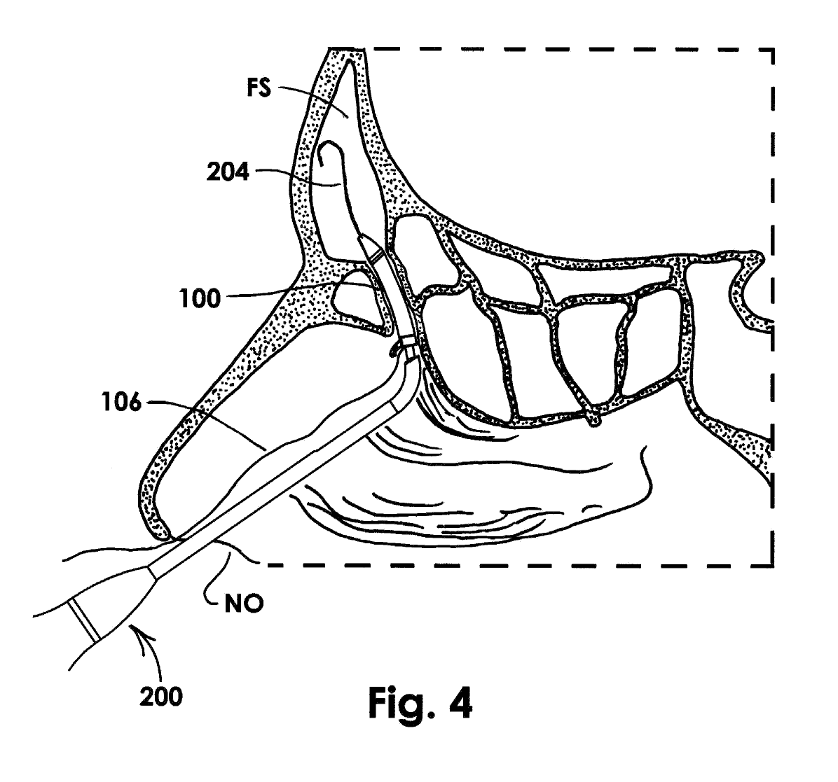

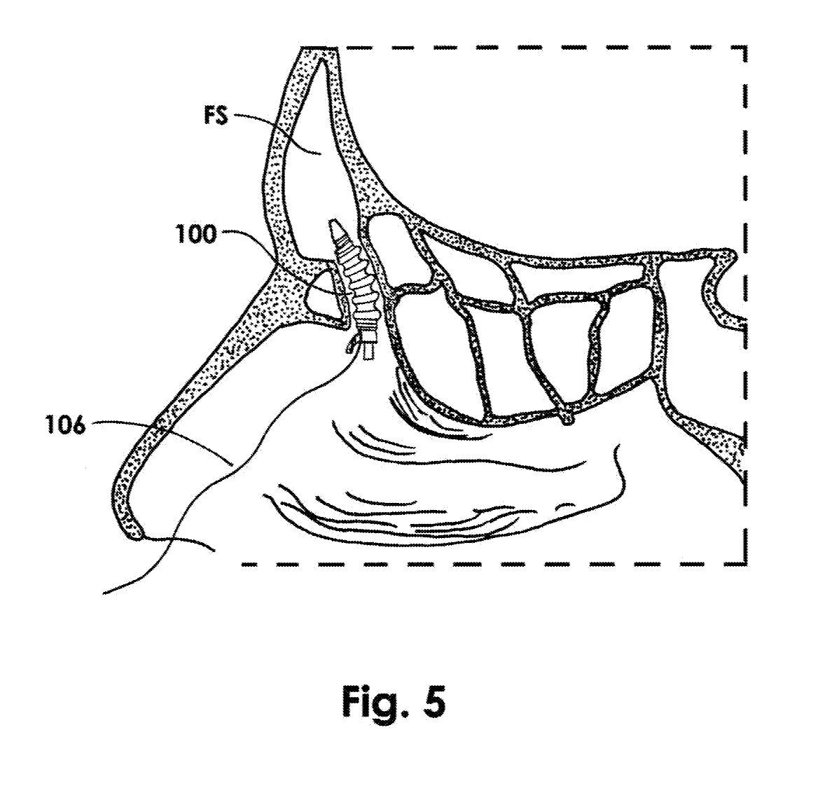

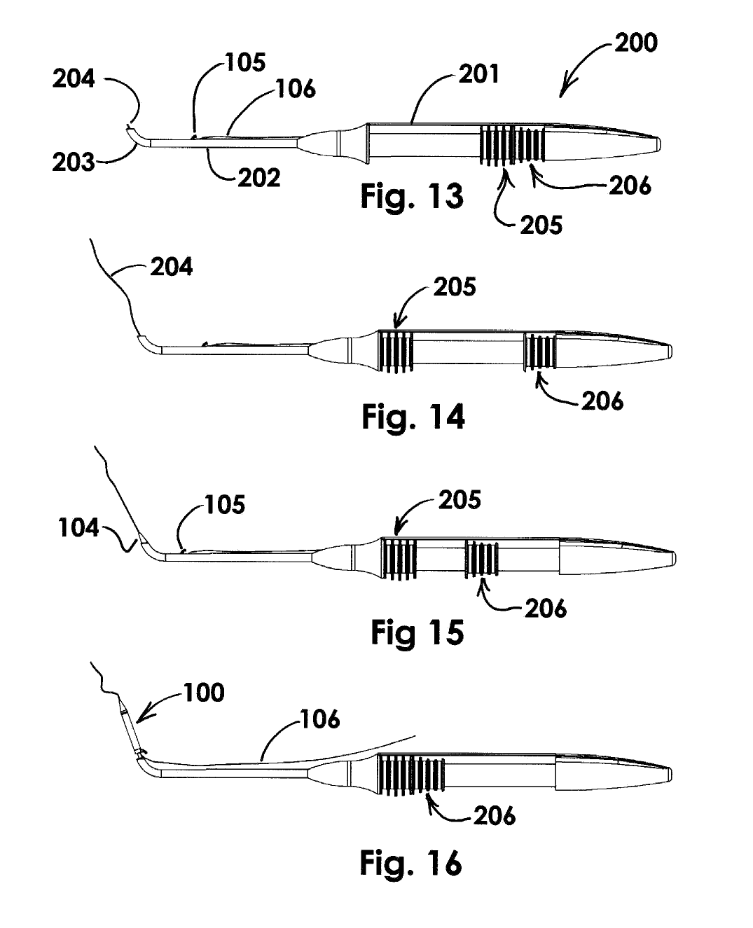

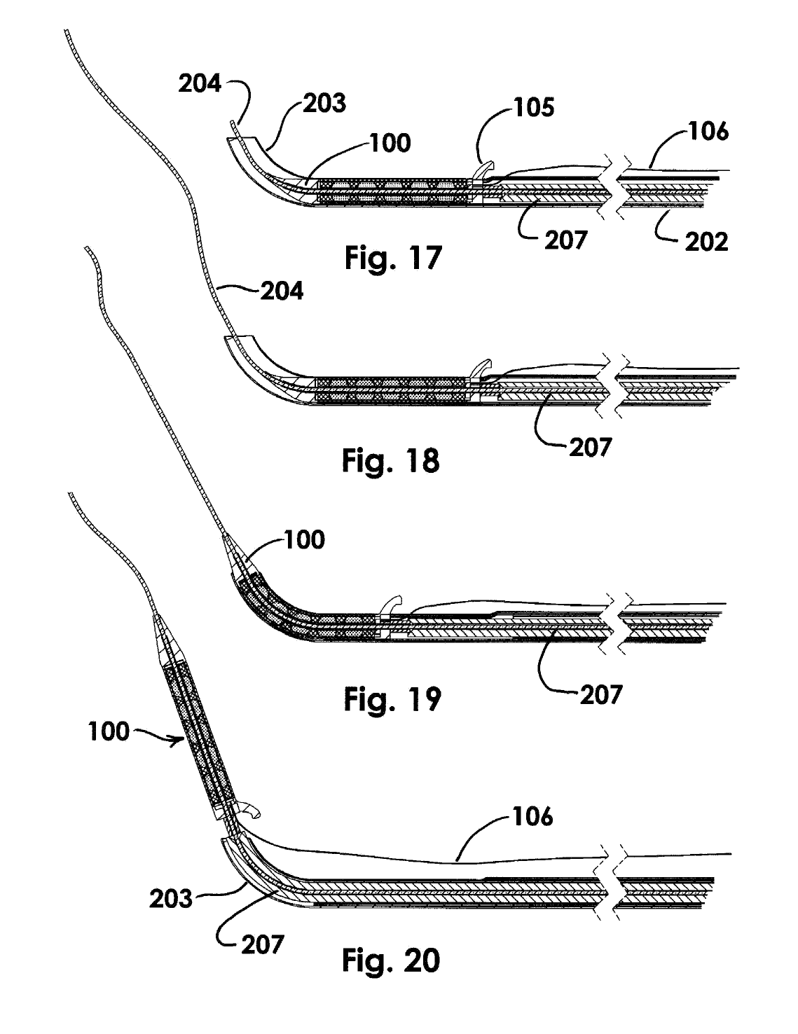

Insertion of a frontal sinus dilator 100 into a stenotic frontal sinus recess will now be described while referring to FIGS. 3-5 and FIGS. 13-20. The insertion device 200 shown in FIG. 3 is shown in greater detail in FIGS. 13 and 17. The insertion device 200 includes a handle 201 a tube 202 having an angled distal end 203. Extending out of opening in distal end 203 is the distal end of guidewire 204. Mounted on the handle 201 are a guidewire advancing actuator 205 and a dilator deployment actuator 206. As can be seen in FIGS. 3, 13 and 17, the dilator 100 is positioned substantially within the tube 202 with only the proximal anchor 105 and tether 106 extending outside of the tube. The anchor 105 extends through a slot in tube 202 and distal end 203. Once the healthcare provider (e.g., surgeon) has the insertion device 200 positioned in the patient's nasal cavity such that the tip of angled distal end 203 is near the start of the frontal sinus recess 12, the healthcare provider (e.g., surgeon) may then slide actuator 205 in a distal direction, which causes the guidewire 204 to advance in a distal direction, where it extends beyond the opening in angled distal end 203, as shown in FIGS. 14 and 18. In the context of the nasal cavity, extending the guidewire 204 from the position shown in FIG. 3 causes the guidewire 204 to extend through the frontal sinus recess 12 and into the frontal sinus cavity (FS). Once the guidewire 204 is extended through the frontal sinus recess 12, the healthcare provider (e.g., surgeon) may slide actuator 206 in a distal direction, which causes arm 207 to move distally and push the dilator 100 in a distal direction. In some cases, an electrical circuit can be created to actuate the mechanical translation of the arm 207, such as, for example by electrical actuation of a solenoid element. Arm 207 may be flexible so that it can bend while advancing through the angled distal end 203 as shown in FIG. 20. The arm also has an axial bore which accommodates the guidewire 204 extending therethrough. The distally advancing arm 207 pushes the dilator 100 in a distal direction, causing the dilator 100 to slide over the extended guidewire 204, as shown sequentially in FIGS. 19 and 20. In the context of the nasal cavity, sliding the actuator 206 in a distal direction causes the dilator 100 to slide over the extended guidewire 204 and into the frontal sinus recess 12, as shown in FIG. 4. Once the dilator is in place within the frontal sinus recess 12, the healthcare provider (e.g., surgeon) may pull the insertion device 200 out of the patient's nasal cavity, leaving the dilator 100 in place within the frontal sinus recess 12. Optionally, the guidewire 204 can be retracted back into the tube 202 and distal end 203 by sliding the actuator 205 in a proximal direction before the insertion device 200 is pulled out of the nasal cavity.

As summarized above, the insertion device 200 is dimensioned such that at least the distal end of the device can pass through the nasal cavity (NC) of a subject. The distal end may include, for example, at least a portion of the tube 202 and arm 207. As such, at least the distal end of the device has a cross-sectional diameter that is 10 mm or less, such as 8 mm or less, and including 5 mm or less. The arm 207 may have the same outer cross-sectional dimensions (e.g., diameter) along its entire length. Alternatively, the cross-sectional diameter may vary along the length of the arm 207.

Furthermore, the lengths of the tube 202 and the arm 207 may vary. For example, the lengths of the arm may vary depending on the specific sinus being targeted (e.g., frontal sinus). In some instances, the lengths of the arm ranges from 1 cm to 20 cm, such as 2 cm to 15 cm, including 5 cm to 10 cm. It should be appreciated that in some instances the tube 202 and arm 207 may have different lengths from one another. In embodiments for accessing the frontal sinus recess, the tube 202 including the angled distal end 203 may have a length in the range of 7 cm to 11 cm.

As stated above, the tube 202 and arm 207 of the insertion device 200 each have a proximal end and a distal end. The terms “proximal” and “proximal end”, as used herein, refers to the end of the insertion device or other component on the insertion device that is nearer the user (such as a physician operating the device in an insertion procedure), and the terms “distal” and “distal end”, as used herein, refer to the end of the insertion device or other component on the insertion device that is nearer the target stenotic sinus opening and/or frontal recess of the subject during use.

The tube 202 may be, for example, a structure of sufficient rigidity to allow the distal end to be pushed through tissue when sufficient force is applied to the proximal end of the insertion device. As such, in some embodiments, the tube 202 is not significantly pliant or flexible. Example materials may include, but are not limited to, metals, metal alloys (e.g., stainless steel), polymers such as hard plastics, etc.

In some embodiments, the tube 202 includes a curved section at its distal end 203. The curvature and length of curvature may vary in degree, and may vary according to application, such as with which sinus opening that is being accessed, e.g., maxillary sinus, frontal sinus, sphenoid sinus, etc. In some embodiments, to facilitate access to the frontal sinus recess, the distal end 203 of tube 202 is configured to bend at an angle ranging from 0° to 140°, such as 10° to 130°, including 20° to 120°, or 30° to 110°, or 40° to 100°, or 50° to 90°, or 60° to 80°, such as 70°, from the axis (e.g., longitudinal axis) of the non-curved portion of tube 202. In other embodiments, to facilitate access to an opening of the maxillary sinus, the distal end 203 of tube 202 is configured to bend at an angle ranging from 0° to 150°, such as 10° to 130°, including 20° to 120°, or 30° to 120°, or 60° to 120°, or 90° to 120°, or 100° to 120°, or 105° to 115° from the axis of the non-curved portion of tube 202. In some embodiments, the distal end 203 of tube 202 is configured to bend at an angle ranging from 105° to 115°, such as 110°, from the axis of the non-curved portion of tube 202. In some cases, the length of the distal end 203 (e.g., the arc length of the curved distal end 203) is 5 cm or less, such as 3 cm or less, including 2 cm or less, or 1 cm or less, or 0.5 cm or less. As such, in the above embodiments, when the sinus dilator is coupled to the insertion device, the sinus dilator may be positioned at an angle relative to the axis of tube 202. The angle may be in the ranges and values described above. In some embodiments, the curved distal end 203 has little to no flexibility, i.e., the curved distal end 203 is rigid to semi-rigid allowing minimal flex to provide support and guidance for the dilator 100 as the dilator is advanced out of the distal end 203.

The arm 207 may be, in some instances, a structure of sufficient rigidity to allow the sinus dilator to be pushed through the stenotic opening when sufficient force is applied to the proximal end of the device, even when the stenotic opening is completely occluded. In some instances, the arm 207 may be a metal, metal alloy, polymer (hard or pliant and flexible), etc. Further, the arm 207 is, in some instances, a structure sufficiently pliant and flexible such that the arm 207 may be relatively displaced in tube 202 having a curved or angled distal end 203. Examples of sufficiently pliant and flexible materials may include, but are not limited to, polymers such as plastics, rubber or rubber-like polymers, flexible metal (e.g., flexible wire), etc. In such cases, the arm 207 may provide the rigidity necessary to push the sinus dilator through the stenotic opening with sufficient force applied to the proximal end of the insertion device.

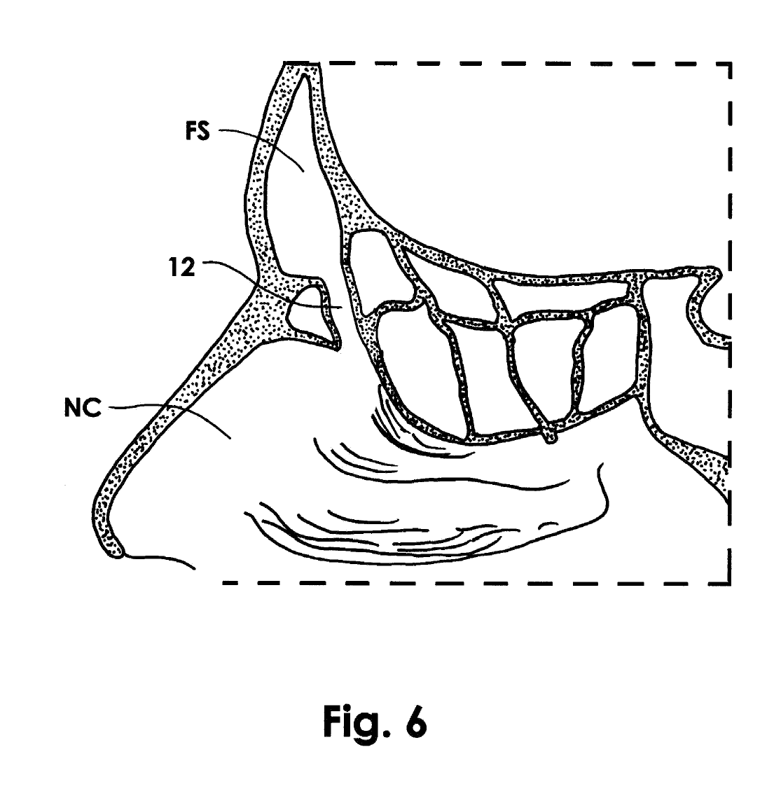

In certain embodiments, during operation, the dilator 100 expands by way of an osmotic driver 110. The expansion of dilator 100 is gradual and takes place over a period of time, typically 0.5 hours or more, and in some instances 1 hour or more. Once fully expanded, the dilator may be pulled out of the frontal sinus recess 12 by pulling on the tether 106 which can be left extending out of the patient's nostril opening (NO) while the dilator 100 is in use. After dilator 100 removal, the frontal sinus recess 12 is opened to a non-stenotic condition allowing natural ventilation and drainage of the frontal sinus (FS) into the nasal cavity (NC), as shown in FIG. 6.

The operation of the dilator 100 is now described in greater detail referencing FIGS. 7 through 12. FIGS. 7 to 10 show the dilator 100 in a non-expanded configuration which is the configuration at the time the dilator 100 is first positioned within a frontal sinus recess 12. Dilator 100 has a tapered distal tip 104 to facilitate insertion into a stenotic frontal sinus recess 12. The proximal anchor 105 is configured to prevent the dilator 100 from being inserted too far into the frontal sinus recess 12. The tether 106 may be used to ensure that the dilator 100 is not inhaled or swallowed, or otherwise lost, should the dilator 100 become dislodged from frontal sinus recess 12. The tether 106 may also be used to pull the dilator 100 out of the frontal sinus recess 12 after the frontal sinus recess 12 has been dilated. Alternatively, the healthcare provider (e.g., surgeon) can grab the proximal end of the dilator 100 with forceps in order to remove the dilator 100 from frontal sinus recess 12. Sleeve 108 is composed of a rigid material (e.g., rigid plastic) and slides over axial member 101 and abuts against anchor 105. Sleeve 108 is affixed to axial member 101 either by chemical bonding or mechanical flaring of the proximal end of axial member 101. Sleeve 108 prevents axial expansion of the osmotic driver 110 in a proximal direction during dilation of dilator 100, for example in embodiments where anchor 105 is not fixedly attached to axial member 101.

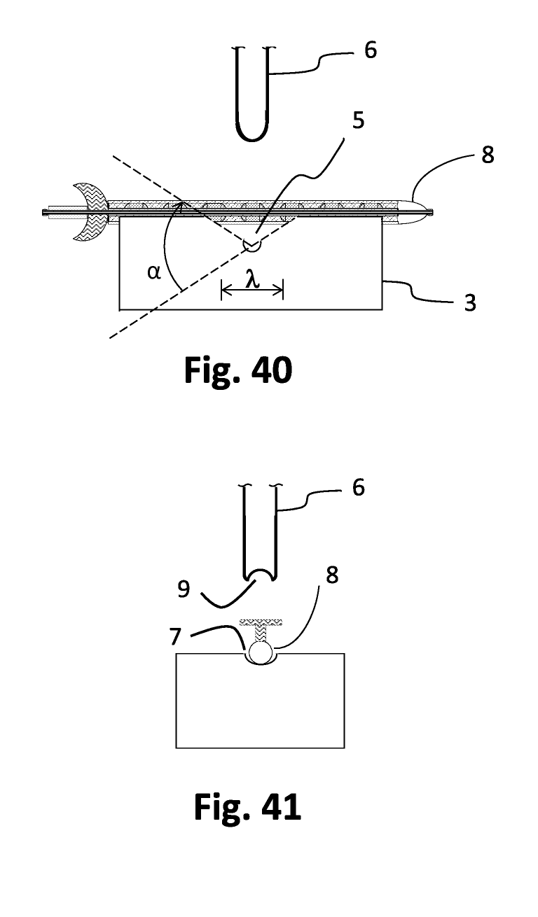

The dilator 100 can be bent at multiple points along its length and therefore can be bent into various orientations. For example, in the embodiment shown in FIG. 8, the dilator 100 has two bends along its length. In some instances, the maximum bend angles are about 30°. The dilator can be bent at any particular flex/bend point at an angle that is less than or equal to the maximum bend angle. The bendability of dilator 100 facilitates placement of the dilator into the frontal sinus recess 12, as the frontal recess in most patients is a non-linear passageway, lined with boney tissue, and having bends therein. By being bendable, the dilator 100 can assume the non-linear configuration of the patient's stenotic frontal recess which helps to minimize tissue damage upon insertion of dilator 100 into frontal sinus recess 12. Additionally, dilator bendability allows the dilator 100 to conform to the natural anatomical configuration of a frontal recess in a particular patient while dilating the stenotic frontal sinus recess 12. The bendability of a dilator can be expressed in terms of its malleability. In certain embodiments, the dilator 100 has a malleability of 30 Newtons (N) or less as measured using the measurement method described herein and the malleability measurement apparatus shown in FIGS. 40 and 41. For example, the dilator may have a malleability of 30 N or less, or 27 N or less, such as 25 N or less, or 23 N or less, or 20 N or less, or 17 N or less, or 15 N or less, or 13 N or less, or 10 N or less, or 7 N or less, or 5 N or less. In other embodiments, the dilator 100 has a malleability of 5 Newtons or more. For instance, the dilator may have a malleability of 7 N or more, or 10 N or more, or 13 N or more, or 15 N or more, or 17 N or more, or 20 N or more, or 23 N or more, or 25 N or more, or 27 N or more. In some embodiments, the dilator 100 has a malleability of 5 N or more. In some embodiments, the dilator 100 has a malleability of 10 N or more. In some embodiments, the dilator 100 has a malleability of 15 N or more. In some embodiments, the dilator 100 has a malleability of 20 N or more. In some embodiments, the dilator 100 has a malleability of 15 N to 30 N, such as 17 N to 27 N, including 20 N to 25 N. In some instances, the dilator has a malleability of 20 N to 25 N. In some instances, the dilator has a malleability of 23 N. In some embodiments, the dilator 100 has a malleability of 5 N to 30 N, such as 5 N to 25 N, including 10 N to 20 N, or 10 N to 15 N. In some instances, the dilator has a malleability of 10 N to 20 N. In some instances, the dilator has a malleability of 10 N to 15 N. In some instances, the dilator has a malleability of 11 N.

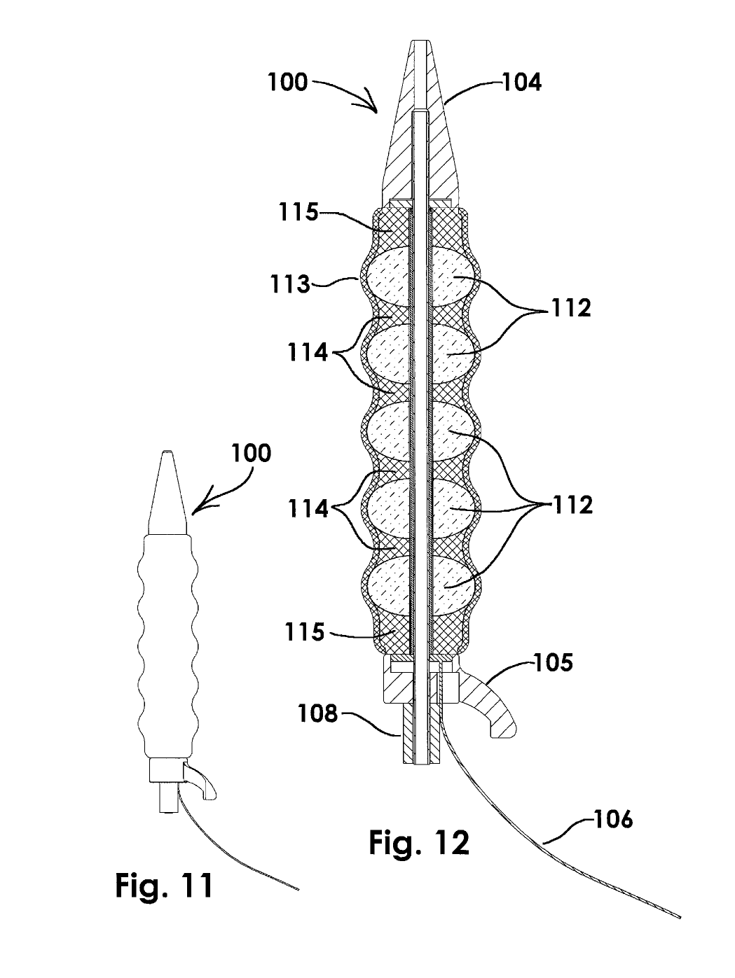

Although the dilator 100 shown in FIGS. 7 to 10 is substantially cylindrically-shaped, the dilator can also have a non-cylindrical shape, for example a shape having a roughly circular cross-section, but where the diameter of the cross-section varies along the length of the dilator. One such circular-cross section shape is a frustum shape, but other shapes such as the shape shown in FIGS. 11 and 12 can also be used.

As shown in FIG. 10, dilator 100 includes a malleable axial member 101. Although the axial member 101 shown in FIG. 10 is in the form of a tube which can accommodate a guidewire extending therethrough, the axial member 101 can also be a solid malleable rod, or a malleable coil.

One example of a malleable axial member 101 suitable for use in an osmotically-driven frontal sinus recess dilator as described herein is a stainless steel tube. In some instances, the tube has an inside diameter of 0.5 mm (0.020 in) and an outside diameter of 0.6 mm (0.025 in). Malleability may be imparted to the stainless steel tube by annealing the tube at temperatures ranging from 950 to 1050° C. (1750 to 1925° F.).

The term “malleable” or “malleability” as used herein means that a material or device (e.g., a dilator or a component thereof) is flexible, and once flexed, it bends and substantially retains its bent shape. Malleability is measured using a load cell which imparts a force perpendicular to the axis of a test piece (e.g., a dilator 100 or a component thereof such as the axial member 101) while the test piece is held in a holding jig that permits the test piece to be bent at an angle of 30°, measured from the axis of the test piece. Where the test piece includes an osmotic dilator and a semipermeable outer membrane, testing is conducted under ambient conditions of temperature and at 40% to 80% relative humidity. The measurement method and apparatus are as follows.

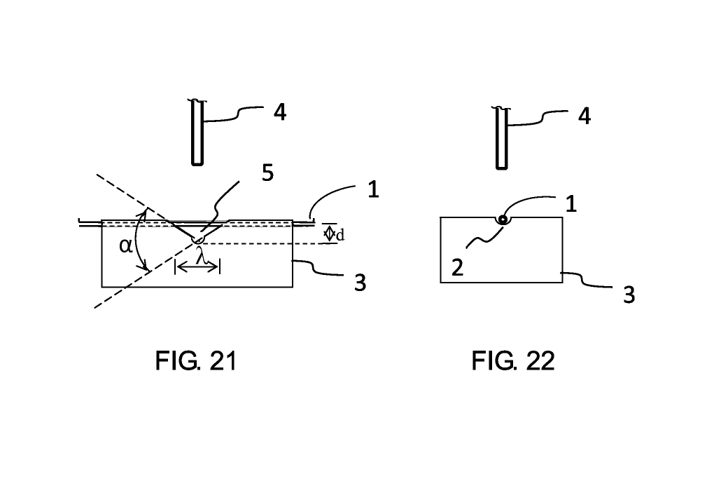

The measurement method for a sample stainless steel tube is described as follows. Referring to FIGS. 21 and 22, a sample stainless steel annealed tube 1 is placed on a holding jig 3 having a V-shaped depression 5 in its upper surface. The tube 1 sits in a shallow groove 2 which keeps the tube 1 secure during bending. When positioned within groove 2, the tube 1 spans across depression 5. The depression 5 has a length λ of 10.5 mm and a depth d of 1.4 mm. The surfaces of the depression are each cut at an angle of 15° with respect to the top surface of jig 3, so that the maximum bending angle to which tube 1 is subjected is angle α, i.e., 30°. A vertically moveable probe 4 having a round cross-section and a diameter of 2.6 mm (101 mils) is connected to a load cell (Chatillon TDC119 Force Measurement System; maximum load of 100 Newtons). Once the tube 1 is nested in groove 2, the probe 4 is centered over the tube and the center of the depression 5, as shown in FIG. 21. When measuring malleability of a tube 1 itself, the tip of the probe 4 may be substantially flat. In other instances, when measuring the malleability of a dilator (see FIGS. 40 and 41), the tip of the probe 6 may be curved (e.g., concave) to conform to the curvature of the outer surface of the dilator 8. Referring back to FIGS. 21 and 22, the probe 4 is then lowered to “just touch” the tube 1 and the instrument's displacement setting is set to zero. Then the probe 4 is lowered gradually and displacement versus load is recorded within approximately 1-2 seconds. The tube 1 is visually monitored until the sides of the tube are in contact with the sloping walls of the V-shaped depression 5. Displacement at this 30° bend is recorded. The measured load at this point is recorded as the end point of the test. Displacement is increased beyond this point until the tube is bent more than 30° to complete the malleability profile. For example, a stainless steel tube annealed at 955° C. (1750° F.) was characterized according to the above procedures. After plotting the load versus displacement data, the displacement to reach a 30° bend was 1.0 mm. This corresponded to a load of 3.8 Newtons, which represents the malleability value of the tube.

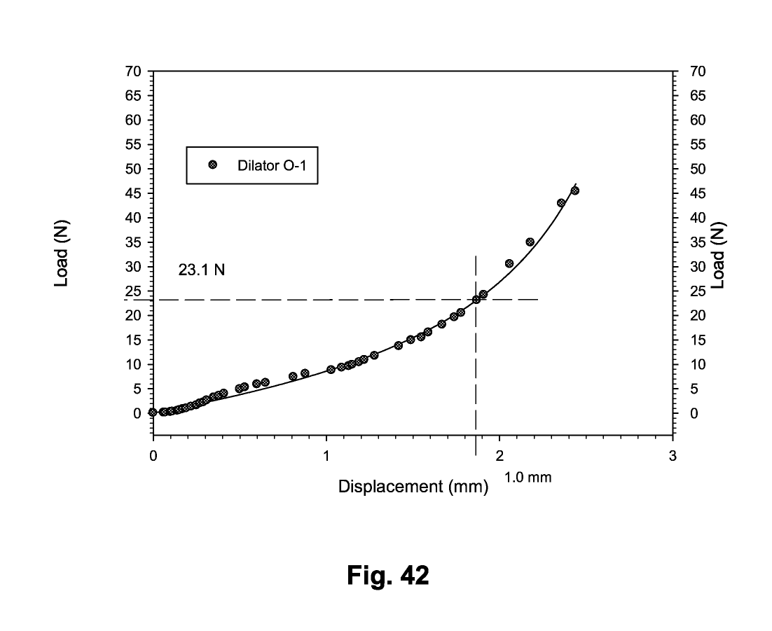

The malleability of a dilator according to embodiments of the present disclosure is measured as follows. Referring to FIGS. 40 and 41, a dilator 8 is placed on a holding jig 3 having a V-shaped depression 5 in its upper surface. The dilator 8 sits in a shallow groove 7 which keeps the dilator 8 secure during bending. When positioned within groove 7, the dilator 8 spans across the V-shaped depression 5. The depression 5 has a length λ of 10.5 mm. The surfaces of the depression 5 are each cut at an angle of 15° with respect to the top surface of jig 3, so that the maximum bending angle to which dilator 8 is subjected is angle α, i.e., 30°. A vertically moveable probe 6 having a round cross-section and a diameter of 4.93 mm is connected to a load cell (Chatillon TDC119 Force Measurement System; maximum load of 100 Newtons). Once the dilator 8 is nested in groove 7, the probe 6 is centered over the dilator 8 and the center of the depression 5, as shown in FIG. 40. When measuring the malleability of a dilator, the tip 9 of the probe 6 has a shape (e.g., curved, such as concave) to conform to the curvature of the outer surface of the dilator 8. The probe 6 is then lowered to “just touch” the dilator 8 and the instrument's displacement setting is set to zero. Then the probe 6 is lowered gradually and displacement versus load is recorded within approximately 1-2 seconds. The dilator 8 is visually monitored until the dilator is bent to 30°. Displacement at this 30° bend is recorded. The measured load at this point is recorded as the end point of the test. Displacement is increased beyond this point until the tube is bent more than 30° to complete the malleability profile. For example, a dilator made with an axial member (i.e., a 304 stainless steel tube with an outside diameter of 0.6 mm and an inside diameter of 0.5 mm) annealed at 955° C. (1750° F.) was characterized according to the above procedures. After plotting the load versus displacement data, the displacement to reach a 30° bend was 1.87 mm. This corresponded to a load of 23.1 Newtons, which represents the malleability value of the dilator (see FIG. 42).

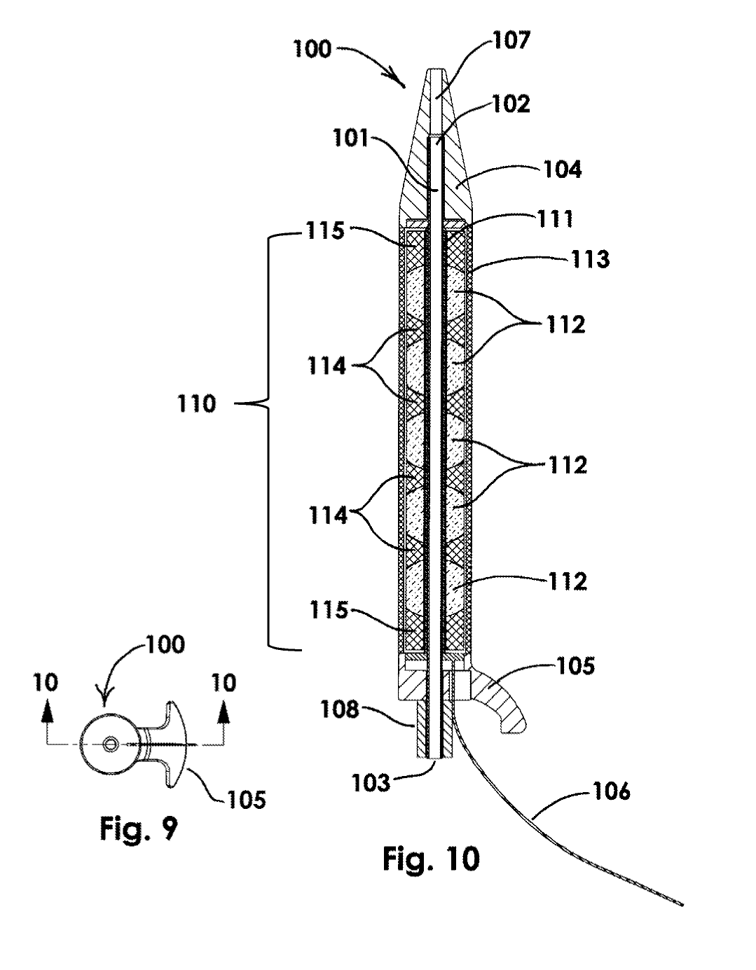

In the case of the hollow tube embodiment shown in FIGS. 9 and 10, the axial member 101 has a distal opening 102 and a proximal opening 103. As is explained in more detail herein, axial member 101 has an inner diameter sufficiently large to permit a guidewire 204 to pass through the dilator 100 while the dilator is being positioned into the frontal sinus recess 12. For example, guidewires typically have diameters in the range of 0.4 to 0.8 mm so to accommodate guidewires of this size range, the axial member 101 may have an inner diameter of 0.5 to 0.9 mm, or even larger. The tubular axial member 101 is substantially non-collapsible under the pressures exerted by the osmotic driver 110 during use, so that as osmotic pressure is generated within driver 110, it causes the dilator 100 to expand radially outwardly from the axial member 101 rather than causing the axial member 101 to collapse or significantly decrease in diameter.

Referring back to FIG. 10, surrounding the distal end of axial member 101 is a tapered distal tip 104 having a bore 107 that is in communication with hollow interior portion of axial member 101. Like the inner diameter of axial member 101, the bore 107 also has a sufficient diameter to accommodate a guidewire extending therethrough. At the proximal end of dilator 100 is a proximal anchor 105 having a radially extending member, which is shown in FIGS. 9 and 10. Alternatively, the dilator can also have a pair of radially extending members which extend in opposite directions from the central axis of dilator 100 (as shown in dilator 8 in FIG. 40). Proximal anchor 105 has a sufficient length compared to the unexpanded diameter of the osmotic driver and thereby prevents the dilator 100 from being inserted too far into the frontal sinus recess 12, thereby preventing the distal tip 104 from impacting and/or damaging the frontal sinus cavity. The hollow interior of axial member 101 also creates a conduit or passageway for fluid and/or gas, such as air, mucus, puss and/or blood, to pass through the dilator 100 after the dilator 100 is placed within the frontal sinus recess 12 and the insertion device 200 is removed.

Positioned along a central portion of axial member 101 (e.g., between the distal tip 104 and the proximal anchor 105 is an osmotic driver 110 that includes an inner membrane 111 and an outer elastic semipermeable membrane 113 that together surround an osmotic core comprised of a plurality of osmotic tablets 112. The osmotic tablets 112 may include one or more osmotically active agents such as water soluble salts or sugars, such as sodium chloride, sorbitol, etc., and optionally binders, lubricants and mold release agents. The osmotic tablets 112 additionally may include osmopolymers such as, but not limited to, polyethylene oxide, sodium carboxymethyl cellulose, hydroxypropyl cellulose (e.g., low substituted hydroxypropyl cellulose), combinations thereof, and the like. Certain embodiments of the osmotic tablets 112 include annularly (e.g., donut) shaped salt- and/or polymer-containing tablets having an inner opening that is large enough to slide over axial member 101 coated with inner membrane 111. In some instances, the tablets 112 have an outer diameter of 5 mm or less, such as 4 mm or less, or 3 mm or less, or 2 mm or less. For instance, the tablets 112 may have an outer diameter of 3 mm. In some instances, the tablets are composed of an osmotically active salt (e.g., NaCl) and/or polymer, such as a high molecular weight hydrogel-forming polymer, for example polyethylene oxide (e.g., Polyox™, Dow Chemical Company, Midland, Mich.). In certain cases, the osmotic tablets 112 include tableting excipients and/or lubricants. In some embodiments, the osmotic tablets 112 include 10 to 95 wt % salt, such as 20 to 90 wt % salt, including 30 to 80 wt % salt, or 40 to 70 wt % salt. For example, the osmotic tablets 112 may include 10 to 95 wt % NaCl, such as 20 to 90 wt % NaCl, including 30 to 80 wt % NaCl, or 40 to 70 wt % NaCl. In some cases, the osmotic tablets 112 include 30 to 80 wt % NaCl. In certain embodiments, the osmotic tablets 112 include 5 to 90 wt % polymer, such as 10 to 80 wt % polymer, including 20 to 70 wt % polymer, or 30 to 60 wt % polymer. For example, the osmotic tablets 112 may include 5 to 90 wt % Polyox (polyethylene oxide), such as 10 to 80 wt % Polyox, including 20 to 70 wt % Polyox, or 30 to 60 wt % Polyox. In certain cases, the osmotic tablets 112 include 20 to 70 wt % Polyox (polyethylene oxide). In some embodiments, the osmotic tablets 112 are composed of a salt and a polymer, as described above. For example, the osmotic tablets 112 may include 30 to 80 wt % NaCl and 20 to 70 wt % Polyox (polyethylene oxide). In certain instances, the NaCl gives a faster rate of expansion than does the Polyox, though both materials are osmotically active and cause water to be imbibed into the interior of the osmotic driver 110. Because of its low molecular weight, there may be some leakage of NaCl out through the semipermeable membrane 113, whereas because of its high molecular weight, there is substantially no leakage of the Polyox (polyethylene oxide) out through the semipermeable membrane 113. In some instances, a higher NaCl loading (e.g., 80 wt %) gives a shorter duration of dilator expansion than a lower NaCl loading (e.g., 20 wt %).

Adjacent osmotic tablets 112 are separated from one another by way of spacers 114. Spacers 114 as shown in FIG. 10 are annularly (e.g., donut) shaped having an inner opening that is large enough to slide over axial member 101 coated with inner membrane 111. Spacers 114 may be composed of a compressible material such as an elastomer or polymeric foam. By separating the solid osmotic tablets 112 with compressible spacers 114, the central portion of the dilator 100 can accommodate bending at those points where the compressible spacers 114 are positioned. Suitable compressible materials for use in spacers 114 include thermoplastic polyether polyurethanes, such as, but not limited to, Tecophilic® as supplied by the Lubrizol Corporation, Wickliffe, Ohio, e.g., in grades HP-93A-100 and HP-60D-60, or thermoplastic polyester elastomers, such as, but not limited to, Hytrel® as supplied by Dupont™, Wilmington, Del., or closed cell foam forms of such elastomeric polymers.

Optionally, and as shown in FIG. 10, the dilator 100 can also have compressible members 115 positioned between the distal-most osmotic tablet 112 and the distal tip 104 and between the proximal-most osmotic tablet 112 and the proximal anchor 105 in order to create additional bend points along the length of the dilator 100. Compressible members 115 additionally encapsulate the proximal and distal ends of osmotic driver 110 to axially contain the osmotic tablets during expansion of the dilator and direct the expansion in a radial direction. The compressible members 115 can be formed of the same or similar materials, e.g., a compressible material such as an elastomer or polymeric foam, as the spacers 114. Thus, in the dilator 100 shown in FIG. 10 having an osmotic driver 110 comprised of five solid and relatively non-compressible osmotic tablets 112, which tablets are separated by four compressible spacers 114, has six places along the length of the osmotic driver 110 which can accommodate at least some degree of bending, which bending is accommodated by the malleable nature of axial member 101 in combination with the relatively flexible and stretchable (e.g., elastic) nature of membranes 111 and 113.

In some instances, the degree of bending will depend upon the relative compressibility of the spacers 114 as well as their thickness and/or the distance between adjacent solid osmotic tablets 112. A greater distance between adjacent osmotic tablets 112, and/or more compressible spacers 114 will accommodate a greater degree of bending compared to a lesser distance between adjacent osmotic tablets 112, and/or less compressible spacers 114.

Although it is possible to have no spacers and simply space the osmotic tablets a sufficient distance apart from one another in order to accommodate bending of the dilator 100, such a configuration introduces a void within the confines of the driver 110, which may cause a significant time delay in the start of dilator 110 expansion compared to those embodiments where the spacers 114 occupy such voids.

In certain embodiments, the dilator has from 3 to 8 of the bendable segments (e.g., the bendable/compressible spacers, including the bendable/compressible end caps, as described herein) and from 2 to 7 of the non-bendable segments (e.g., the osmotic tablets as described herein). In certain embodiments, the dilator has from 5 to 7 of the bendable segments and from 4 to 6 of the non-bendable segments. In some instances, the dilator has a greater number of bendable segments than the number of non-bendable segments. For example, the dilator may include one additional bendable segment than the number of non-bendable segments. For instance, in some embodiments, the dilator includes 5 non-bendable segments (e.g., the osmotic tablets as described herein) and 6 bendable segments (e.g., the bendable/compressible spacers, including the bendable/compressible end caps, as described herein), see, e.g., FIG. 10.

Once dilator 100 is inserted into a frontal sinus recess 12, water from the patient's body permeates through the membrane 113 by osmosis and forms a solution of the salt or sugar and hydrates the osmopolymer in the osmotic tablets 112, thereby causing the osmotic tablets 112 to expand. As water imbibes in, the volume of the osmotic tablets 112 increases. In addition, due to its elastic nature, the membrane 113 also expands to accommodate the increased volume of the osmotic tablets 112. The rate of water permeation can be controlled by controlling the composition, thickness and porosity of the membrane 113, in combination with the osmotic activity of the tablets 112. In certain embodiments of the devices disclosed herein, the membrane 113 composition, thickness and porosity are controlled to achieve expansion of the osmotic tablets 112 over a period of 0.5 hours or more, such as 1 hour or more, including 2 hours or more, or 3 hours or more, or 4 hours or more. In other embodiments of the devices disclosed herein, the membrane 113 composition, thickness and porosity are controlled to achieve expansion of the osmotic tablets 112 over a period of 4 hours or more. In this way, the rapid expansion and the resulting pain experienced by the patient during balloon catheter sinuplasty procedures may be substantially reduced and/or avoided.

Referring now to FIGS. 11 and 12, there is shown the dilator 100 in an expanded configuration after it has been in place within a frontal sinus recess 12. The cross-sectional view of FIG. 12 relates to FIG. 11 in the same way as the cross-sectional view of FIG. 10 relates to FIG. 7. As can be seen by a comparison of FIGS. 10 and 12, the volume of the osmotic tablets 112 has expanded due to the imbibed water and the elastic semipermeable membrane 113 has expanded to accommodate this increased volume. In this way, the diameter of the osmotic tablets 112 has increased and, when in place within the frontal sinus recess 12, exerts an outward radially expansive force thereon, causing the adjacent portions of the frontal sinus recess to dilate.

Optionally, the dilator 100 may be configured to release a drug while in place within the frontal sinus recess 12. The dilator 100 may be preloaded with a drug. For example a drug can be loaded into the osmotic tablets 112. In certain instances, the drug is water soluble and thereby acts itself as an osmotic agent. In some cases, the drug is present in a coated layer upon the surface of the dilator 100. The coated layer may include a blend of film former polymer mixed with the drug. In yet another embodiment, the drug is blended directly into membrane 113 which drug elutes in situ directly to tissues lining the frontal sinus recess. Alternatively, a drug may be coated onto the outer surfaces of dilator 100 by the physician immediately before use. For example, the dilator 100 can be sprayed, dipped or coated with a drug solution or gel formulation that includes a drug prior to placement of dilator 100 within the patient. The drug added to dilator 100 may be selected from antibiotics, anti-inflammatory drugs, anesthetics (e.g., local anesthetics), analgesics (e.g., locally acting analgesics), drugs that reduce bleeding (e.g., vasoconstrictors), combinations thereof, and the like. In certain embodiments, antibiotics include levofloxacin, moxifloxacin, amoxicillin, clavulanic acid, clarithromycin, azithromycin, cefuroxime, ciprofloxacin, salts thereof and combinations thereof and the like. In some instances, anti-inflammatory drugs include budesonide, mometasone, prednisone, methylprednisolone, dexamethasone, triamcinolone acetonide, salts thereof and combinations thereof and the like. In some cases, local anesthetics include lidocaine, bupivacaine, ropivacaine, tetracaine, salts thereof and combinations thereof and the like. In certain embodiments, locally acting analgesics include: acetaminophen; Cox-2 inhibitors, such as celecoxib and rofecoxib and the like; NSAIDS such as diclofenac, ibuprofen, ketoprofen, naproxen, piroxicam, aspirin and the like; opioids such as morphine; opioid agonists such as tramadol and the like. In certain embodiments, vasoconstrictors include oxymetazoline, epinephrine, tranexamic acid, salts thereof, combinations thereof, and the like. In certain instances, the drug reservoirs may include a combination of drugs, such as a combination of an NSAID, an anti-inflammatory drug and a vasoconstrictor. For example, the drug may include OMS103HP (Omeros Corp., Seattle, Wash.), which includes an NSAID (ketoprofen), an anti-inflammatory drug (amitriptyline) and a vasoconstrictor (oxymetazoline).

In certain embodiments, insertion device 200 includes a light source, which in some instances is a directional light source, such as a low energy laser. Alternatively, the light source may be distant from the insertion device 200 but the light is transmitted from the source to the nasal cavity via a light-transmitting conduit, e.g., a fiber optic cable, within or on the insertion device 200. The light source may emit light into the lumen of tube 202 using a light directing means and a light-reflecting interior surface of tube 202. In some embodiments, dilator 100 and the arm 207 that the dilator is actuated by are also constructed of light transmitting and/or translucent materials so that the light from the light source causes at least portions of the dilator 100 to become illuminated. The illumination may have sufficient intensity so that the emitted light can be seen through the patient's facial tissue. The position of the illuminated dilator 100 may help the physician to correctly position the dilator in the frontal sinus recess. Alternatively, the dilator 100 described herein may be placed using an illuminated guide wire that extends through the tube 202 and/or through arm 207 of the insertion device 200, and optionally through the internal lumen of axial member 101 of the dilator 100.

In some embodiments, insertion device 200 may be configured to include a camera positioned near the distal end of the tube 202 in order to assist in visualizing the stenotic sinus opening or nasal cavity. In some instances, the camera may be positioned on the exterior surface of the tube 202 and, for example, electrically coupled to a monitor via an electrical wire extending along or within the tube 202.

The insertion device 200, or components thereof, may be configured for one time use (i.e., disposable) or may be re-usable, e.g., where the components are configured to be used two or more times before disposal, e.g., where the device components are sterilizable.

Additional aspects of the insertion devices and methods for use are also described in U.S. Application Publication Nos. 2012/0053567 and 2012/0053404, and U.S. Application Publication No. 2013/0231693, the disclosures of each of which are incorporated herein by reference.

In certain embodiments, the osmotic driver 110 is configured to begin expanding upon insertion of the dilator 100 into the stenotic frontal sinus opening and or recess of the subject. In some instances, the driver 110 is configured to begin expanding within seconds or minutes after insertion of the dilator 100 into the stenotic frontal sinus opening and/or recess. In some cases, the driver 110 is configured to begin expanding in 60 min or less, such as 45 min or less, or 30 min or less, including 10 min or less, or 5 min or less, such as 1 min or less, after insertion of the dilator into the stenotic frontal sinus opening and/or recess. In some instances, the driver 110 is configured to continue to expand for a certain period of time after the dilator 100 has been inserted into the stenotic frontal sinus opening and/or recess of the subject. For example, the driver 110 may be configured to continue to expand for 30 min or more, such as 45 min or more, including 60 min or more, or 90 min or more, 120 min or more, or 180 min or more, or 240 min or more, or 300 min or more after the dilator 100 has been inserted into the stenotic frontal sinus opening and/or recess of the subject.

In certain embodiments, the driver 110 takes a certain amount of time to expand the expandable portion from the non-expanded configuration to the expanded configuration. For instance, in some cases the driver 110 is configured to expand the expandable portion from the non-expanded configuration to the expanded configuration over a period of 0.5 hours or more, such as 1 hour or more, or 2 hours or more, or 4 hours or more, or 6 hours or more, or 8 hours or more, or 10 hours or more, or 12 hours or more, or 24 hours or more, or 48 hours or more, or 72 hours or more, etc. In some instances, the driver 110 is configured to expand the expandable portion from the non-expanded configuration to the expanded configuration over a period of 24 hours or less, such as 12 hours or less, or 10 hours or less, or 8 hours or less, or 6 hours or less, or 4 hours or less, or 2 hours or less, 1.5 hours or less, or 1 hours or less, or 0.5 hours or less. As such, in certain instances, the driver 110 is configured to expand the expandable portion from the non-expanded configuration to the expanded configuration over a period ranging from 0.5 hours to 24 hours, such as 0.5 hour to 12 hours, including 0.5 hour to 10 hours, or 1 hour to 8 hours, or 1 hour to 6 hours, or 1 hour to 4 hours, or 1 hour to 2 hours.

In certain embodiments, the driver 110 is configured to expand the expandable portion to a diameter of 10 mm or less, such as 9 mm or less, or 8 mm or less, or 7 mm or less, or 6 mm or less, or 5 mm or less, or 4 mm or less, or 3 mm or less, or 2 mm or less, or 1 mm or less. In some cases, the driver 110 is configured to expand the expandable portion to a diameter of 7 mm or less.

Embodiments of the presently disclosed dilators include an expandable portion, which in the FIGS. 10-12 embodiment includes the osmotic tablets 112 and the outer elastic semipermeable membrane 113. The expandable portion is configured to expand from a non-expanded configuration to an expanded configuration. In certain embodiments, the expandable portion is configured to expand in size from a non-expanded configuration to an expanded configuration. The expandable portion may be configured to expand in size without significantly increasing in volume, such as by stretching in one or more dimensions from the non-expanded configuration. The expandable portion may be positioned peripherally around the driver 110. For instance, the expandable portion may be disposed on an exterior surface of the driver 110. In these embodiments, expansion of the underlying driver 110 expands the expandable portion from its non-expanded configuration to its expanded configuration.

Aspects of the present disclosure include dilators that have an expandable portion, where the expandable portion includes a membrane. For instance, the expandable portion may be disposed on at least a portion of the driver 110, such as surrounding the outer surface of the driver 110. The membrane may be an elastic membrane, such that the membrane is configured to expand from the non-expanded configuration to the expanded configuration, as described herein. In certain instances, the membrane is a semipermeable membrane. By “semipermeable” is meant a membrane that is permeable to solvent but not significantly permeable to solute across a concentration gradient, such as a membrane that allows solvent (e.g., water) molecules to pass through the membrane by osmosis from a region of low solute concentration to a region of high solute concentration until a state of dynamic equilibrium is reached. For instance, a semipermeable membrane may be configured to allow water to pass through the membrane by osmosis from a region of low solute concentration (e.g., high water potential) to a region of high solute concentration (e.g., low water potential) until a state of dynamic equilibrium is reached.

In certain embodiments, the expandable portion includes a membrane, where the membrane is an impermeable membrane. By “impermeable” is meant a membrane that is not significantly permeable to solvent or solute. Impermeable membranes do not allow significant amounts of solvent (e.g., water) or solute molecules to pass through the membrane by osmosis even in the presence of a solute concentration gradient across the membrane.

In certain embodiments, membrane 113 includes two membrane layers. In some instances, the membrane layers are in contact with each other and disposed along the entire length of the dilator spanning the distance from proximal tip 104 to distal anchor 105.

In certain embodiments, the dilator includes a hollow axial member 101 that defines an interior lumen of the dilator. The member 101 includes a distal end configured to be in fluid communication with an interior lumen of the paranasal sinus in the subject. In some cases, the axial member 101 may be configured to allow fluid flow between the paranasal sinus in the subject and the nasal cavity when the dilator is positioned within the stenotic frontal recess. In some instances, the axial member 101 is configured to allow fluid and/or air to flow from the paranasal sinus to the nasal cavity of the subject. For example, the axial member 101 may be configured to facilitate drainage of fluid from the frontal sinus in the subject to the nasal cavity when the dilator is positioned within the stenotic frontal recess. In some cases, the axial member 101 may be configured to facilitate the flow of air into and out of the frontal sinus in the subject.

In certain embodiments, the driver 110 is disposed on an exterior surface of the axial member 101. The driver 110 may be disposed on the exterior surface of the axial member 101 at a position between the distal end and the proximal end of the axial member 101. For example, the driver 110 may be positioned between a distal tip at the distal end of the axial member and a proximal anchor at the proximal end of the axial member. As described herein, the expandable portion may be positioned peripherally around the driver 110. Thus, in these embodiments, the driver 110 is disposed between the exterior surface of the axial member and the overlying expandable portion. Expansion of the driver 110 expands the overlying expandable portion from its non-expanded configuration to its expanded configuration.

Aspects of the driver 110 further include embodiments where the driver 110 completely surrounds the axial member 101. The driver 110 may be disposed on the exterior surface of the axial member 101 around the entire periphery of the axial member. In certain embodiments, the driver 110 surrounds the axial member around the central portion of the axial member. In some instances, the driver 110 includes one or more subunits, where each subunit is disposed on the exterior surface of the axial member. The one or more driver subunits may be positioned such that there is a space between the driver subunits.

In certain embodiments, the walls of the axial member are substantially non-collapsible. The walls of the axial member may be substantially non-collapsible, such that the axial member is configured to maintain an opening in the axial member during use of the device. For example, the walls of the axial member may be substantially non-collapsible, such that the axial member is not crushed by the driver 110 during use of the device. In some cases, a non-collapsible axial member maintains substantially the same shape and size during use of the device. For instance, the axial member may maintain substantially the same interior diameter during use of the device. In some instances, the walls of the axial member are substantially non-collapsible, such that pressure exerted on the exterior surface of the axial member by the driver 110 does not significantly decrease the interior diameter of the axial member. As discussed above, the driver 110 may be configured to expand radially outward from the axial member and, as such, the walls of the axial member may be substantially non-collapsible, such that expansion of the driver 110 is directed radially outward away from the substantially non-collapsible walls of the axial member. Expansion of the driver 110 radially outward from the axial member may facilitate dilation of the stenotic frontal recess and/or frontal sinus opening. As described above, a substantially non-collapsible axial member may be flexible and adapted to bend from its original shape. In some instances, a flexible axial member facilitates insertion of the sinus dilator in a sinus ostium or frontal sinus recess.

In some embodiments, the device includes an axial member that includes a semipermeable membrane, a surrounding driver 110, and an overlying expandable portion that includes a semipermeable membrane. In these embodiments, the device may be configured to allow solvent (e.g., water) to pass through both the semipermeable expandable portion membrane by osmosis and through the semipermeable axial member membrane by osmosis. For example, the device may be configured to allow solvent to pass through the semipermeable expandable membrane from the surrounding tissues to the underlying driver 110, and also allow solvent to pass through the semipermeable axial member membrane from an interior lumen of the axial member to the surrounding driver 110.

In other embodiments, the device includes an axial member that includes a semipermeable membrane, a surrounding driver 110, and an overlying expandable portion that includes an impermeable membrane. In these embodiments, the device may be configured to allow solvent (e.g., water) to pass through the semipermeable axial member membrane by osmosis but not allow significant amounts of solvent (e.g., water) to pass through the impermeable expandable portion membrane. For example, the device may be configured to allow solvent to pass through the semipermeable axial member membrane from an interior lumen of the axial member to the surrounding driver 110, but not allow significant amount of solvent to pass through the impermeable expandable portion membrane to the driver 110.

In yet other embodiments, the axial member includes an impermeable material. In some cases, the impermeable material is an impermeable membrane. For instance, the device may include an axial member that includes an impermeable membrane, a surrounding driver 110, and an overlying expandable portion that includes a semipermeable membrane. In these embodiments, the device may be configured to allow solvent (e.g., water) to pass through the semipermeable expandable membrane by osmosis but not allow significant amounts of solvent (e.g., water) to pass through the impermeable axial member membrane. For example, the device may be configured to allow solvent to pass through the semipermeable expandable portion membrane from the surrounding tissues to the underlying driver 110, but not allow significant amount of solvent to pass through the impermeable axial member membrane from the interior lumen of the axial member to the surrounding driver 110.

In some instances, the dilator has a frictional or ridged surface on an exterior surface of the dilator. The frictional surface or ridged may be configured to increase the friction between the exterior surface of the dilator and the surrounding tissues when the dilator is in use. Increasing the friction between the exterior surface of the dilator and the surrounding tissues may facilitate retention of the dilator in the stenotic sinus opening or frontal recess of the subject during use. For example, the frictional surface may have a rough topography that includes an exterior surface shaped as, for example, ridges, washboard, rings, waffle pattern, snow tire pattern, pebble finish, shark skin texture, combinations thereof, and the like.

In certain cases, the dilator includes an adhesive disposed on an exterior surface of the dilator. In some cases, the membrane includes an adhesive. The membrane may be configured such that the adhesive elutes to the external surface of the dilator during use. The adhesive may facilitate retention of the dilator in the stenotic sinus opening or frontal recess of the patient during use. Examples of suitable adhesives include, but are not limited to, carbomer, low molecular weight hydroxypropyl methylcellulose, polyvinyl pyrrolidone, combinations thereof, and the like.

Aspects of the dilator may include a proximal anchor configured to maintain the dilator within the stenotic opening or frontal recess during use. The proximal anchor may be connected to the dilator proximate to the proximal end of the dilator. For example, the proximal anchor may be connected to the dilator proximate to the proximal end of the malleable axial member. In some cases, the proximal anchor is configured to prevent the dilator from being inserted too far or completely into the paranasal sinus of the subject. The proximal anchor may facilitate maintaining the dilator within the stenotic opening or frontal recess for a desired period of time until the dilator is removed from the stenotic opening or frontal recess by the user or a health care professional. In some cases, the proximal anchor has an outside diameter that is greater than the diameter of the dilator. For instance, the proximal anchor may have an outside diameter that is greater than the diameter of the dilator when the dilator is in a non-expanded or expanded configuration.

In some embodiments, the dilator includes a portion configured to facilitate removal of the dilator from the sinus opening or recess. The portion may be configured to allow a removal device to be attached to the dilator. For example, the portion may include a loop, a knob, a tether or a hook. The removal device may include a corresponding structure that allows for attachment of the removal device to the portion of the dilator. In some instances, the dilator includes a loop and the removal device includes a hook. In other embodiments, the dilator includes a hook and the removal device includes a loop. In either embodiment, insertion of the hook into the loop connects the dilator to the removal device and may facilitate removal of the dilator from the sinus opening or recess.

In some cases, the portion configured to facilitate removal of the dilator may protrude from the dilator to facilitate connection of the removal device to the portion of the dilator. The portion may be disposed at or near the proximal end of the dilator to facilitate removal of the dilator from the sinus opening or recess. For example, the portion may be disposed on the proximal anchor at the proximal end of the dilator. In certain cases, the portion may be connected to the malleable axial member proximate to the proximal end of the dilator. For example, the proximal end of the axial member may include a knob that can be grasped using forceps.

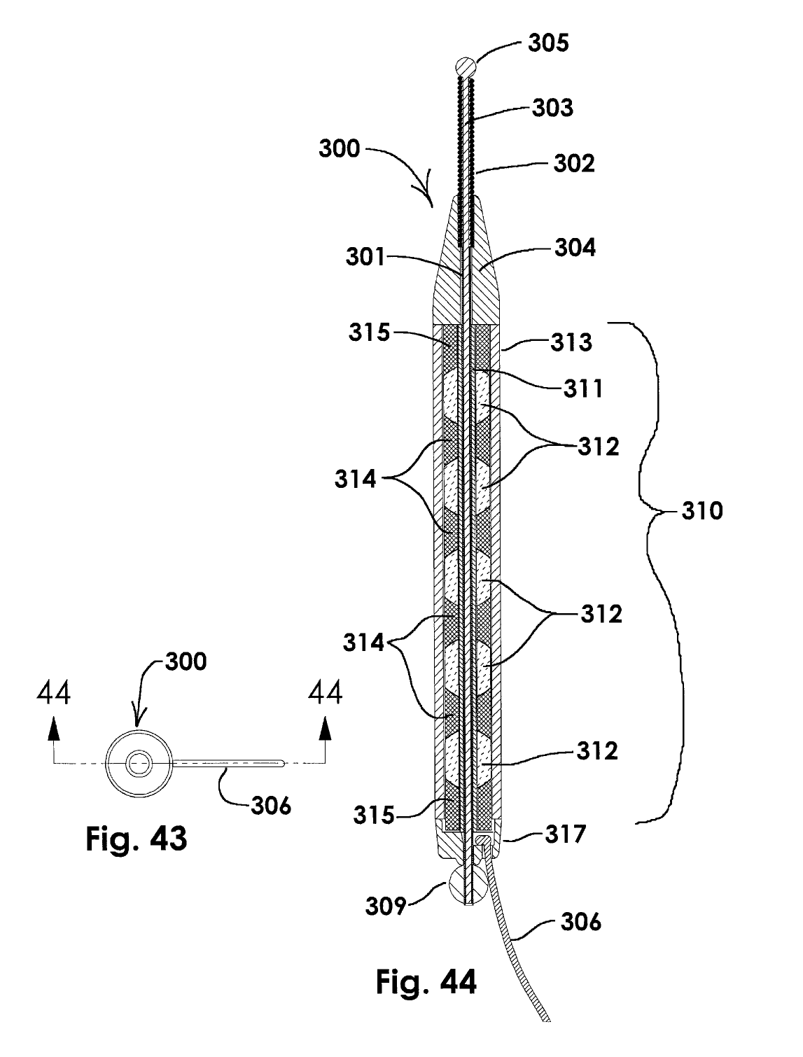

Referring now to FIGS. 43 to 46, there is shown an alternate embodiment of a dilator 300 for dilating a stenotic frontal sinus opening or frontal sinus recess of a patient. FIGS. 43 and 44 show the dilator 300 in a non-expanded configuration which is the configuration at the time the dilator 300 is first positioned within a frontal sinus recess 12. Dilator 300 has a tapered distal tip 304 to facilitate insertion into a stenotic frontal sinus recess 12. Distal tip 304 is composed of a soft elastomeric material that is easily bent in a direction that is approximately perpendicular to the axis of the dilator 300.

Unlike dilator 100, dilator 300 has no proximal anchor. Other embodiments of dilator 300 may include a proximal anchor as described herein. The tether 306 may be used to ensure that the dilator 300 is not inhaled or swallowed, or otherwise lost, should the dilator 300 become dislodged from frontal sinus recess 12. The tether may also be used to pull the dilator 300 out of the frontal sinus recess 12 after the frontal sinus recess 12 has been dilated. Alternatively, the healthcare provider (e.g., surgeon) can grab knob 309 at the proximal end of the dilator 300 with forceps in order to remove the dilator 100 from frontal sinus recess 12.

Like dilator 100 described above, dilator 300 also has a malleable axial member 301 running through substantially the entire length thereof. A spring 302 comprised of a coiled wire abuts the distal end of axial member 301 and extends out of the distal end of tip 304. Spring 302 is an extension type spring with adjacent coils touching one another, although a compression type spring having a gap between adjacent coils can also be used. The spring 302 is flexible and can be flexed in a direction that is roughly perpendicular to the axis of the spring. Extending within the bore of axial member 301 and coiled spring 302 is a wire 303 made of shape memory metal, e.g., a nickel-titanium alloy such as nitinol. Wire 303 has a bulbous distal end 305 that is formed by arc welding a short length of the wire sufficient to achieve a roughly spherically-shaped end having a diameter that is about 3 to 4 times that of the wire diameter. The length of wire 303 extending beyond the distal end of tip 304 is about 5 to 10 mm. The bulbous shaped end 305, together with the bendable spring 302 and the bendable tip 304, form a flexible finger which helps the healthcare provider (e.g., surgeon) guide the dilator 300 into the frontal sinus recess.

Adjacent osmotic tablets 312 are separated from one another by way of spacers 314. Spacers 314 are annularly (e.g., donut) shaped having an inner opening that is large enough to slide over axial member 301 coated with inner membrane 311. Spacers 314 may be composed of a compressible material such as an elastomer or polymeric foam. By separating the solid osmotic tablets 312 with compressible spacers 314, the central portion of the dilator 300 can accommodate bending at those points where the compressible spacers 314 are positioned. Suitable compressible materials for use in spacers 314 are the same as those used for spacers 114 in dilator 100.

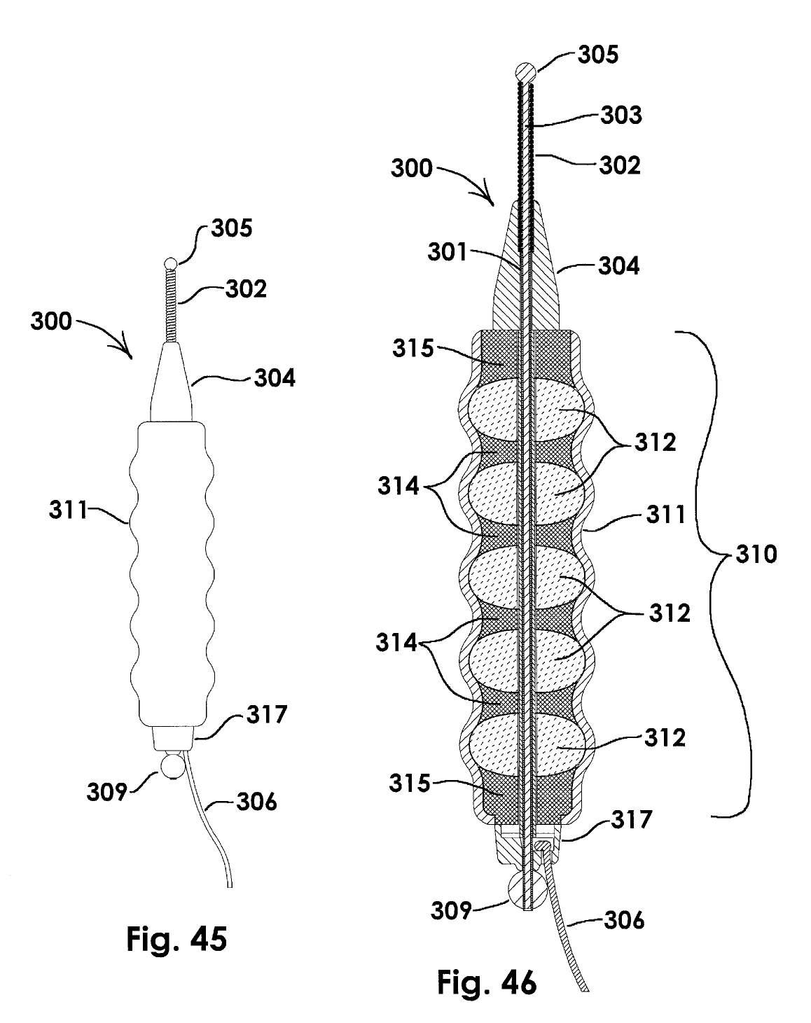

Referring now to FIGS. 45 and 46, there is shown the dilator 300 in an expanded configuration after it has been in place within a frontal sinus recess 12. The cross-sectional view of FIG. 46 relates to FIG. 45 in the same way as the cross-sectional view of FIG. 10 relates to FIG. 7. As can be seen by a comparison of FIGS. 44 and 46, the volume of the osmotic tablets 312 has expanded due to the imbibed water and the elastic semipermeable membrane 313 has expanded to accommodate this increased volume. In this way, the diameter of the tablets 312 has increased and, when in place within the frontal sinus recess 12, exerts an outward radially expansive force thereon, causing the adjacent portions of the frontal sinus recess to dilate.

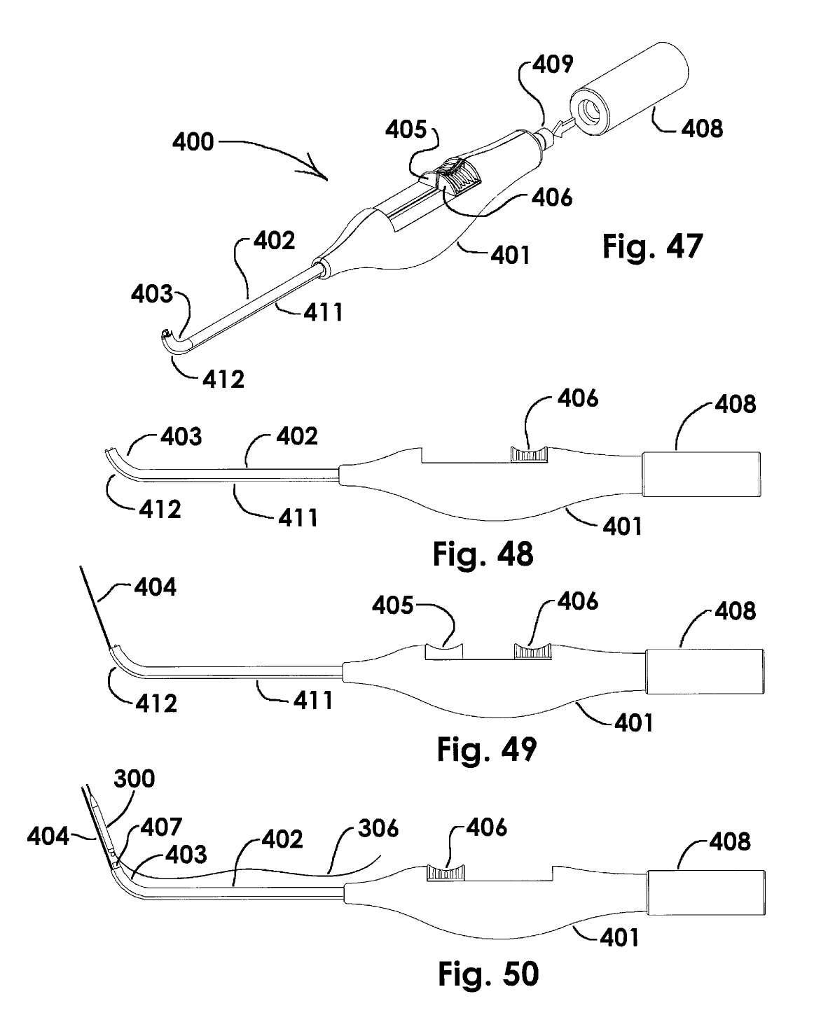

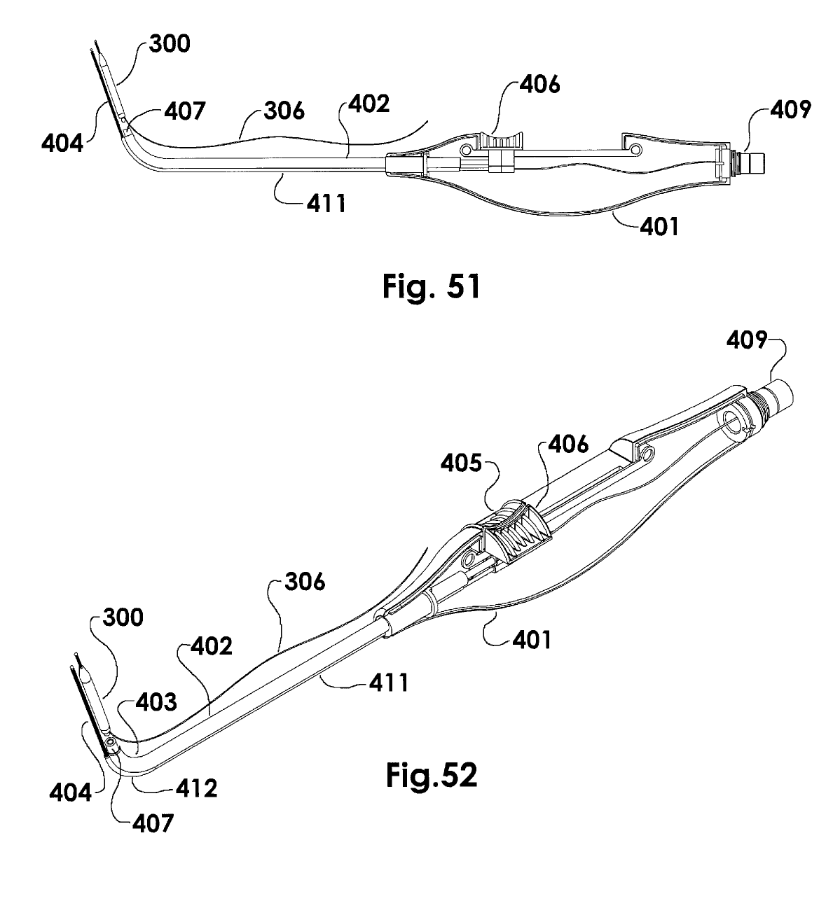

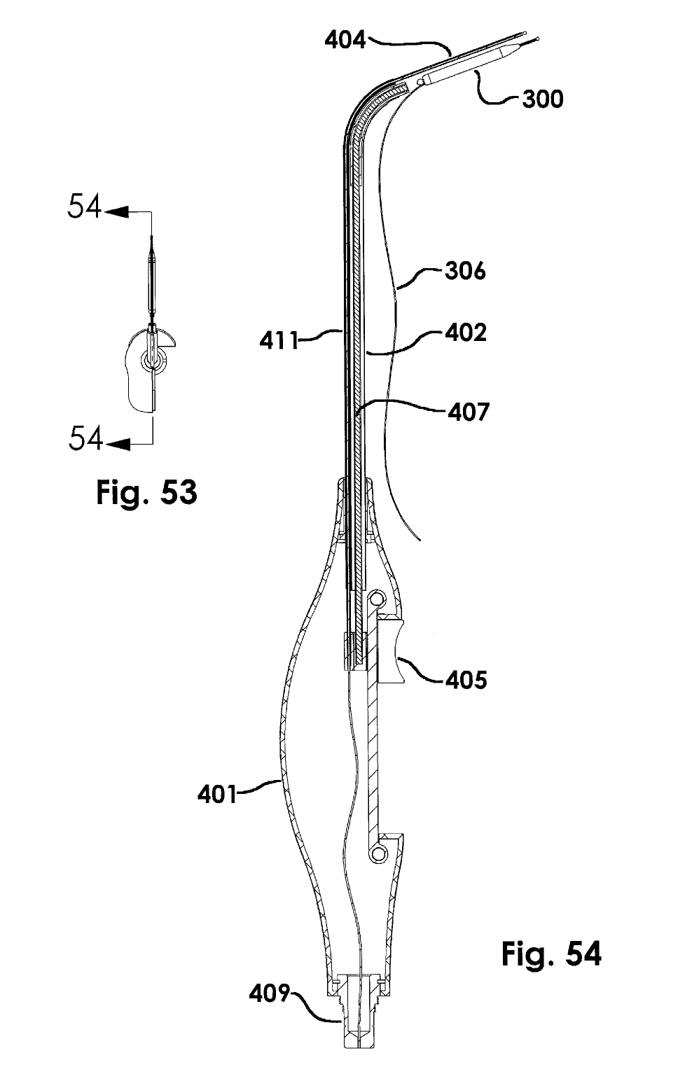

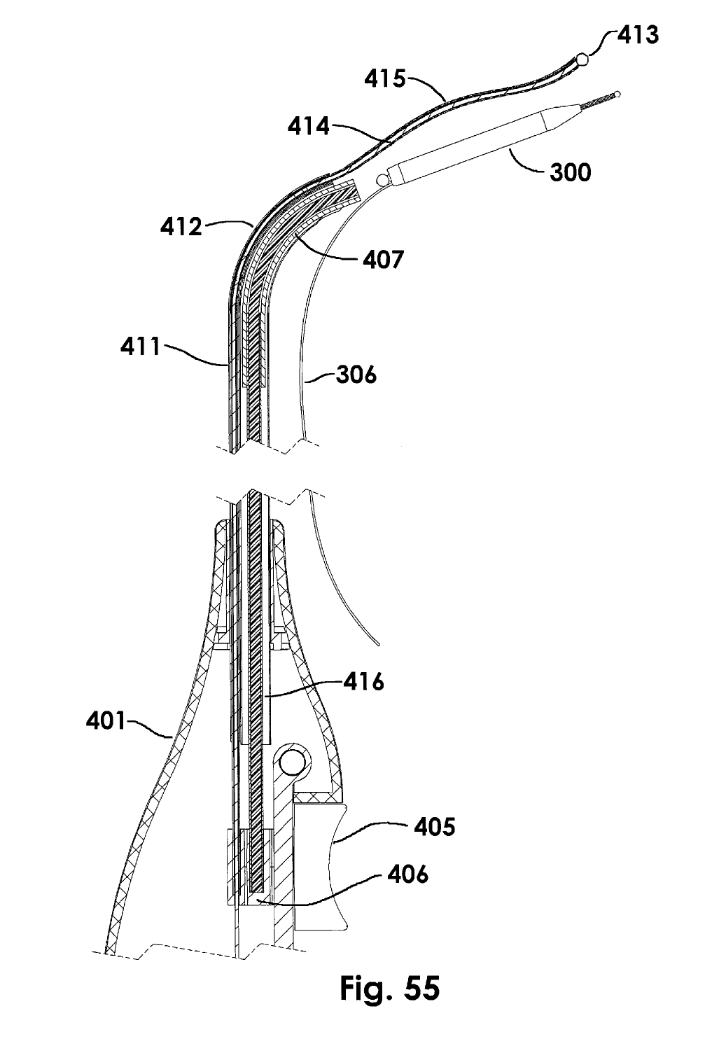

Insertion of a frontal sinus dilator 300 into a stenotic frontal sinus recess will now be described while referring to FIGS. 47 to 54. The insertion device 400 includes a handle 401, a first tube 402 having an angled distal end 403, and a second tube 411 having an angled distal end 412. A light source 408 connects onto the proximal end of handle 401. One suitable light source 408 is a light emitting diode (LED), such as a portable battery operated light emitting diode (LED), for example a light source sold by Optim LLC of Sturbidge, Mass. which connects to handle 401, or for example via a light post fitting, such as a Storz type light post fitting 409 (Part No. STLP643 sold by Endoscope Replacement Parts, Inc. of Newberry, Fla.). Extending out of the angled distal end 412 is a light wire 404. Light wire 404 includes a lens 413, a light-transmitting flexible optical fiber 414, a coil 415, and a supporting hypotube 416 (see FIG. 55). The lens 413 is an optically clear drop of urethane acrylate adhesive that attaches the optical fiber 414 to the distal end of coil 415. The optical fiber 414 runs through the insertion device 400 and is attached at its proximal end to the lightpost fitting 409. The flexible optical fiber 414 is composed of poly methyl methacrylate (PMMA) and has a diameter ranging from 0.2 to 0.6 mm. The distal portion of the fiber 414 is covered by a flexible stainless steel closed wound coil 415. The proximal end of the optical fiber 414 is encased in a rigid stainless steel hypotube 416. The hypotube 416 and the optical fiber 414 are attached to the actuator 405 using adhesive. The optical fiber 414 extends proximally through handle 401 and attaches to the lightpost fitting 409. Positioned proximally within the lightpost fitting 409 is an optical urethane acrylate lens which funnels light from the light source 408 into the optical fiber 414. In this manner, a conventional light source, such as a portable light source 408 or a non-portable light conducting cable (not shown in the figures) can be connected through conventional means to fitting 409 in order to cause the distal end of light wire 404 to become illuminated. The illumination provided by light wire 404 helps the healthcare provider (e.g., surgeon) determine the position of the light wire and the distal end 403 when the device is in place within the patient's head, thereby providing feedback on whether the insertion device 400 is correctly positioned for deployment of the dilator 300 into the frontal sinus recess.

Also as shown in FIGS. 47 and 48, the distal tip of dilator 300 just extends out of the angled distal end 403. Slidably mounted on the handle 401 are a pair of actuators; a light wire advancing actuator 405 and a dilator deployment actuator 406. Like the dilator 100 positioned within the tube 202 described above, initially the dilator 300 is also positioned within the tube 402 of insertion device 400. Once the healthcare provider (e.g., surgeon) has the insertion device 400 positioned in the patient's nasal cavity such that the tip of angled distal end 403 is near the start of the frontal sinus recess 12, the healthcare provider (e.g., surgeon) may then slide actuator 405 in a distal direction, which causes the light wire 404 to advance in a distal direction, where it extends beyond the opening in angled distal end 412, as shown in FIG. 49. In the context of the nasal cavity, extending the light wire 404 from the position shown in FIG. 48 to the position shown in FIG. 49 causes the light wire 404 to extend through the frontal sinus recess 12 and into the frontal sinus cavity (FS). Once the light wire 404 is extended through the frontal sinus recess 12, the healthcare provider (e.g., surgeon) may slide actuator 406 in a distal direction, which causes arm 407 to move distally and push the dilator 300 in a distal direction. In some cases, an electrical circuit can be created to actuate the mechanical translation of the arm 407, such as, for example by electrical actuation of a solenoid element. Arm 407 may be flexible so that it can bend while advancing through the angled distal end 403. The distally advancing arm 407 pushes the dilator 300 in a distal direction, causing the dilator 300 to be pushed out of the distal end 403, as shown in FIGS. 50 to 52. In the context of the nasal cavity, sliding the actuator 406 in a distal direction causes the dilator 300 to be pushed into the frontal sinus recess 12. Once the dilator is in place within the frontal sinus recess 12, the healthcare provider (e.g., surgeon) may pull the insertion device 400 out of the patient's nasal cavity, leaving the dilator 300 in place within the frontal sinus recess 12. Optionally, the light wire 404 can be retracted back into the tube 411 and distal end 412 by sliding the actuator 405 in a proximal direction before the insertion device 400 is pulled out of the nasal cavity.

The light wire 404 in insertion device 400 differs from guidewire 204 in insertion device 200 described above in the sense that the light wire 404 and the dilator 300 are not coaxial, so the dilator 300 is not slid over the light wire into position in the frontal sinus recess. Instead, the lighted wire is adjacent to the dilator 300. In addition, guidewire 204 is not light-conducting, whereas light wire 404 is light-conducting. As best shown in FIGS. 52 and 54, the proximal end of light wire 404 connects to fitting 409.

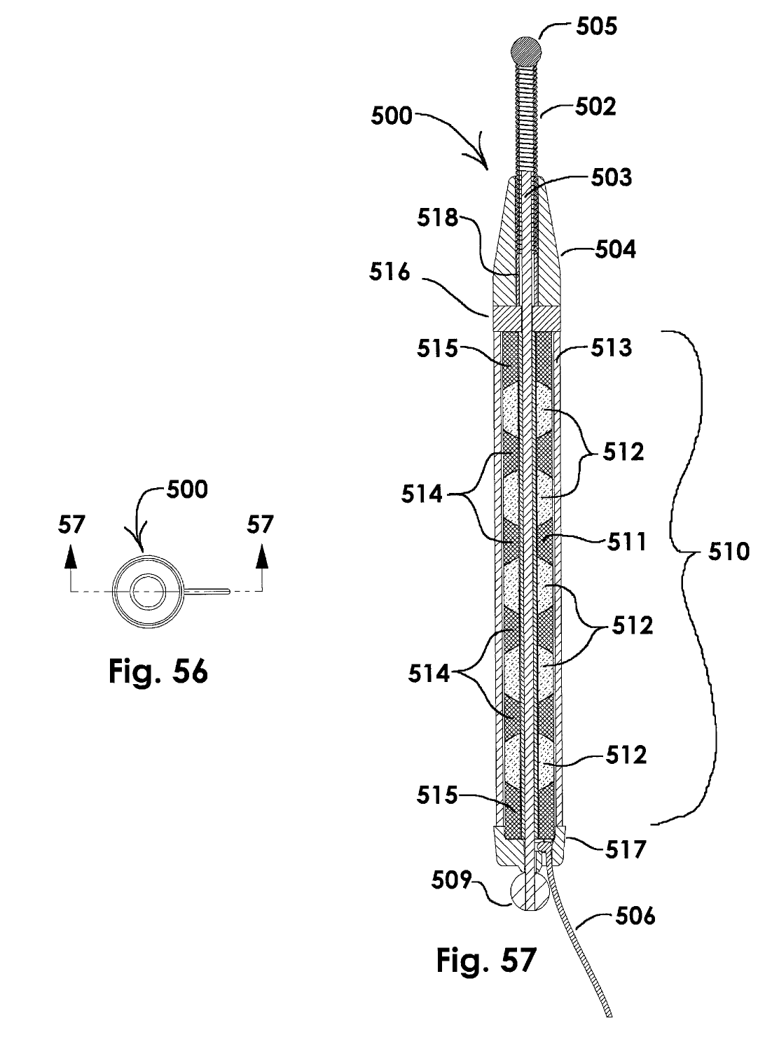

Referring now to FIGS. 56 and 57, there is shown an alternate embodiment of a dilator 500 for dilating a stenotic frontal sinus opening or frontal sinus recess of a patient. FIGS. 56 and 57 show the dilator 500 in a non-expanded configuration which is the configuration at the time the dilator 500 is first positioned within a frontal sinus recess 12. Dilator 500 differs from dilator 300 in that a single malleable axial rod 503 is used in place of the malleable axial member 301 and malleable wire 303.

Similar to dilator 300, dilator 500 also has a tapered distal tip 504 to facilitate insertion into a stenotic frontal recess 12 and a tether 506 and knob 509 to facilitate removal of the dilator 500 from the sinus recess and nasal cavity. Distal tip 504 is composed of a soft elastomeric material that is easily bent in a direction that is approximately perpendicular to the axis of the dilator 500. Like dilator 300, dilator 500 also has a spring 502 comprised of a coiled wire extending out of the distal end of tip 504. Spring 502 is an extension type spring with adjacent coils touching one another, although a compression type spring having a gap between adjacent coils can also be used. The spring 502 is flexible and can be flexed in a direction that is roughly perpendicular to the axis of the spring. Spring 502 has a bulbous distal end 505 that is formed by welding or gluing a roughly spherically-shaped metal piece having a diameter that is about 2 times the inner diameter of spring 502. The length of spring 502 extending beyond the distal end of tip 504 is about 5 to 10 mm. The bulbous shaped end 505, together with the bendable spring 502 and the bendable distal tip 504, form a flexible finger which helps the healthcare provider (e.g., surgeon) guide the dilator 500 into the frontal sinus recess.

Extending within the center of osmotic driver 510 is a malleable rod 503 made of stainless steel, such as annealed stainless steel, or a shape memory metal, e.g., a nickel-titanium alloy such as nitinol. Covering rod 503 in the region of the osmotic driver 510, e.g., threaded over the rod 503 and inner membrane 511, are a series of osmotic tablets 512, separated by flexible spacers 514 and also flexible end members 515. Both the osmotic tablets 512 and spacers 514 are annularly (e.g., donut) shaped having an inner opening that is large enough to slide over axial rod 503 covered with inner membrane 511. Spacers 514 may be composed of a compressible material such as an elastomer or polymeric foam. By separating the solid osmotic tablets 512 with compressible spacers 514, the central portion of the dilator 500 can accommodate bending at those points where the compressible spacers 514 are positioned. Suitable compressible materials for use in spacers 514 and members 515 are the same as those used for spacers 314 and members 315 in dilator 300 described above. Dilator 500 also includes an annular disk 516 and end cap 517, both made of relatively rigid plastic. Disk 516 is fixedly attached to rod 503 by way of a metal sleeve 518, positioned distally to disk 516, which sleeve is crimped onto rod 503. End cap 517 abuts against knob 509 which knob is fixedly attached to rod 503 via welding. Since rod 503 is non-stretchable, disk 516 cannot move distally and end cap 517 cannot move proximally. Hence, disk 516 and end cap 517 function to prevent the osmotic driver 510 from expanding in an axial direction, thereby facilitating the expansion of driver 510 and dilator 500 in a radial direction.

In use, dilator 500 expands in a similar manner and shape as dilators 100 and 300 described above. Water from the patient's body is absorbed through the elastic semipermeable membrane 513 via osmosis until the dilator 500 reaches an expanded configuration substantially similar to that shown for dilator 300 in FIGS. 45 and 46.

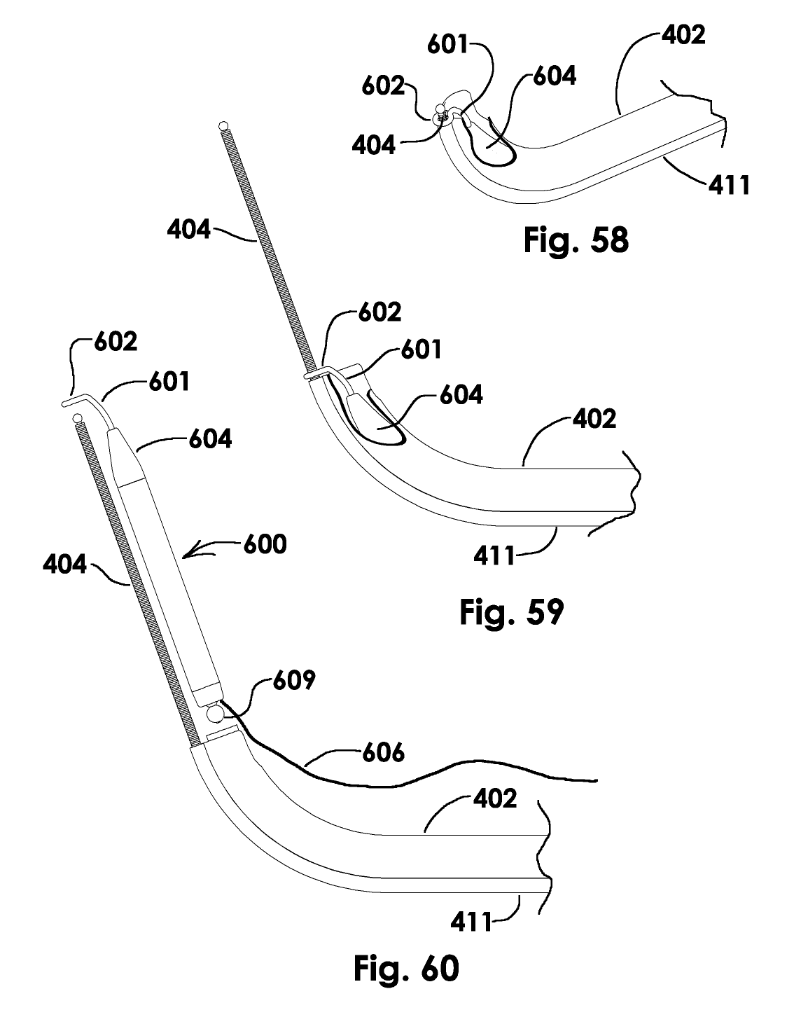

Referring now to FIGS. 58 to 60, there is shown an alternate embodiment of a dilator 600 for dilating a stenotic frontal sinus opening or frontal sinus recess of a patient. FIGS. 58 and 59 show the dilator 600 in a non-expanded configuration while still loaded into the distal end of an insertion device 400, before being positioned within a frontal sinus recess 12.

Dilator 600 differs from dilator 500 in that a single malleable or flexible wire 601, which extends out of the distal end 604 and made of stainless steel, such as annealed stainless steel (e.g., flexible annealed steel), or a shape memory metal, e.g., a nickel-titanium alloy such as nitinol, is used in place of the spring 502. The distal portion of wire 601 ends in a loop 602 that is big enough to slide over light wire 404. In certain embodiments, when wire 601 is a malleable wire, then wire 601 can extend axially through the dilator as described above. In certain embodiments, when wire 601 is a flexible wire, then wire 601 may extend from distal tip in place of spring 502.

Similar to dilator 500, dilator 600 also has a tapered distal tip 604 to facilitate insertion into a stenotic frontal sinus recess 12 and a tether 606 and knob 609 to facilitate removal of the dilator 600 from the sinus recess and nasal cavity.

Once the healthcare provider (e.g., surgeon) has the insertion device 400 positioned in the patient's nasal cavity such that the tip of angled distal end is near the start of the frontal sinus recess 12, the healthcare provider (e.g., surgeon) may then slide actuator 405 in a distal direction, which causes the light wire 404 to advance in a distal direction, where it extends beyond the opening in angled distal end 412, as shown in FIG. 59. In the context of the nasal cavity, extending the light wire 404 from the position shown in FIG. 58 to the position shown in FIG. 59 causes the light wire 404 to extend through the frontal sinus recess 12 and into the frontal sinus cavity (FS). Once the light wire 404 is extended through the frontal sinus recess 12, the healthcare provider (e.g., surgeon) may slide actuator 406 in a distal direction, which causes arm 407 to move distally and push the dilator 600 in a distal direction, and out of the distal opening of tube 402. In the context of the nasal cavity, extending the dilator 600 from the position shown in FIG. 59 to the position shown in FIG. 60 causes the dilator 600 to be pushed into the frontal sinus recess 12.

In use, dilator 600 expands in a similar manner and shape as dilators 100, 300 and 500 described above. Water from the patient's body is absorbed through the outer elastic semipermeable membrane via osmosis until the dilator 600 reaches an expanded configuration substantially similar to that shown for dilator 300 in FIGS. 45 and 46.

Additional aspects of the devices and methods for dilating a stenotic opening of a paranasal sinus in a subject are also described in U.S. Patent Publication Nos. 2012/0053567, 2012/0053404, and 2013/0231693, the disclosures of each of which are incorporated herein by reference.

### Kits

Also provided are kits for use in dilating a stenotic paranasal sinus opening, such as a frontal sinus recess, where the kits may include one or more of the sinus dilators as described herein and an insertion device for inserting the dilator into the frontal sinus recess. As such, a kit may include an insertion device, and may further include one or more sinus dilators. In some instances, a sinus dilator may be provided in the kit as preloaded on the insertion device. In some instances, the sinus dilator may be provided in the kit as decoupled from the insertion device.

In certain embodiments, the kit may include a packaging configured to contain the insertion device and/or sinus dilators. The packaging may be a sealed packaging, such as a sterile sealed packaging. By “sterile” is meant that there are substantially no microbes (such as fungi, bacteria, viruses, spore forms, etc.) inside the sealed packaging. In some instances, the packaging may be configured to be sealed, e.g., a water vapor-resistant packaging, optionally under an air-tight and/or vacuum seal.

In some instances, the kit may further include additional components, such as fluid sources (e.g., water sources, saline solution sources, drug solution sources, etc.), connective tubing, guide wire, light source, power source (e.g., a battery), monitor, etc., which may find use in practicing the subject methods. The drug may be provided in a separate container, such as a syringe, vial, bottle, etc., such that the drug may be filled into a drug reservoir of the insertion device prior to insertion into the stenotic opening or recess. The drug sources may be adapted to couple with the insertion device, such as a cartridge that is coupled to a receiving slot in the insertion device, or such as a container that is coupled to a port on the insertion device via tubing.

Furthermore, the kit may further include one or more additional or different interfaces for coupling different sizes or types of dilators to the insertion device. Such may be desirable where the kit includes sinus dilators of different sizes and/or types, or for convenience for sanitation purposes. Various components may be packaged as desired, e.g., together or separately.

In addition to above mentioned components, the subject kits may further include instructions for using the components of the kit to dilate a paranasal sinus opening, such as a frontal sinus recess. The instructions for practicing the subject methods may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, DVD, Blu-Ray, computer-readable memory (e.g., flash memory), etc. In yet other embodiments, the actual instructions are not present in the kit, but means for obtaining the instructions from a remote source, e.g. via the Internet, are provided. An example of this embodiment is a kit that includes a web address where the instructions can be viewed and/or from which the instructions can be downloaded. As with the instructions, the means for obtaining the instructions is recorded on a suitable substrate.

### EXAMPLES

### Example 1

A batch of stainless steel (Grade 304; iron matrix containing <0.08% C, 17.5-20% Cr, 8-11% Ni, <2% Mn, <1% Si, <0.045% P and <0.03% S) hypotubes having a nominal inside diameter of 0.5 mm (0.020 inch) and an outside diameter of 0.6 mm (0.025 inch) of were cut into sections having a length of 30 cm (1 ft). The cut sections were annealed at one of five different conditions to produce five different types annealed tube sections.

In the first type, the sections were heated in a vacuum for one hour at 1200±25° F. (650±5° C.). The temperature was then ramped to 1650±25° F. (900±5 C.) and held at that temperature for 25 minutes. Next, the tube sections were quenched in a current of nitrogen gas until the chamber was cooled to 100° F. (40° C.). In the second type, the sections were heated in a vacuum for one hour at 1200±25° F. (650±5° C.). The temperature was then ramped to 1750±25° F. (955±5° C.) and held at that temperature for 25 minutes. Next, the tube sections were quenched in a current of nitrogen gas to 100° F. (40° C.). In the third type, the sections were heated in a vacuum for one hour at 1200±25° F. (650±5° C.). The temperature was then ramped to 1850±25° F. (1010±5° C.) and held at that temperature for 30 minutes. Next, the tubes were quenched with nitrogen gas to 100° F. (40° C.). In the fourth type, the sections were heated in a vacuum for one hour at 1200±25° F. (650±5° C.). The temperature was then ramped to 1925±25° F. (1050±5° C.) and held at that temperature for 30 minutes. Next, the tube sections were quenched with nitrogen gas to 100° F. (40° C.). In the fifth type, the sections were heated in a vacuum for one hour at 1200±25° F. (650±5° C.). The temperature was then ramped to 2025±25° F. (1110±5° C.) and held at that temperature for 30 minutes. These tubes were then quenched with nitrogen gas to 100° F. (40° C.). This process produced five sets of tubes, each with a different annealing condition. Samples 30 mm in length were cut with side cutters from each set of the annealed tubes. Additionally, 30 mm long samples were cut from the original non-annealed tubing.

The malleability of the five tube types was then measured. FIG. 21 is a front view perspective of a holding fixture that was used for measuring the malleability of the tubes. FIG. 22 illustrates the holding fixture in end view perspective.

The holding fixture comprised an aluminum block 3 having a machined channel 2 cut in the top surface of the fixture. The channel had a semicircular cross-section and a diameter of 2.3 mm (0.092 inch). The fixture also comprised a V-shaped depression 5. The depression 5 was dimensioned such that it made an angle α of 30° and had a length λ of 10.5 mm as illustrated in FIG. 21. A tube 1 to be tested was placed in the channel 2 so that it spanned across the depression 5 as illustrated in FIGS. 21 and 22. A moveable probe 4 in the form of a cylindrical steel gauge pin having a diameter of 2.57 mm (0.101 inch) and a length of 5 cm (2 inches) was positioned directly above the tube 1 at the center of the depression 5. The probe 4 was attached with a chuck to an instrument that measured force (not shown in the Figures). One suitable instrument is a Chatillon TDC Force Measurement System manufactured by Willrich Precision Instrument, Cresskill, N.J., fitted with a 110 Newton (25-pound force) gauge.

Measurement of malleability was initiated by first slowly lowering the probe 4 until it just made contact with the tube 1. The force readout on the force gauge was then set to zero. The probe 4 was then slowly lowered in increments of 0.02 to 0.05 mm. At each increment of travel, the instantaneous force was recorded. The initial force or load at each displacement was recorded quickly since force dissipated quickly as the malleable sample relaxed and bent incrementally under the applied force. This process was continued until the tube 1 was bent to the 30° angle defined by the sloping walls of the V-shaped depression 5. Displacement was continued beyond this point to complete the test. Instantaneous load as a function of displacement was then plotted. The inflection point on the load versus displacement curve represented the malleability of the sample. At least 4 measurements were made for each annealing condition. A set of non-annealed tubes were measured under the same conditions. The results of this set represented the experimental control for comparison purposes.

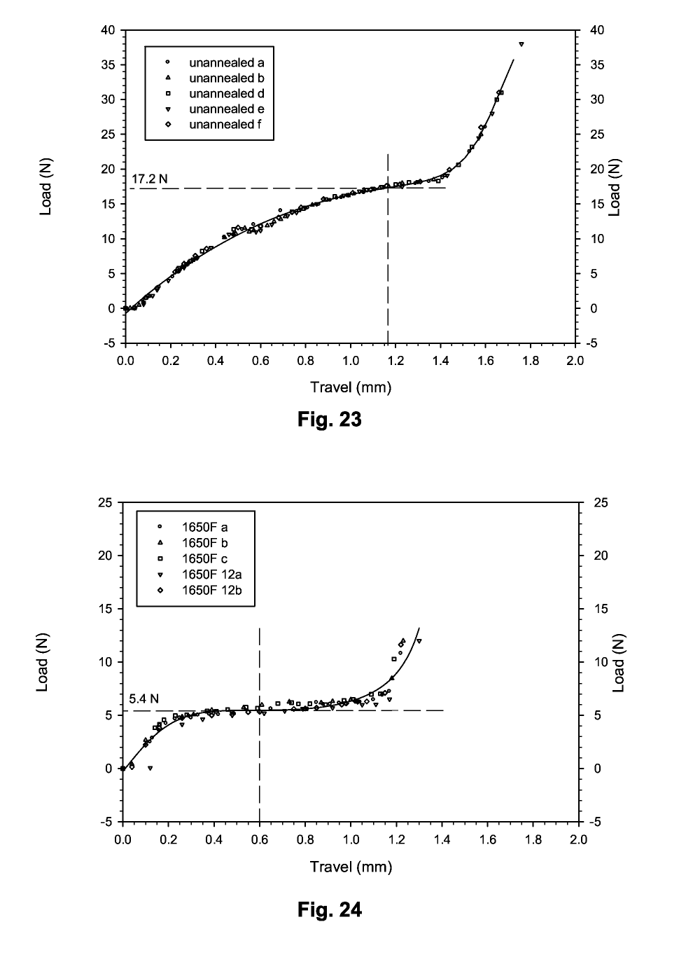

FIG. 23 shows the measured load vs. travel curve for the non-annealed tubes. The inflection point on the curve representing the malleability of the non-annealed grade 304 stainless steel tubes was 17.2 Newtons.

FIG. 24 illustrates the measured load vs. travel curve for the tubes annealed at 1650° F. (900° C.). The inflection point on the curve showed the malleability to be 5.4 Newtons.

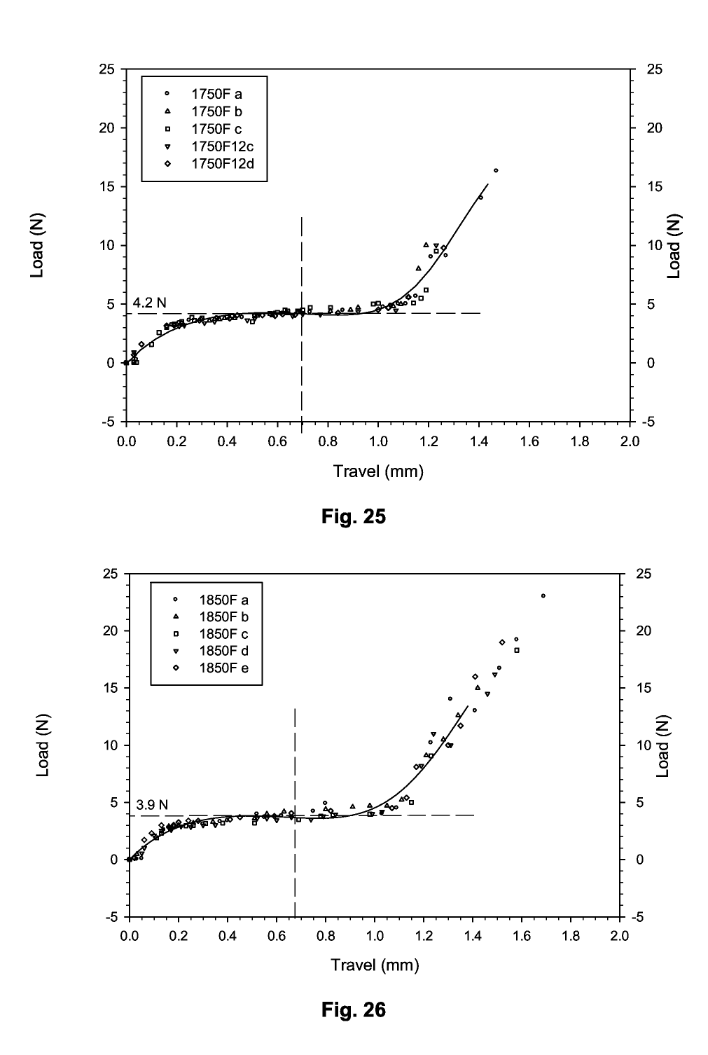

FIG. 25 illustrates the measured load vs. travel curve for the tubes annealed at 1750° F. (955° C.). The inflection point on the curve representing the malleability of the tubes is 4.2 Newtons.

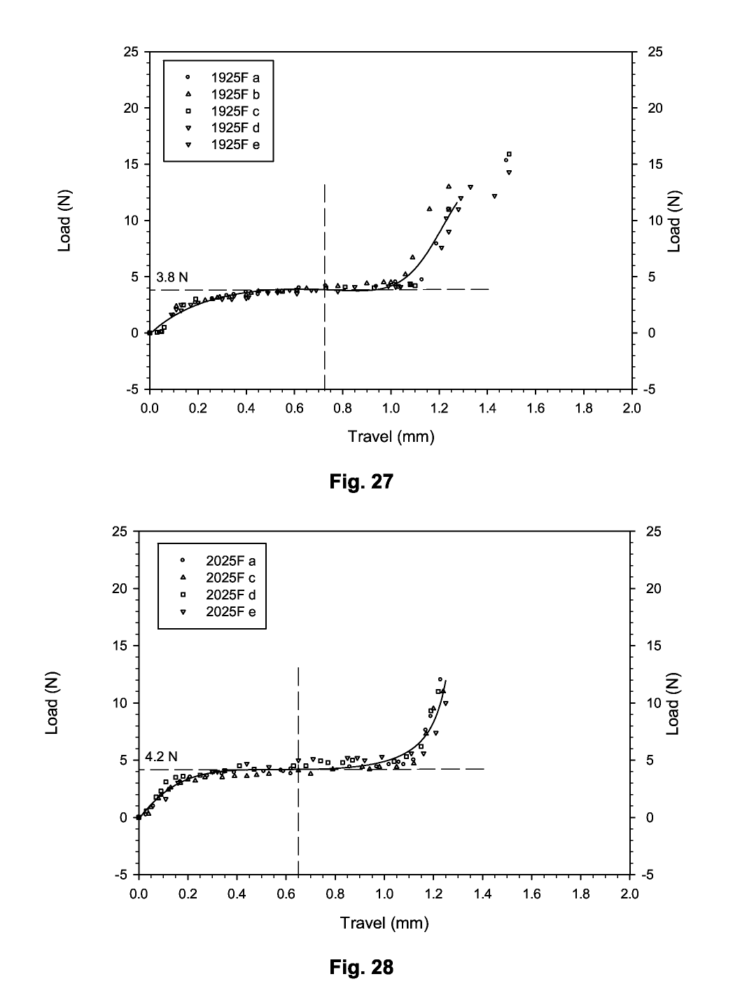

Likewise, FIGS. 26, 27 and 28 illustrate the measured load vs. travel curves for the tubes annealed at 1850° F. (1010° C.), 1925° F. (1050° C.), and 2025° F. (1110° C.), respectively. The inflection points on the curves representing the malleabilities of the annealed tubes are 3.8 Newtons, 3.8 Newtons and 4.2 Newtons, respectively.

### Example 2

### Fabrication of Dilators Having Differing Malleabilities

A series of frontal sinus recess dilators were fabricated with the annealed and non-annealed tube stock prepared in Example 1. First, osmotic engines were prepared. Polyethylene oxide was sifted through a 100-mesh sieve and 8.50 grams was collected in a 250 ml beaker. The polyethylene oxide had a molecular weight of 7 million and was supplied by Colorcon, Inc., West Point, Pa., as Polyox™ grade WSR 303 LEO, NF. Sodium chloride, USP grade, was ground in a mortar with a pestle and passed through a 100 mesh sieve. The sized sodium chloride (15.0 g) was added to the polyethylene oxide. Hydroxypropyl methylcellulose (1.25 g) was then passed through a 100 mesh sieve and added to the weighed powders. The hydroxypropyl methylcellulose was supplied by Dow Chemical Company, Midland, Mich., as grade Methocel™ E5 Premium LV. The three powders were next mixed with a spatula. Anhydrous ethyl alcohol (9 cm³), grade SDA 3A, was slowly added to the powders while stirring with a spatula until a uniformly damp mass was formed. The resulting damp mass was then passed with a spatula through a 40 mesh sieve to form damp pellets. The pellets were then air dried at room temperature overnight. The resulting dried composition was passed again through a 40 mesh sieve to form granules. The resulting granules were weighed and transferred to a 120 cm³ screw capped jar. Next, an amount of magnesium stearate (grade NF) as supplied by Spectrum Chemical Manufacturing Company, Gardena, Calif.) equal to 1% of this weight was passed through an 80 mesh sieve. The sized magnesium stearate was added to the dried granules and the composition was tumble mixed for one minute to form the osmotic engine granulation.

Next, portions of the granulation weighing a nominal amount of 23.8 mg were compressed at 270 Newtons (60 pounds force) with a Carver press fitted with core rod tooling. The tooling was biconvex with an outside diameter of 2.6 mm and an inside diameter 0.92 mm. The nominal height, measured from dome to dome, of the resulting osmotic tablets was 3.5 mm.

Flexible discs were injection molded from polyurethane (Tecophilic® grade HP-93A-100 supplied by Lubrizol Advanced Materials, Inc. Wilmington, Mass.). This grade of polyurethane has a Shore hardness of 83 A, a flex modulus of 200 atm (2,900 psi) as measured by ASTM method D790, and a percent ultimate elongation in the dry and hydrated states of 1040% and 620%, respectively, as measured by ASTM method D412. The flexible discs were of cylindrical geometry with a central hole. The outside diameter of the disc was 2.6 mm, the inside diameter was 0.91 mm, and the width was 1.6 mm. The discs had a nominal weight of 6.6 mg.

Dilator end caps were made as follows. Extruded polyurethane (Tecophilic® grade HP-93A-100) tubing having an outside diameter of 2.6 mm and inside diameter of 0.97 mm was cut into 2.0 mm lengths to form dilator end caps. The end caps had a nominal weight of 11.1 mg.

Next, two stainless steel (grade 316) frustum-shaped elements were machined on a lathe. The frustum elements had an overall length of 7 mm, a diameter of 3.2 mm at one end, and a diameter of 2.2 mm at the opposite end. A central lumen having a diameter of 0.66 mm (26 mils) was drilled through the frustum elements.

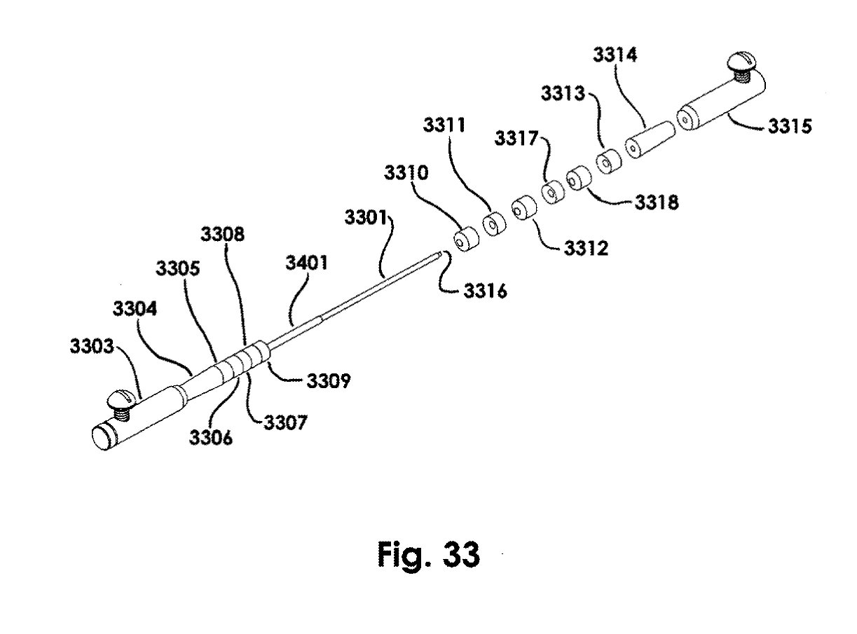

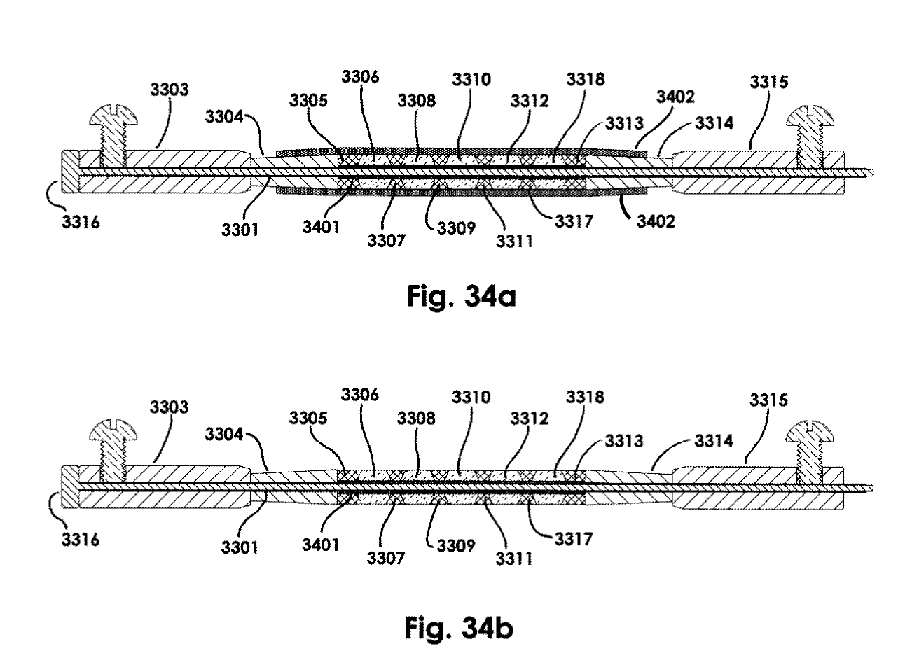

FIG. 33 shows a drawing of the assembly of a dilator. In FIG. 33, the stainless steel hypotube 3301 (grade 304) having an outside diameter of 0.64 mm (25 mils) and an inside diameter of 0.51 mm (20 mils) from Example 1 that was annealed at 1110° C. (2025° F.) was roll cut with a razor to a length of 77 mm. An 11 mil diameter by 2 inch long gauge pin 3316 was next inserted inside the cut length of hypotube 3301 to stiffen it for handling. A 20 mm length of poly ether ether ketone (PEEK) tubing 3303 having an outside diameter of 2.1 mm (82 mils) and an inside diameter of 0.81 mm (32 mils) was then cut. The PEEK tubing 3303 was slipped over one end of the hypotube 3301. This PEEK tubing 3303 served as a handle in later processing. One open end of the hypotube 3301 was flared to a diameter of 0.89 mm (35 mils) using a hardened steel, bullet-shaped tool with a pointed tip. The largest diameter of the flaring tool was 3.2 mm (125 mils). The tool had a Rockwell hardness of 63. The flaring tool was held in a chuck that rotated at high speed in a Servo drill press during the flaring process. Next, a frustum element 3304 as described above was threaded onto the hypotube such the narrow end of the frustum element abutted the PEEK handle. A 21.3 mm length of extruded polyurethane tubing (Tecophilic® grade HP-93A-100) with an outside diameter of 0.89 mm (35 mils) and an inside diameter of 0.61 mm (24 mils) was cut with a razor. The polyurethane tubing 3401 (see FIG. 34a) was wrapped in a damp paper towel and hydrated for four minutes to swell it. The swollen tube 3401 was then slipped onto the hypotube 3301 such that it abutted the wide end of frustum element 3304. This subassembly was then placed in a 50° C. forced air oven for 7 minutes to shrink the swollen tubing 3401 down onto the hypotube 3301. The resulting shrunken tubing serves as the inner membrane 3401 of the dilator.

Next, two end caps, four osmotic engines, and three flexible discs were threaded onto the inner membrane in the following sequence. First, a 2.0 mm end cap 3305 (see FIG. 33 and FIGS. 34a and 34b) as described above was threaded onto the inner membrane such that it abutted the frustum element 3304. Then, an osmotic tablet 3306 as described above was threaded over the inner membrane such that it abutted the end cap 3305. Next, a flexible disc 3307 as described above was threaded over the inner membrane such that the flexible disc 3307 abutted the osmotic tablet 3306. Then, a second osmotic tablet 3308 was threaded over the inner membrane such that it abutted the flexible disc 3307. Then, a second flexible disc 3309 was threaded over the inner membrane such that it abutted the second osmotic tablet 3308. Next, a third osmotic tablet 3310 was threaded over the inner membrane such that it abutted the flexible disc 3309. Then, a third flexible disc 3311 was threaded over the inner membrane such that it abutted the third osmotic tablet 3310. Next, a fourth osmotic tablet 3312 was threaded over the inner membrane such that it abutted the third flexible disc 3311. Then, a fourth flexible disc 3317 was threaded over the inner membrane such that it abutted the fourth osmotic tablet 3312. Next, a fifth osmotic tablet 3318 was threaded over the inner membrane such that it abutted the fourth flexible disc 3317. Finally, a second 2.0 mm end cap 3313 was threaded over the inner membrane such that it abutted the fifth osmotic tablet 3318. In other embodiments, additional (or fewer) osmotic tablets and flexible discs may be included in the dilator as desired.

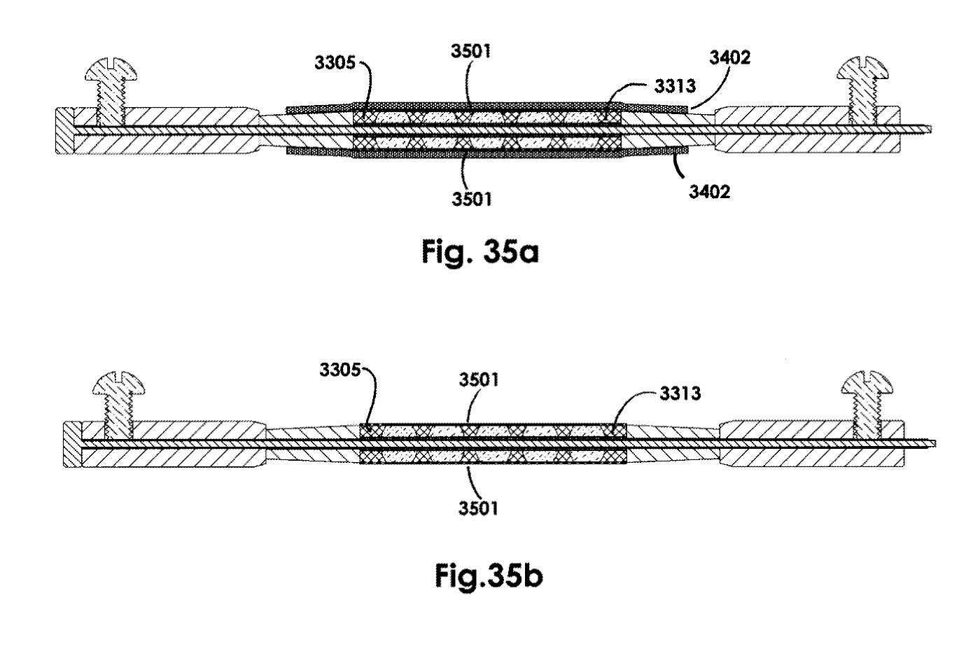

In some instances, a section of extruded tubing 3501 (see FIGS. 35a and 35b was disposed over the threaded components of the dilator to serve as the outer membrane of the dilator. In these cases, the section of extruded tubing 3501 was cut to length. The composition of the tubing was 90 wt % polyurethane (Tecophilic® grade HP-9A-100) and 10 wt % polyvinyl pyrrolidone (Kollidon® 12 PF sold by the BASF Corporation, Florham Park, N.J.). The outside diameter of the tubing was 3.33 mm (131 mils) and the inside diameter was 2.72 mm (107 mils). A 21.4 mm length of this tubing was cut. The cut length of tubing 3501 was slipped over the threaded components of the dilator such that it spanned from end cap 3305 to end cap 3313.

Next, a second frustum element 3314 (see FIGS. 33, 34a and 34b) was slipped onto the hypotube 3301 such that the wide end of the frustum element 3314 abutted the second end cap 3313. Then, a 22.2 mm length of PEEK tubing 3315 having an outside diameter of 2.1 mm (82 mils) and an inside diameter of 0.81 mm (32 mils) was then cut. The resulting PEEK tubing 3315 was slipped onto the hypotube 3301 such that it abutted the second frustum element 3314. Then, the hypotube was flared to an outside diameter of 0.89 mm (35 mils) such that the sequence of end caps, flexible discs, and osmotic tablets were held snugly together.

Referring to FIGS. 34a and 35a, FEP fluoropolymer heat shrink tubing 3402 (sold by Zeus, Inc., Orangeburg, S.C.) with an outside diameter of 4.0 mm (158 mils) and an inside diameter of 3.6 mm (141 mils) was cut to a length of 39 mm. The resulting tube 3402 was slipped over the end caps, flexible discs and osmotic tablets such that it had a 7 mm overlap onto the frustum elements at each end.

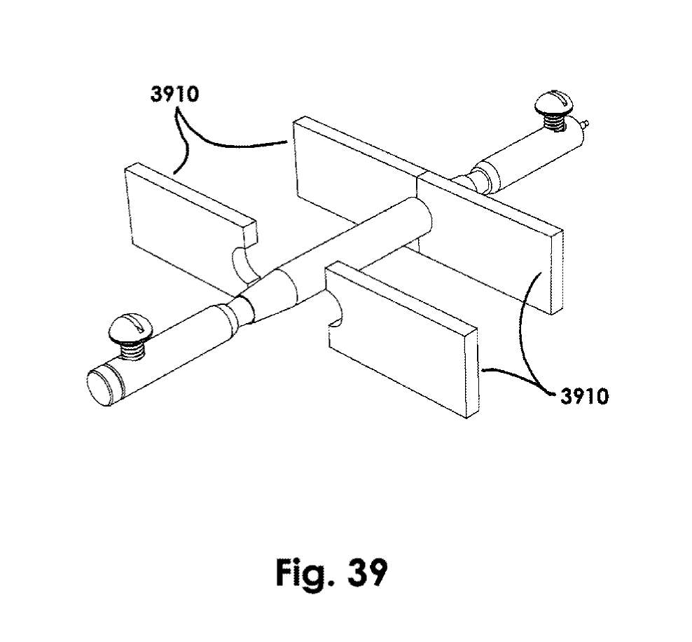

Next, the heat shrink tubing 3402 was shrunk by holding the PEEK handles (3303 and 3315) and rolling the assembly over a metal block heated to 200° C. (390° F.). This process produced a closed chamber bounded by the frustum elements (3304 and 3314) at each end and the inner surface of the wall of the heat shrink tubing 3402. Then, the components of the device were thermally bonded using a model 220-B Beahm thermal welder with split dies 3910 (see FIG. 39). Diameter of the opening in the die was 3.3 mm (130 mils) and the width of the die was 1.0 mm. The die 3910 was heated to 200° C. (390° F.). One 30 second heat application was made over each disc and two 30 second applications were made over each end cap. The resulting process melted and bonded the outer membrane 3501 (see FIGS. 35a and 35b) to the end caps and to the discs and the inner membrane 3401 to the end caps and to the discs. A small slit was made with a razor on the edge of the heat shrink tubing and it was then peeled off in a spiral pattern and removed. Thus, four chambers were formed in the process where each chamber was occupied by an osmotic engine.

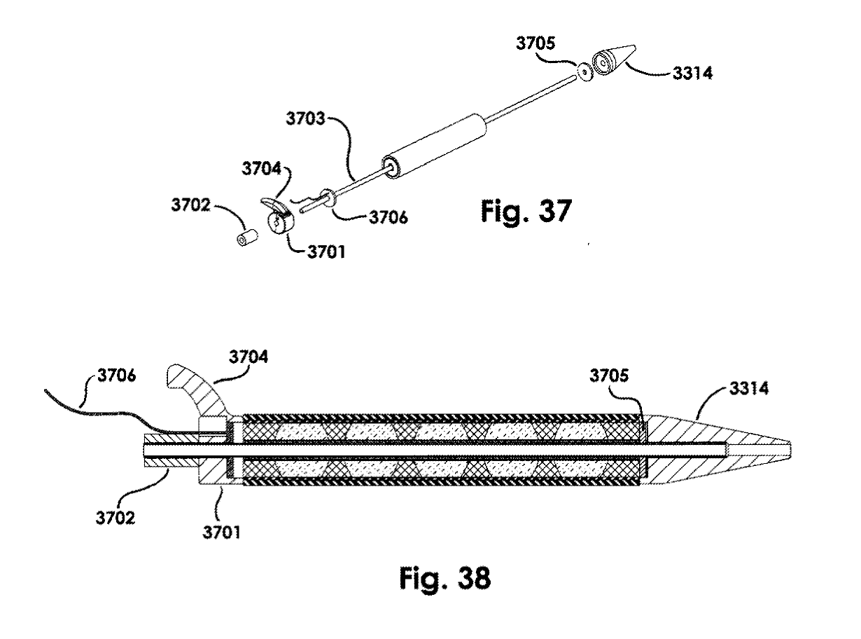

Next, the exposed outer membrane of the dilator was wrapped in a temporary layer of Parafilm® M to protect the membrane from exposure to metal dust filings. One end of the PEEK handle was ground with a belt sander to grind down the flared end of the hypotube. The PEEK handle 3303 and frustum element 3304 were then removed. A small drop of medical grade cyanoacrylate cement (Loctite® 4011 sold by Henkel Corporation) was placed on the exposed hypotube adjacent to the end cap. Then, a polymeric anchor 3701 was slipped onto the hypotube such that it was in contact with the end cap (see FIG. 37 and FIG. 38). As shown in FIGS. 37 and 38, the polymeric anchor included one petal 3704. In other embodiments, the polymeric anchor may include two or more petals. The anchor was injection molded from Pebax® polyether block amides and had a central hole having a diameter of 0.69 mm (27 mils). The petal 3704 extended outward from the center of the central hole by 3.94 mm (155 mils). The petals were designed to prevent the dilator from being pushed too far into the sinus cavity when the dilator is placed in a stenotic recess of a frontal sinus.

Next, referring to FIG. 37, a section of PEEK tubing having an outside diameter of 1.6 mm (64 mils) and an inside diameter of 0.64 mm (25 mils) was cut to a length of 1.8 mm (70 mils) to form a sleeve 3702 (see FIGS. 37 and 38). A drop of adhesive was placed on the hypotube such that it was also in contact with the anchor 3701. The sleeve 3702 was then slipped onto the hypotube 3703 such that it abutted the anchor 3701. The exposed hypotube was roll cut with a razor leaving a 1 mm (40 mils) length of hypotube protruding. The protruding hypotube was flared with the flaring tool described earlier. Then the flared end was ground with a belt sander until the end of hypotube was nearly flush with the PEEK sleeve.

Next, referring back to FIGS. 34a and 34b, the distal end of the device was ground with a belt sander to remove the flair on the distal end of the dilator. The PEEK handle 3315 and frustum element 3314 were removed. A small drop of adhesive was placed on the resulting exposed hypotube adjacent to the end cap 3313 and the frustum element 3314 was replaced such that it abutted the end cap 3313 and was cemented to the end cap 3313. In some instances, a distal stop 3705 was placed between the end cap 3313 and the frustum element 3314 (see FIGS. 37 and 38). In some instances, a proximal stop 3706 was placed between end cap 3305 and the frustum element 3304. In some instances, the proximal stop 3706 included a tether, which facilitated removal of the dilator from the subject. The exposed hypotube was roll cut with a razor exposing a 1 mm (40 mils) length of hypotube. The end of the hypotube was flared with the flaring tool and ground with a belt sander such that it was nearly flush with the frustum element 3314. The Parafilm M was removed to complete fabrication of the dilator.

Next, additional dilators were fabricated using the same process as described above for the dilator fabricated with hypotubes annealed a the 1110° C. (2025° F.) condition but from the hypotubes annealed at the various other conditions described in Example 1. Dilators were therefore prepared with non-annealed hypotubes, and hypotubes annealed at 900° C., 950° C., 1010° C., and 1050° C. (i.e., 1650° F., 1750° F., 1850° F., and 1925° F.).

### Example 3

### Placement of Dilators Having Differing Malleabilities in Cadaver Frontal Sinus Recesses

The dilators fabricated in Example 2 were used to assess ease of dilator placement into the frontal sinus recess of a cadaver. The dilators were mounted on a cannula having a slot at the end. The proximal end of the dilator was mounted in the slot. Under endoscopic guidance, the mounted dilator and cannula were then threaded through a nostril into the nasal cavity and advanced to the frontal sinus recess. Ease of placement of the dilator into the frontal sinus recess was then assessed by a Board Certified otolaryngologist, head and neck surgeon. The dilator fabricated with the non-annealed hypotube (hypotube malleability of 17.2 Newtons) was judged to be too stiff to maneuver into the frontal sinus recess. The dilator fabricated with the hypotube annealed at 1650° F. (hypotube malleability of 5.4 Newtons) was also judged to be too stiff to maneuver into the frontal sinus recess. The dilators having hypotubes annealed at 1750° F., 1850° F., and 1925° F., with hypotube malleabilities of 4.2 Newtons, 3.9 Newtons and 3.8 Newtons, respectively, were judged to be sufficiently malleable to allow placement within the frontal sinus recess. The dilators having hypotubes annealed at 2025° F. (hypotube malleability of 4.2 Newtons), were judged to be slightly too malleable for optimal maneuvering into the frontal sinus recess.

### Example 4

### Fabrication of Malleable Dilator with Double-Layer Outer Semipermeable Elastic Membrane

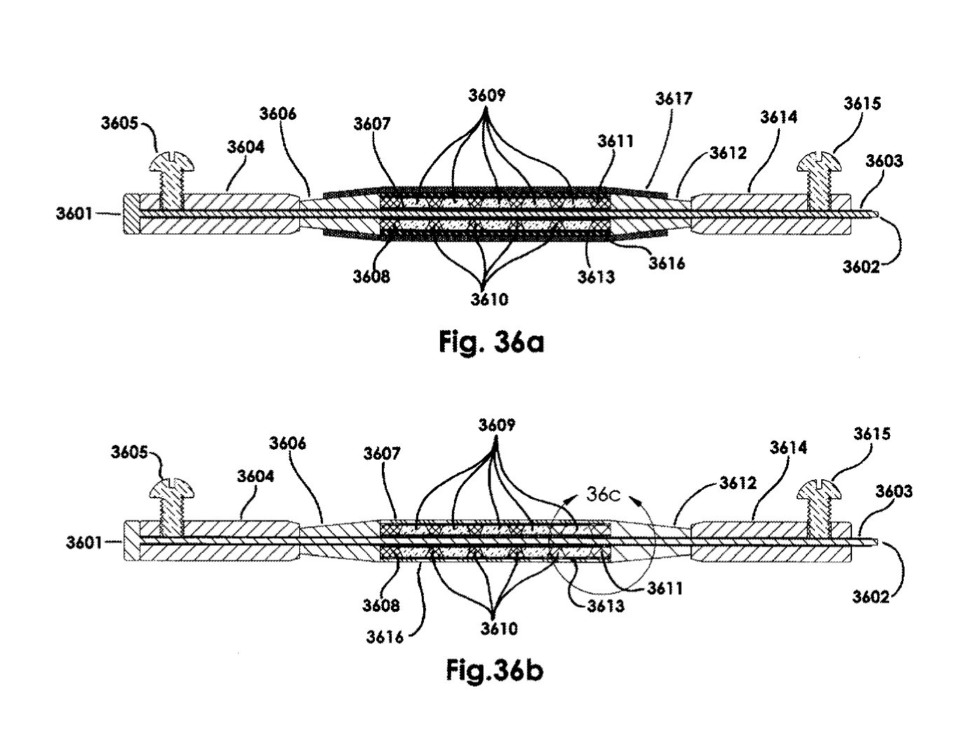

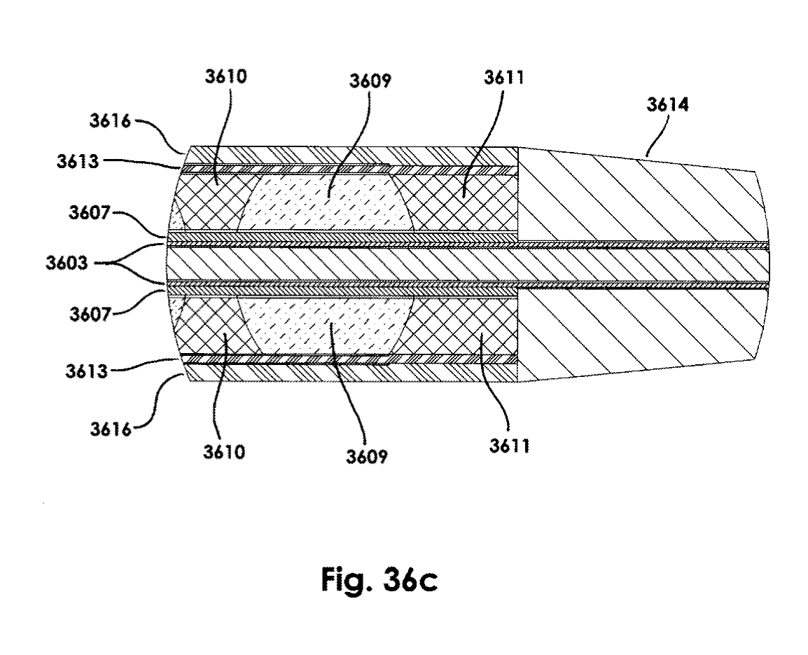

A dilator with a double-layer outer membrane was fabricated (see FIGS. 36a, 36b and 36c). First, a stainless steel cylinder 3601 having a diameter of 3.2 mm (125 mils) and a thickness of 1.9 mm (75 mils) was welded to the end of a stainless steel wire 3602 having a length of 68 mm and diameter of 0.43 mm (17 mils). The end of the wire was welded to the round face of the cylinder and was centered in the face. The wire was positioned normal to the face of the cylinder. The wire served as a processing aid to stiffen malleable hypotubes during handling and fabrication.

The stiffening wire 3602 was inserted into the lumen of a stainless steel (grade 304) hypotube 3603 having a length of 30.5 cm (1 ft). The hypotube had an inside diameter of 0.5 mm (20 mils) and an outside diameter of 0.6 mm (25 mils) and had been annealed at 955° C. (1750° F.). With the stiffening wire in place, a 90 mm length of the hypotube was roll cut with a razor.

A handle 3604 fabricated of PEEK having a length of 2.5 cm (1.0 in), and an inside diameter of 0.7 mm (27 mils), and an outside diameter of 3.2 mm (125 mils), and a set screw 3605 at one end was slipped onto the hypotube such that the end having the set screw abutted the cylinder 3601 of the stiffening wire. A set screw 3605 in the handle was tightened gently. The removable handle was used for holding the proximal end of the dilator during the fabrication process.

The exposed cut end of the hypotube was de-burred with a metal file. Next, a stainless steel frustum element 3606 having a diameter of 2.6 mm (102 mils) at the larger end, an outside diameter of 2 mm (80 mils) at the smaller end, an overall length of 6.2 mm (245 mils) and a central hole having a diameter of 0.7 mm (26 mils) was slipped onto the hypotube 3603 such that the narrow end of the frustum element 3606 abutted the PEEK handle 3604.

A 20.5 mm length of polyurethane tubing 3607 (Tecophilic® HP-93A-100) was cut with a razor. The tubing 3607 had been extruded to an inside diameter of 0.6 mm (24 mils) and an outside diameter of 0.9 mm (35 mils). The resulting length of tubing was wrapped in a paper towel dampened with water and hydrated for 3 minutes. The swollen membrane 3607 was then slipped onto the hypotube 3603 such that it abutted the wide end of the frustum element 3606. While air drying, the swollen tube was then massaged manually down to the original 20.5 mm length. Next, the assembly was placed in a 50° C. forced air oven to shrink the polyurethane tubing 3607 which formed the inner membrane 3607 of the dilator.

Next, polyurethane end caps were fabricated. 2.0 mm sections were cut with a razor from polyurethane tubing (Tecophilic® HP-93A-100) that had been extruded to an outside diameter of 2.6 mm (104 mils) and inside diameter of 1 mm (38 mils). Flexible discs were fabricated by cutting 1.4 mm sections from the same polyurethane tube stock.

One end cap 3608 was threaded over the inner membrane 3607 such that it abutted the frustum element 3606. Then, five osmotic tablets 3609 and four flexible discs 3610 were threaded over the inner membrane 3603 such that a disc 3610 was positioned between each adjacent tablet 3609 and the 9-part stack abutted the end cap 3608. The composition and fabrication process used to make the osmotic granulation and tablet compress were equivalent to those described in Example 2 except that the nominal dome-to-dome dimension of the tablets was 2.2 mm and the nominal weight of the tablets was 14.4 mg. The outside diameter of the osmotic tablets was 2.6 mm and the inside diameter was 0.97 mm. A second end cap 3611 was threaded onto the inner membrane 3603 such that it abutted the last-placed (i.e., distal) osmotic tablet 3609 which served as the end cap on the distal end of the dilator.

Then, a second frustum element 3612 having the same dimensions as the first frustum element 3606 was slipped onto the hypotube 3603 such that it abutted the distal end cap 3611.

Next a 30 mm length of heat shrink tubing was cut with a razor. Composition and dimensions of this tubing were equivalent to those described in Example 2. The heat shrink tube was slipped over the assembly such that it overlapped both end caps (3608 and 3611). A second PEEK handle 3614 having a length of 2.5 cm (1 in), an inside diameter of 0.7 mm (27 mils), and an outside diameter of 3.3 mm (125 mils), and a set screw 3615 at one end was slipped onto the hypotube 3603 such that it abutted the second frustum element 3612. The set screw of the handle was positioned nearest the exposed cut end of the hypotube. Then, the set screw was gently tightened to secure the handle to the assembly.

With the stiffening wire in place and the heat shrink tubing overlapping both frustum elements (3606 and 3612), the heat shrink tubing was shrunk by holding both handles of the assembly and rotating the assembly over the heating block of a Beahm model 220-B thermal welder set at 390° F. (200° C.). The assembly was then cooled to room temperature.

Next, the end caps and flexible discs were bonded to the inner membrane. With the heat shrink tubing in place, end caps of the assembly were thermally bonded using a 1.0 mm wide split die 3910 (see FIG. 39) having an inside diameter of 3.8 mm (150 mils) for 90 seconds at 200° C. Pressure of the die closure was set at 4 atm (60 psi). The 1.0 mm die 3910 was centered in the middle of the 2.0 mm end caps during bonding. The assembly was then cooled to room temperature. Next, the flexible discs were bonded to the inner membrane. Each disc was bonded at 390° F. (200° C.) for duration of 65 seconds. The 1.0 mm wide die 3910 was centered over each disc. The assembly was then cooled to room temperature. Then, a small slit was made on the edge of the heat shrink tubing and this tubing was then peeled off and discarded.

Next, the end caps and flexible discs were bonded to a first outer membrane 3613. The span from end cap to end cap was measured to be 20.6 mm. A 20.6 mm length of extruded polyurethane tubing (Tecophilic® HP-93A-100) was cut with a razor. The outside diameter of the tubing was 3.1 mm (123 mils) and inside diameter was 2.8 mm (110 mils). The cut length of tubing 3613 was slipped onto the assembly such that is spanned end cap to end cap. A 30 mm length of heat shrink tubing equivalent to what was used above was slipped over the assembly such it was centered over the assembly and overlapped both frustum elements (3606 and 3612). With the stiffening wire still in place, the heat shrink tubing was rolled over the heating block of the Beahm model 220-B thermal welder set at 390° F. (200° C.) to shrink the tubing. The assembly was then cooled to room temperature. Next, the assembly was thermally bonded with a 20.9 mm wide split die having an inside diameter of 4 mm (160 mils) for 30 seconds at 390° F. (200° C.). Pressure of the die closure was set at 4 atm (60 psi). The assembly was cooled to room temperature. The edge of the heat shrink tubing was cut and this tubing was peeled off in a spiral pattern and discarded. Flash of up to 1 mm that had formed on the frustum elements during the bonding cycle was trimmed off. At this point in the fabrication process, each of the five osmotic engines was encapsulated within the polyurethane membrane.

Next, the second outer membrane 3616 was bonded to the assembly. The composition of the second outer membrane was 90 wt % polyurethane (Tecophilic® HP-93A-100) and 10 wt % polyvinyl pyrrolidone (Kollidon® 12 PF). This composition had been extruded to tubing with an outside diameter of 3.3 mm (131 mils) and inside diameter of 2.7 mm (107 mils). The second outer membrane tube 3616 stock was cut with a razor to a length of 17.8 mm. Then, the cut length of second outer membrane 3616 was immersed in 50 cm³ of dry acetone (reagent grade A.C.S. with a moisture content of less than 0.5 wt %) and solvated and swollen for 20 minutes at room temperature. Next, the set screw 3615 of the distal PEEK handle 3614 was loosened and the distal handle was removed. The swollen outer membrane 3616 was slipped over the stack of engines such that it overlapped both frustum elements equally on both sides. The distal PEEK handle 3614 was replaced and the set screw was tightened. Next, the dilator was placed in a 50° C. force air oven for 21 hours to dry off the acetone. The outer membrane had shrunk to a length such that it overlapped each frustum element by 0.3 mm. The 0.3 mm overlap was trimmed off with a razor.

Next, the set screw of the PEEK handle 3615 on the distal side was loosened and the handle was removed. The frustum element 3612 on the distal end of the assembly was removed. It was next replaced with a larger frustum element. The larger frustum element had a diameter on the wide end of 3.3 mm (131 mils), a diameter on the narrow end of 2 mm (80 mils) and a length of 7.6 mm (300 mils). The larger frustum element had a central hole spanning the length with a diameter of 0.7 mm (26 mils). The larger frustum element was threaded onto the hypotube such that it abutted the distal end cap 3611. The distal PEEK handle 3614 was replaced such that it abutted this larger frustum element and the set screw was tightened lightly.

Next, the handle 3604 and frustum element 3606 on the proximal end of the assembly were removed. Then, a 30 mm length of heat shrink tubing equivalent to what was used earlier in this example was cut. The cut length of heat shrink tubing was slipped over the engines such that it overlapped the end caps by the same amount at each end. Next, a larger frustum element of equivalent dimensions to the one placed on the distal end of the assembly was threaded onto the hypotube such that it abutted the proximal end cap. The stiffening wire and PEEK handle were replaced and the set screw was tightened.

Next, the heat shrink tubing was positioned such that it overlapped the frustum elements by the same amount at each end. Then, the heat shrink tubing was shrunk by rolling it over the heating block of a Beahm model 220-B thermal welder set at 390° F. (200° C.). The assembly was cooled to room temperature. Then, the second outer membrane was thermally bonded in only two locations to the first outer membrane using a 1.0 mm wide split die with an opening of 3.8 mm (150 mils) for 115 seconds at 390° F. (200° C.). Bonding was performed over the two end caps. In this process, the 1.0 mm wide die was centered over the 2.0 mm wide end caps. The dilator was then cooled to room temperature.

A small cut was made with a razor on an end of the heat shrink tubing. The heat shrink tubing was then peeled off in a spiral pattern and discarded. Approximately 0.5 mm of flash from thermal bond bonding of the end caps was present on both frustum elements. This flash was trimmed off with a razor.

The set screw of the proximal PEEK handle was loosened and the handle, stiffening wire and frustum element were removed. The exposed hypotube was roll cut with a razor. Location of the cut was adjacent to the end cap such that approximately 4 mm (160 mils) of hypotube were exposed. Then, an anchor with two petals and PEEK sleeve were attached to the hypotube with adhesive as described in Example 2. The exposed end of the hypotube was then flared using the process as described in Example 2.

Next, the set screw of the distal PEEK handle was loosened and the handle was removed. The hypotube was cut adjacent to the frustum element such that 1 mm (40 mils) of hypotube were left exposed. A small drop of Loctite® 4011 adhesive was applied to bond the hypotube and frustum element. The cut end of the hypotube was then flared using the process described in Example 2.

Average widths of the flexible discs of this dilator after thermal bond were measured using a Mitotoyo TM microscope. Average width of the four flexible discs was 1.3 mm.

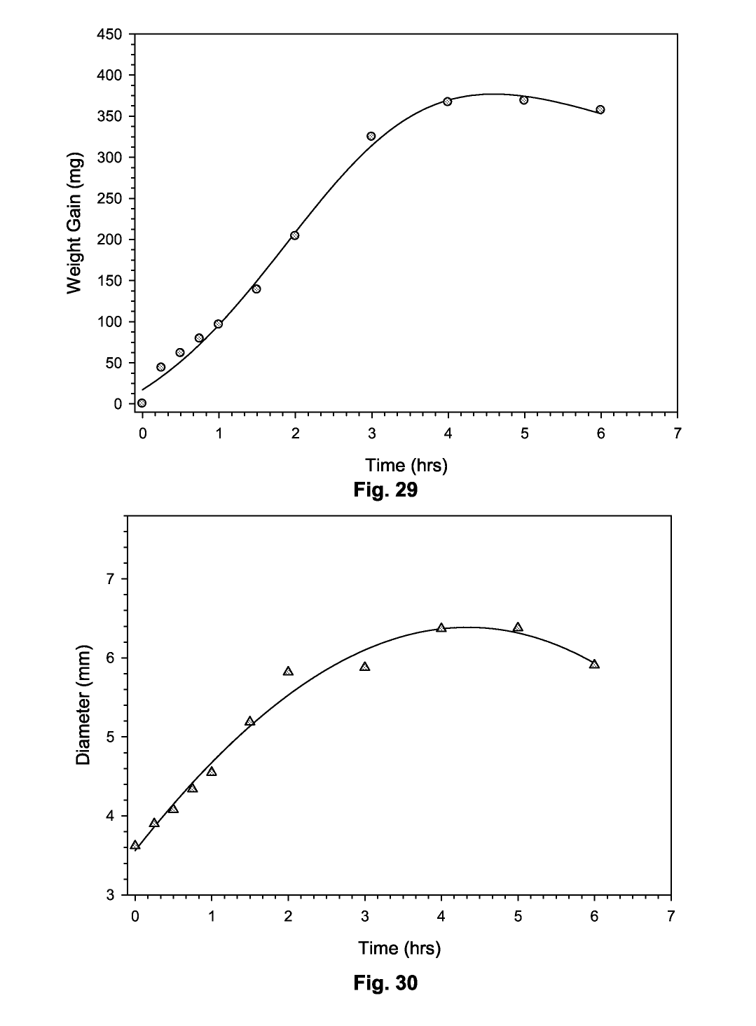

The dilator was tested for expansion over a duration of 24 hours with a USP type 2 paddle tester using 500 ml of de-ionized 37° C. water stirred at 50 rpm. The dilator swelled and retained the osmotic hydrogel within the device for the full duration of the test. Weight of the dilator was monitored with an analytical balance and diameter of the dilator versus time was measured with calipers. The swelling profile of weight versus time is illustrated in FIG. 29. The corresponding plot of diameter versus time is illustrated in FIG. 30.

The dilator expanded to a diameter of 4.6 mm in 60 minutes and to 5.4 mm in 90 minutes. The contraction cycle commenced between 4 and 5 hours.

### Example 5

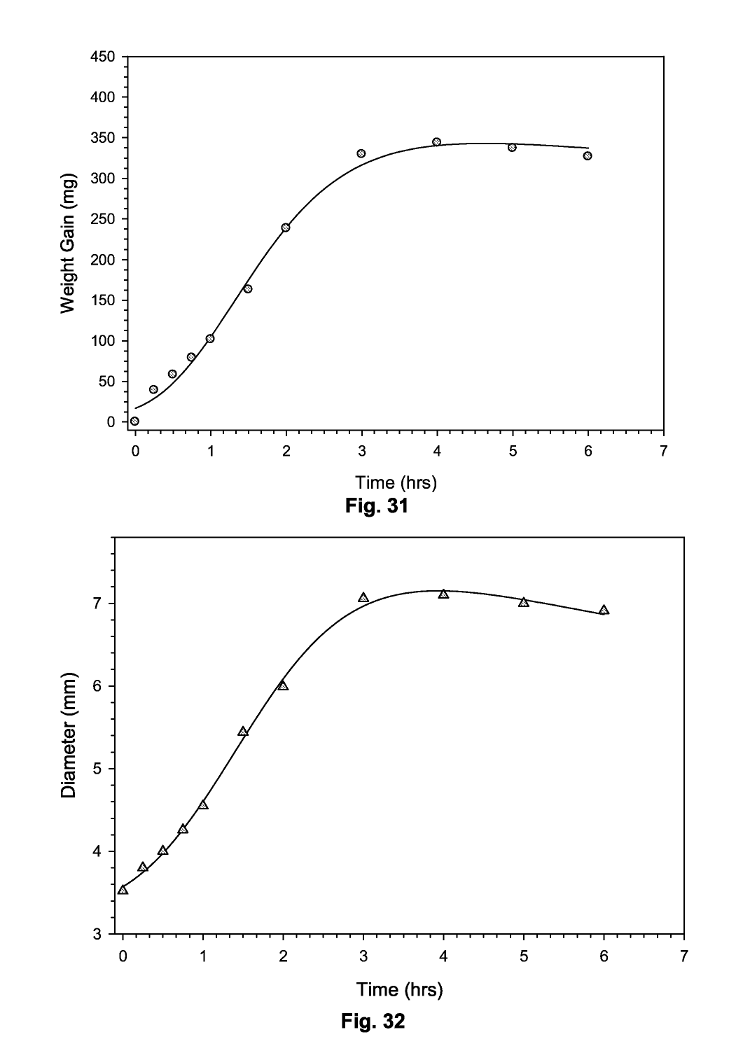

Another malleable dilator with a double-layer outer semipermeable elastic membrane was made equivalent to the dilator of Example 4 except the nominal width of the osmotic tablets was 2.5 mm and the nominal width of the flexible discs was 0.9 mm. The dilator swelled and retained the osmotic hydrogel within the device for the full duration of the test. The swelling profile of weight versus time is illustrated in FIG. 31. The corresponding plot of diameter versus time is illustrated in FIG. 32. The dilator expanded to a diameter of 4.6 mm in 60 minutes and to 5.4 mm in 90 minutes. The contraction cycle commenced between 4 and 5 hours.

### Example 6

Another batch of stainless steel (grade 304) hypotubes having a nominal inside diameter of 0.5 mm (0.020 in) and an outside diameter of 0.6 mm (0.025 inch) were annealed as described in Example 1 except the cut sections of tubes were annealed by heating in a vacuum for 45 minutes at 1600±25° F. (870±5° C.). The temperature was then ramped to 1750±25° F. (955±5° C.) and held at that temperature for 30 minutes. Finally, the tubes were quenched in a current of nitrogen gas to a temperature of 100° F. (40° C.).

### Example 7

The malleability of a dilator was measured. FIG. 40 is a front view perspective of a holding fixture that was used for measuring the malleability of the dilator. FIG. 41 illustrates the holding fixture in end view perspective.

The holding fixture comprised an aluminum block 3 having a machined channel 7 cut in the top surface of the fixture. The channel had a semicircular cross-section and a diameter of 2.3 mm (0.092 inch). The fixture also comprised a V-shaped depression 5. The depression 5 was dimensioned such that it made an angle α of 30° and had a length λ of 10.5 mm as illustrated in FIG. 40. A dilator 8 to be tested was placed in the channel 7 so that it spanned across the depression 5 as illustrated in FIGS. 40 and 41. A moveable probe 6 in the form of a cylindrical steel gauge pin having a diameter of 4.93 mm and a probe face 9 with a semicircular cross section with a diameter of 3.4 mm was positioned directly above the dilator 8 at the center of the depression 5. The probe 6 was attached with a chuck to an instrument that measured force (not shown in the Figures). One suitable instrument is a Chatillon TDC Force Measurement System manufactured by Willrich Precision Instrument, Cresskill, N.J., fitted with a 110 Newton (25-pound force) gauge.

Measurement of malleability was initiated by first slowly lowering the probe 6 until it just made contact with the dilator 8. The force readout on the force gauge was then set to zero. The probe 6 was then slowly lowered in increments of 0.02 to 0.05 mm. At each increment of travel, the instantaneous force was recorded. The initial force or load at each displacement was recorded quickly since force dissipated quickly as the malleable sample relaxed and bent incrementally under the applied force. This process was continued until the dilator 8 was bent to a 30° angle. Displacement was continued beyond this point to complete the test. Instantaneous load as a function of displacement was then plotted. The point on the load versus displacement curve where the dilator conformed to a 30 degree angle represented the malleability of the dilator.

FIG. 42 shows a graph of the measured load (N) vs. displacement (mm) for a dilator. The dilator included an axial member (i.e., a 304 stainless steel tube with an outside diameter of 0.6 mm and an inside diameter of 0.5 mm) annealed at 955° C. (1750° F.). The inflection point on the curve representing the malleability of the dilator was 23.1 Newtons, which corresponded to a probe displacement of 1.87 mm when the dilator was bent to 30 degrees.

### Example 8

An osmotic engine composition comprising an osmotic agent and a swellable polymer was prepared as follows. First, 13.0 grams of sodium chloride, USP grade, was weighed and dried in 50° C. forced air for four hours. The dried osmoagent was then ground with a pestle in a mortar and passed through a sieve having 100 wires per inch. 12.5 grams of the resulting dried and sized sodium chloride was weighed and transferred to a 250 cm³ beaker. Next, 11.0 grams of swellable and fibrous polymer, USP/NF grade, low-substituted hydroxypropyl cellulose (LH-11 sold by Shin-Etsu Chemical Company, Tokyo, Japan), was weighed and passed through a sieve having 100 wires per inch. The LH-11 had a nominal particle size of 50 microns and a nominal hydropropoxyl content of 11 weight percent with a molar substitution of about 0.2-0.4. The sized and weighed LH-11 was added to the sodium chloride. 1.3 grams of hydroxypropyl cellulose (HPC) that had been previously sized using 100 mesh was weighed. The hydroxypropyl cellulose had a molecular weight of about 80,000 grams per mole, a molar substitution of about 3.0 (Klucel™ EF sold by Ashland Specialty Ingredients, Wilmington, Del. The sized and weighed HPC was added to the weighed powders. The resulting composition was stirred with a spatula for 10 minutes to form a uniform dry blend. Next, 7 cm³ of anhydrous ethyl alcohol grade SDA-3A was slowly added with stirring to the mixture to form a uniform damp mass, which was then forced with a spatula through a sieve having 40 wires per inch onto a stainless steel pan. The resulting granules were then dried in a fume hood at room temperature overnight. Next, the dried granules were passed with a spatula through a sieve having 40 wires per inch and weighed. The weight was 22.5 grams. The weighed granules were placed in a 120 cm³ screw-capped jar. Next, 227 mg of tableting lubricant, magnesium stearate USP/NF grade, was weighed, passed through a mesh having 80 wires per inch, and then added to the granules. The blend was then tumbled for 1 minute to form the osmotic engine granulation.

The mechanical properties of the resulting granulation in compressed tablet form were then characterized. Aliquots weighing 125 mg of the granules were compacted manually on a Carver press fitted with 0.25 inch (0.64 cm) diameter round standard biconcave tablet tooling with a nominal compression force of 420 pounds force (1870 Newtons). The hardness of six tablets was measured on a hardness tester, model VK200 sold by Varian, Inc. The average hardness was 6.1 kiloponds. Friability on a sample of ten tablets was measured on friabilator, model FT-400 (Key International, Cranbury, N.J.) using 400 drops. Friability of the compacts was 0.2 percent.

### Example 9

Osmotic dilators having a double layered outer membrane comprising an extruded membrane layer and a dip coated membrane layer were next fabricated using the granulation prepared in Example 8. Aliquots of the osmotic engine granulation were weighed and filled into the cavity of round core rod tablet tooling. Within the round die cavity and at the bottom of the cavity was positioned a round lower punch having a face with standard concave configuration. The outside diameter of the lower punch was substantially similar to the inside diameter of the die cavity such that when powders were filled into the cavity, they were retained within the cavity. Attached to the lower punch was a small diameter round pin. The pin was positioned in the center of the lower punch and oriented vertically. The core rod tooling additionally included a round upper punch having a face with standard concave configuration. The outside diameter of the round upper punch was substantially equivalent to the inside diameter of the die cavity. The upper punch additionally had a bore. The inside diameter of the bore was substantially equivalent to the outside diameter of the pin. Tablets compressed with this tooling therefore were produced in the configuration of cylinders which cylindrical tablets had a central lumen where the lumen was parallel to the axis of compression. Additionally, the ends of the cylinders were rounded as formed by compression in the concave configuration of the tablet face tooling.

Aliquots of granulation from Example 8 at a nominal weight of 7.3 mg were weighed into the die cavity. The inside diameter of the cavity was 1.8 mm (0.071 inch) and the outside diameter of the center pin was 0.685 mm (0.027 inch). The bore of the upper punch was next fitted onto the pin and advanced downward. The assembly was then mounted on a Carver press and the granulation was compressed into the cylindrical tablet with a force of 80-85 pounds (360-380 Newtons). The tooling was next disassembled and the tablet was ejected. The resulting cylindrical osmotic engine had an outside diameter of 1.8 mm (0.071 inch) and a length of 2.2 mm (0.087 inch). The central lumen had an inside diameter of about 1.8 mm (0.071 inch). A batch of these osmotic engine tablets was compressed for use in fabricating dilators.

Next, a coating solution of the outer membrane material was prepared. First, 4.8 grams of Tecophilic™ HP-93A-100 and 4.8 grams of Tecophilic™ SP-80A-150 were weighed and placed in a 120 cc screw-capped jar having a Teflon lined cap. The Tecophilic materials were aliphatic polyether polyurethanes sold by Lubrizol Advanced Materials, Wilmington Mass. The Tecophilic grade HP-93A-100 had a Shore hardness of 83 A, a flex modulus of 2,900 psi (as measured by the methods specified in ASTM D790) an ultimate elongation in the hydrated state of 620% (as measured by the methods specified in ASTM D412), and an equilibrium moisture content of 100%. The Tecophilic grade SP-80A-150 had a Shore hardness of 70 A, an ultimate elongation in the hydrated state of 1000%, and an equilibrium moisture content of 150%. 70.4 grams of N-methyl-2-pyrrolidone (NMP; Pharmsolve™ sold by Ashland Specialty Ingredients, Wilmington, Del.) was next added to the polymers. The jar was sealed with the lid and the resulting blend was tumble mixed for 3 days at room temperature producing a faintly hazy dispersion. The sample was then warmed in a 50° C. forced air oven for 30 minutes which clarified the solution. This completed preparation of the outer membrane coating solution.

Additional parts were fabricated in preparation for assembling the dilator. 12 inch (30.5 cm) lengths of 304 stainless steel wire having a diameter of 0.38 mm (15 mils) were cut. The cut lengths of wire then annealed in a vacuum for 1 hour at 955° C. (1750° F.). The annealed wires were then passivated in nitric acid (according to the procedures specified in ASTM-A967-05). These annealed and now malleable wires were used to fabricate the central spine of the malleable dilator.

Membrane tube stock comprising Tecophilic HP-93A-100 was extruded into four sizes. One size of tubing was extruded to an outside/inside diameter of 77 mils/38 mils (1.96 mm/0.97 mm). A second size of tubing was extruded to an outside/inside diameter of 34 mils/26 mils (0.86 mm/0.66 mm). These two sets of extruded tubing were later cut to lengths and used to fabricate the flexible spacers and end caps. A third tube was extruded to an outside/inside diameter of 25 mils/16 mils (0.63 mm/0.41 mm). This tube was later cut to a length to form the inner membrane. A fourth tube was extruded to an outside/inside diameter of 116 mils/110 mils (2.95 mm/2.82 mm). This tube was later cut to a length to form the outer membrane layer.

Handles were also pre-fabricated for later use in fabricating the dilator. The handle included a tube and a set screw. The length of the tubular handle was 2.54 cm (1.0 inch). This tube had an outside/inside diameter of 3.25 mm (0.128 inch)/0.41 mm (0.016 inch). The set screw was located 2.2 mm (0.087 inch) from the end of the handle. The handle was composed of polyetheretherketone (PEEK).

Assembly and fabrication of the dilator with a double outer membrane was next initiated. First, a 90 mm (3.5 inches) length of the annealed 15 mil (0.38 mm) wire prepared above was roll cut with a razor. Cut ends of the wire were de-burred with a metal file. A handle was slipped onto the proximal end of the wire and the set screw was tightened gently. Next, a frustum molding element comprising 303 stainless steel was slipped onto the wire such that it abutted the PEEK handle. Dimensions of the frustum molding elements were outside/outside diameters of 2.1 mm (0.083 inch)/1.1 mm (0.043 inch), length of 6.3 mm (0.248 inch), and inside diameter of 0.41 mm (0.016 inch). Next, a 2.6 mm (0.850 inch) length of od/id (outside diameter/inside diameter) 25 mils/16 mils (0.63 mm/0.41 mm) extruded inner membrane tube stock was cut to length with a razor. The cut length of inner membrane tube was wrapped in a paper towel moistened with de-ionized water and hydrated for 3 minutes at room temperature to swell it. The hydrated and now swollen inner membrane was next slipped onto the wire such that the inner membrane tube abutted the proximal frustum element. While air drying at room temperature, the expanded length of inner membrane was manually massaged down to the original 2.6 mm cut length. Next, the assembly was dried in a 50° C. forced air oven for 6 minutes. This assembly of parts comprised the malleable spine of the malleable dilator and the inner membrane of the dilator.

Next, 2.0 mm (0.079 inch) lengths of outside/inside diameter 77 mils/38 mils (2.0 mm/1.0 mm) tubing and were cut with a razor. 2.0 mm (0.079 inch) lengths of outside/inside diameter 34 mils/26 mils (0.86 mm/0.66 mm) were also cut with a razor. A 2.0 mm length of the outside/inside diameter 34 mils/26 mils (0.86 mm/0.66 mm) tubing was hydrated for 3 minutes in de-ionized water to swell it. The swollen length of tubing was removed from the water, patted dry with a paper towel, and then threaded over the inner membrane on the wire such that the hydrated tube abutted the proximal frustum element. This assembly was then dried in a 50° C. forced air oven to dry the 2.0 mm length of 34 mils/26 mils (0.86 mm/0.66 mm) tubing. Next, a 2.0 mm length of 77 mils/38 mils (2.0 mm/1.0 mm) tubing was threaded onto the assembly such it was positioned over the now dried 2.0 mm length 34 mils/26 mils (0.86 mm/0.66 mm) tubing and such that it also abutted the proximal frustum element. This bilayer tube assembly formed the precursor of the proximal end cap. Next, a cylindrical osmotic engine outside diameter of 1.8 mm (0.071 inch) and a length of 2.2 mm (0.087 inch) fabricated as previously described was slipped onto the inner membrane such it abutted this proximal end cap precursor assembly.

Next, 1.4 mm (0.055 inch) lengths of outside/inside diameter 77 mils/38 mils (2.0 mm/1.0 mm) tubing and were cut with a razor. 1.4 mm (0.055 inch) lengths of outside/inside diameter 34 mils/26 mils (0.86 mm/0.66 mm) were also cut with a razor. A 1.4 mm (0.055 inch) section of od/id 34 mils/26 mils (0.86 mm/0.66 mm) was immersed in de-ionized water for 3 minutes to hydrate it and swell it. The hydrated section was then patted dry with a paper towel and slipped onto the inner membrane such that it abutted the installed osmotic engine. The assembly was then dried in 50° C. forced air for 6 minutes. A 1.4 mm length of od/id 77 mils/38 mils (2.0 mm/1.0 mm) tubing was next slipped over the inner membrane and positioned over the now dried 1.4 mm length of od/id 34 mils/26 mils (0.86 mm/0.66 mm) such that it also abutted the osmotic engine. This bilayer construction of 1.4 mm long tubes inserted one into the other comprised the precursor of the flexible spacer of the dilator. Next, a second osmotic engine was threaded onto the inner membrane such that it abutted the now formed flexible spacer precursor.

This assembly procedure comprising the process of hydrating the 1.4 mm lengths of od/id 34 mils/26 mils (0.86 mm/0.66 mm) tubing, installing the hydrated tubing onto the inner membrane such that it abutted an osmotic engine, drying the 1.4 mm lengths of od/id 34 mils/26 mils (0.86 mm/0.66 mm) tubing, then installing the 1.4 mm length of 77 mils/38 mils (2.0 mm/1.0 mm) tubing over the od/id 34 mils/26 mils (0.86 mm/0.66 mm) tubing to form a precursor of a flexible spacer, and then slipping on an osmotic engine over the inner membrane such that it abutted the precursor of a flexible spacer was repeated three more times. The resulting assembly then comprised one end cap precursor, five osmotic engines, and four flexible spacer precursors. Finally, end cap precursor was formed on the distal end by using the same procedures and components as described to form the proximal end cap precursor.

Next, a frustum molding element having outside/outside diameters of 2.1 mm (0.083 inch)/1.1 mm (0.043 inch), length of 6.3 mm (0.248 inch), length of 6.3 mm (0.248 inch), and inside diameter of 0.41 mm (0.016 inch) was threaded onto the wire such that it abutted the distal end cap precursor. A 30 mm (1.18 inch) length of heat shrink tubing was next cut with a razor. The inside diameter of the heat shrink tubing was 1.42 mm (0.056 inch). The heat shrink tubing was composed of fluoropolymer (part number 0000053571 sold by Zeus Polymer Extrusions, Orangeburg, S.C.). The heat shrink tubing was next slipped over the assembly such that it was centered over the assembly of osmotic engines with ends of this tubing overlapping the frustum elements equally at each end. Next, another PEEK handle with set screw as described above was slipped onto the distal end of the wire such that it abutted the distal frustum element. The set screw was then tightened gently.

The assembly was then processed using a split die bonder, model 220-B sold by Beahm Designs, Inc., Los Gatos, Calif. The dilator assembly was rolled over an aluminum block heated to 199° C. (390° F.) to shrink the heat shrink tubing such that it made intimate contact with the engines, end cap precursors, and spacer precursors. The shrunken heat shrink tubing wrapped around these components and formed a continuous seal on the wide diameter of the frustum elements. The rolling proceeded from proximal to distal ends. Next, the proximal end cap precursor was melted and thermally bonded to the inner membrane using a fin die. The inside diameter of the fin die in the closed configuration was 3.81 mm (0.150 inch) and the width of the fin was 1 mm (0.039 inch). Thermal bonding was done at an air pressure of 60 pounds per square inch (4 atm) and at a temperature of 199° C. (390° F.) for a duration of 90 seconds. The assembly was cooled to room temperature. Then, the distal end cap precursor was melted and bonded to the inner membrane using these same conditions. Next, the flexible spacer precursors were melted and bonded to the inner membrane. Thermal bonding conditions were the same as described for thermal bonding the end caps except that a duration of 65 seconds was used for the spacers. The sequence of bonding the spacers was spacer 1, then spacer 4, then spacer 2, and then spacer 3 where location of the spacer was numbered starting from the proximal end. This sequence of bonding provided a more even thermal exposure than sequentially bonding positions 1, 2, 3, and then 4. No cooling cycle was used during the bonding of the spacers. During this bonding process, the bilayer tube precursor components melted and flowed. Therefore, the flat faces of spacers and end caps that had been in direct contact with the osmotic engines now assumed a concave configuration to match the convex configuration of the tablets as the molten polymer conformed to the curved faces of the tablets. Next, a small cut was made on the end of the heat shrink tubing. The exposed cut end was grabbed with pliers and the tubing was peeled off in a spiral pattern. This completed the thermal formation of the end caps and spacers and completed bonding of these end caps and spacers to the inner membrane.

Next, the outer elastic semipermeable membrane was installed and bonded to the now formed end caps and now formed spacers. First, a 21 mm length of tubular outer membrane 1 was cut from od/id of 116 mils/110 mils (2.95 mm/2.82 mm) tube stock. A single slit was next cut with a razor along the length of the cut tube. The set screw of the distal PEEK handle loosened and the handle was removed. Then, the slit outer membrane 1 was slipped over and wrapped around the circumference of the engines, spacers, and end caps such that the end of the slit tube slightly overlapped the proximal and distal frustum elements. The lengthwise cut edges formed a slight overlap of about 2 mm (0.079 inch). Excess membrane material overlapping the frustum elements was then trimmed off with a razor and discarded. Next, a 30 mm (1.18 inch) length of heat shrink tubing was cut with a razor. The inside diameter of the heat shrink tubing was 3.18 mm (0.125 inch). The heat shrink tubing was composed of fluoropolymer (part number 0000037950 sold by Zeus Polymer Extrusions, Orangeburg, S.C.). The distal PEEK handle was replaced on the wire such that it abutted the distal frustum element and the set screw was tightened gently.

The dilator assembly was then rolled over an aluminum block of the thermal welder heated to 199° C. (390° F.) to shrink the heat shrink tubing. The shrunken heat shrink tubing made intimate contact with the wrapped outer membrane. The rolling proceeded from proximal to distal ends and proceeded until the outer membrane was melted. Next, the assembly was thermally bonded for 30 seconds on the thermal welder using a channel die at 60 psi (4 atm) pressure and 199° C. (390° F.). The channel die when closed had an inside diameter of 4.57 mm (0.180 inch) and a length of 20.9 mm/(0.823 inch). A small cut was made with a razor on an end of the heat shrink tubing which tubing was peeled off in a spiral pattern. A small amount of flash, up to about 2 mm, present on the frustum elements was trimmed off with a razor and discarded. This completed full encapsulation of the five osmotic engines within the Tecophilic HP-93A-100 membrane system. Therefore, each osmotic engine resided within an individual chamber bounded by a continuous phase of the Tecophilic polymer.

Next, the outer membrane coating layer was applied over the resulting assembly by a dip coating process. The now thermally bonded dilator was immersed in the membrane coating solution prepared earlier in this example. The dilator was immersed proximal end down to a depth midway up to the distal molding cone and then slowly lifted out of the solution over duration of about 5-10 seconds. The wet dilator was then hung vertically in a current of room temperature air flowing at about 100 ft³/min (2.8 m³/min) and dried for 20 hours. At the end of this drying cycle, the dried drip that had formed on the bottom of the proximal end was trimmed off and discarded. The dilator was then inverted 180° and dipped into the coating solution distal end down to a depth midway up to the proximal frustum element. The dilator was again dried for 20 hours in a current of room temperature air. This dip coating process was performed two more cycles such that a total of four dip coatings and four drying cycles had been completed. After the fourth and final drying cycle, the circumference of now formed outer membrane was cut at each end with a razor such that it spanned the distance between the frustum elements. Next, the set screws of the PEEK handles were loosened, the handles were removed, and the 2.1 mm outside diameter frustum elements were removed and replaced with fresh frustum elements. Dimensions of the fresh frustum elements were od/od 2.6 mm (0.102 inch)/(2.6 mm/(0.080 inch), length of 6.2 mm (0.245 inch) and id of 0.51 mm (0.020 inch). The 2.6 mm outside diameter (OD) frustum elements had been machined from 303 grade stainless steel. Next, a 30 mm (1.18 inch) length of fluoropolymer heat shrink tubing was cut with a razor. The inside diameter of the heat shrink tubing was 3.18 (0.125 inch). The cut length of heat shrink tubing was slipped over the dilator such that it was centered over the dilator and overlapped the frustum elements by about the same amount at each end. The PEEK handles were next replaced and the set screws were tightened. The heat shrink tubing was then shrunk by rolling over an aluminum block on the thermal bonder that was heated to 199° C. (390° F.). The rolling continued until the outer membrane layer was molten. The assembly was then cooled to room temperature. Next, an outer membrane layer was thermally bonded to both end caps using a fin die having an inside diameter when closed of 3.81 mm (0.150 inch) and width of 1 mm (0.039 inch) at an air pressure of 60 pounds per square inch (4 atm) at 199° C. (390° F.) for 115 seconds. The dilator was cooled to room temperature between bonding the proximal end cap and the distal end cap. After cooling to room temperature, a small slit was cut in the heat shrink tubing. Then, the exposed end of heat shrink tubing was grabbed with pliers and peeled off in a spiral pattern. The distance from the proximal end cap to the distal end cap was measured at 19.4 mm. The working length of the dilator, representing the span from the distal osmotic engine to the proximal osmotic engine, was measured at 15.7 mm. The diameter of the dilator was measured with digital calipers at 2.6 mm. This completed the osmotic working element of the osmotic frontal sinus dilator. Malleability of the dilator was 11.0 Newtons.

The resulting device was tested in vitro to monitor dilation as a function of time. The test was conducted in a USP Type 2 paddle tester in a volume of 500 cm³ of de-ionized water maintained at a constant temperature of 37° C. and stirred at 50 revolutions per minute. Diameter was monitored versus time with a digital caliper. The dilator expanded from an initial diameter of 2.6 mm to 3.7 mm in 30 minutes, to 4.6 mm by 60 minutes, and to 4.7 mm by 90 minutes. The engines of the dilator swelled uniformly.

### Example 10

A dilator was fabricated according to the procedures and materials described in Example 9 up to the point of in vitro testing. Set screws of the PEEK handles were loosened and the handles were removed. Then, the 2.6 mm frustum elements were removed. This provided the bare osmotic swelling element with five engines, two end caps, and four spacers and about 30 mm of 15 mil (0.038 mm) diameter annealed wire free at the proximal and distal ends. Next, a nose cone was slipped onto the distal end such that it abutted the distal end cap. The nose cone had been formed by injection molding polyether block amide copolymer (PEBAX 55). The outside diameter of the cone was 2.8 mm (0.110 inch) and the length of the cone was 4.6 mm (0.181 inch). The inside diameter of the nose cone was 0.48 mm (0.019 inch). Next, a malleable sleeve was slipped onto the 15 mil wire such that it abutted the nose cone. The outside and inside diameter of the sleeve was 0.64 mm (0.025 inch) and 0.51 mm (0.020 inch), respectively. The length of the sleeve was 5.4 mm (0.213 inch). The sleeve was composed of 304 stainless steel that had been annealed at 955° C. (1750° F.). Loctite 4011 cyanoacrylate adhesive was next applied to the 15 mil (0.038 mm) wire to bond the sleeve to the annealed wire. Next, the loose end of the wire adjacent to the sleeve was formed into a loop by wrapping the annealed wire twice around a round gauge pin. The round gauge pin had an outside diameter of 0.91 mm (0.036 inch). The remaining length of wire was grabbed with pliers and then wrapped around the sleeve with two turns. The excess wire was clipped off flush to the sleeve. This process formed a guidewire loop and anchored the distal end such that it could not expand axially during device operation. The outside diameter of the loop was 1.7 mm.

Next, a washer was threaded onto the 15 mil wire on the proximal end such that it abutted the proximal end cap. The dimensions of the washer were outside/inside diameter of 2.7 mm (0.106 inch)/0.41 mm (0.016 inch). Width of the washer was 1.0 mm (0.039 inch). The washer had been machined from PEEK. A drop of Loctite 4011 cyanoacrylate adhesive was placed on the wire adjacent to the washer to anchor the washer. Then, a 12 inch (30.5 cm) length of Kevlar thread was tied onto the wire adjacent to the washer with a double square knot. The Kevlar thread was 23 pound (102 Newton) test and size 69 as supplied by the ThreadExchange.com. Excess thread was trimmed with a razor. Next, a smaller washer was inserted onto the wire such that it abutted the knot. The dimensions of the smaller washer were outside/inside diameter of 2.0 mm (0.080 inch)/0.64 mm (0.025 inch). Width of the smaller washer was 1.3 mm (0.050 inch). The smaller washer had been machined of PEEK. Next, the free end of the 15 mil (0.038 mm) wire on the proximal end was bent with pliers over the smaller washer in a 90 degree bend. The wire was then wrapped twice around the wire and positioned adjacent to the washer. Excess wire was trimmed and the cut end was formed into the recess between the smaller washer and the larger washer. Finally, Loctite 4011 cyanoacrylate adhesive was placed onto the tether and small washer to secure the washers and tether to the wire. This completed fabrication of the frontal sinus dilator.

In certain embodiments, the healthcare provider (e.g., surgeon) first performs a computerized tomography (CT) scan of the patient to identify and characterize anatomical features of the stenotic frontal sinus. This information facilitates placement of the device near the opening of the frontal sinus recess under endoscopic examination. In practice, a guide wire with light source typically with an outside diameter of about 0.86 mm (0.034 inch) is first threaded through the loop present on the distal end of the device. The assembled lighted guide wire and device pair are then inserted into the nasal cavity of the patient and advanced to the region of the frontal sinus recess. In certain embodiments, the guide wire is first advanced and used to probe and identify the location of the frontal sinus. The desired location is identified when the light source enters the frontal sinus cavity and light from the light source transilluminates the forehead of the patient in a darken room. Next, under continued endoscopic visualization, the dilator is advanced by sliding the loop of the dilator along the guide wire until it reaches the opening of the frontal sinus recess. The dilator is next advanced a few millimeters into the opening and then the guide wire is retracted. The removal of the guide wire provides easier access to the narrow, stenotic, slit-like opening. The dilator is next advanced about 13 to 17 mm such that the osmotic engines are nested within the mucosal tissue of the frontal recess. Because the loop is fabricated with malleable wire, it can bend, conform, contort, and even collapse in order for the dilator to enter the space and be seated properly within the fontal recess. The dilator is then left in place for a duration of 60 to 90 minutes during which time it imbibes water from the mucosal tissue and swells radially. Force of the radial expansion remodels and enlarges tissues of the recess and dilates it. The dilator is finally removed from the recess by the healthcare provider (e.g., surgeon) either by using grabbing forceps or by pulling the tether. Once removed, the dilated recess allows ventilation and drainage to promote sinus health.

### Example 11

A dilator was fabricated according to the procedures and materials described in Example 10 except that the configuration of the distal end is different. The configuration of this dilator is shown in FIG. 58. A malleable sleeve was slipped onto the 15 mil (0.38 mm) wire such that it abutted the nose cone. Outside and inside diameters of the sleeve were 0.64 mm (0.025 inch) and 0.51 mm (0.020 inch), respectively. Length of the sleeve was 2.0 mm (0.079 inch). The sleeve was composed of 304 stainless steel that had been annealed at 955° C. (1750° F.). Loctite 4011 cyanoacrylate adhesive was next applied to the 15 mil (0.38 mm) wire to bond the sleeve to the annealed wire. Next, the free end of the wire adjacent to the sleeve was cut to a length of 20 mm as measured from the sleeve. A bead having a diameter of 0.93 mm (0.037 inch) was then formed on the cut end using an arc welding process in argon gas. The bead was formed with a THERM-X (model 300) gas tungsten welder by feeding the wire into a 0.016 ID copper tube that that had a chamfer of approximately 0.032 inches (0.8 mm) and welding for duration of 0.5 seconds in an atmosphere of 10-15 pounds per square inch (0.7 to 1 atm) of argon gas. Then, the free end of the 20 mm length of wire was formed into coil by wrapping the annealed wire three times around a round gauge pin. The round gauge pin had an outside diameter of 0.91 mm (0.036 inch). This process formed a malleable coil on the distal end of the dilator which coil is used during placement to guide the lighted guide wire.

Prior to placing the dilator into the nasal cavity, the lighted guide wire is threaded into the coil. Then, the coil and guide wire pair under endoscopic visualization are placed into the nasal cavity and advanced to the entrance of the frontal sinus recess. The lighted guide wire is then inserted into the recess in advance of the distal coil of the dilator. Correct placement of the lighted guide wire within the frontal sinus is confirmed by transillumination of the forehead by light emanating through tissues of the forehead from within the cavity of the frontal sinus. Once placement is confirmed, the distal coil of the dilator is advanced a few millimeters into the entrance of the frontal recess. The guide wire is next retracted out of the entrance of the recess. Then, the dilator is advanced into the frontal recess until it is fully seated within the frontal sinus recess. The dilator is then left in place for a duration of 60 to 90 minutes during which time it imbibes water from the mucosal tissue and swells radially. The force of the radial expansion dilates and remodels the recess. The dilator is finally removed from the recess by the healthcare provider (e.g., surgeon) either by using grabbing forceps or by pulling the tether. Once removed, the dilated recess allows ventilation and drainage to promote sinus health.

As can be appreciated from the disclosure provided above, the present disclosure has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

The preceding merely illustrates the principles of the disclosure. All statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, e.g., any elements developed that perform the same function, regardless of structure. The scope of the present disclosure, therefore, is not intended to be limited to the exemplary embodiments shown and described herein. Rather, the scope and spirit of present disclosure is embodied by the appended claims.

1. A dilator for dilating a stenotic recess of a frontal sinus in a subject, the dilator comprising a self-expanding member configured to radially expand from a non-expanded configuration to an expanded configuration, the non-expanded configuration being sized to be positioned within the stenotic recess, the dilator comprising a radially-expandable working length, said working length comprising (i) a plurality of non-bendable segments, and (ii) one or more bendable segments positioned between adjacent non-bendable segments, the dilator having a malleability of 30 Newtons or less.

2. The dilator of claim 1, wherein the dilator has a malleability of 5 Newtons or more.

3. The dilator of claim 1, wherein the dilator has a malleability of 10 to 20 Newtons.

4. The dilator of claim 1, wherein the dilator has a malleability of 15 Newtons or more.

5. The dilator of claim 1, wherein the dilator has a malleability of 20 to 25 Newtons.

6. The dilator of claim 1, comprising: (i) a malleable axial member having a plurality of solid osmotic tablets disposed thereon and spaced apart from one another; and (ii) an elastic semipermeable membrane disposed over the tablets.

7. The dilator of claim 6, wherein the malleable axial member is selected from (i) a rod, (ii) a tube, (iii) a coil, and (iv) a wire.

8. The dilator of claim 6, wherein the axial member is radially non-compressible and composed of a malleable metal or polymer.

9. The dilator of claim 8, wherein the malleable metal is selected from aluminum, silver, gold, platinum, titanium, copper and annealed stainless steel.

10. The dilator of claim 8, wherein the malleable polymer is selected from cross-linked polybutadiene, polyvinyl acetate, ethylene vinyl acetate and ethyl acrylate methyl methacrylate copolymer.

11. The dilator of claim 6, including elastic spacers disposed on the axial member, the spacers being positioned between adjacent osmotic tablets.

12. The dilator of claim 1, having from 3 to 8 of the bendable segments and from 2 to 7 of the non-bendable segments.

13. The dilator of claim 1, having from 5 to 7 of the bendable segments and from 4 to 6 of the non-bendable segments.

14. The dilator of claim 1, wherein the working length is 12 to 24 mm.

15. The dilator of claim 1, wherein the working length is 13 to 16 mm.

16. The dilator of claim 1, wherein the dilator has an overall length that is greater than the working length.

17. The dilator of claim 1, wherein each of the bendable segments has a length of 0.5 mm or more.

18. The dilator of claim 1, wherein each of the bendable segments has a length of 1 to 1.5 mm.

19. The dilator of claim 1, wherein each of the non-bendable segments has a length of 4 mm or less.

20. The dilator of claim 1, wherein each of the non-bendable segments has a length of 2 to 3 mm.

21. The dilator of claim 1, comprising an elongated flexible portion extending from a distal end of the dilator.

22. The dilator of claim 21, wherein the elongated flexible portion comprises a spring.

23. A kit comprising:
a dilator according to claim 1; and
a device for inserting the dilator into a frontal sinus recess.

24. The kit of claim 23, including two or more dilators.

25. A device for inserting an osmotically-driven sinus dilator into a stenotic opening of a paranasal sinus in a subject, the dilator being flexible and/or bendable along its length, the device comprising:
a handle including a guidewire actuator and a dilator actuator;
a tube having a proximal end coupled to the handle, a distal end adapted to be positioned adjacent the stenotic sinus opening, and an interior cavity extending from the proximal end to the distal end, the interior cavity being sized to fit the dilator therein;
a guidewire extending through the interior cavity, the guidewire being displaceable through or adjacent to the dilator upon activation of the guidewire actuator;
an arm extending within the interior cavity of the tube, the arm being displaceable upon activation of the dilator actuator for deploying the dilator from the interior cavity.

26. The device of claim 25, wherein the dilator comprises an element configured to slide over the guidewire.

27. The device of claim 26, wherein the element is an axial passageway through the dilator, the guidewire extending through the axial passageway when the dilator is positioned in the tube.

28. The device of claim 26, wherein the element is a loop attached to a distal end of the dilator.

29. The device of claim 25, wherein the distal end of the tube is angled.

30. The device of claim 29, wherein the sinus opening is a frontal sinus recess and the angle is from 50° to 90°.

31. The device of claim 30, wherein the angle is 70°.

32. The device of claim 29, wherein the sinus opening is a maxillary sinus opening and the angle is from 90° to 140°.

33. The device of claim 32, wherein the angle is 110°.

34. The device of claim 29, wherein the sinus opening is a sphenoid sinus opening and the angle is from 1° to 30°.

35. The device of claim 34, wherein the angle is 15°.

36. The device of claim 25, wherein activation of the guidewire actuator causes the guidewire to be displaced distally beyond the distal end of the tube.

37. The device of claim 25, wherein activation of the dilator actuator causes the arm to be displaced distally to push the dilator out of the distal end of the tube.

38. The device of claim 25, wherein the dilator has a radially extending anchor and the distal end of the tube has a slot that accommodates the anchor while the dilator is positioned in the interior cavity.

39. The device of claim 25, wherein the arm has a bore through which the guidewire extends.

40. The device of claim 25, including a light source or a light-transmitting conduit for illuminating the nasal cavity and/or the sinus opening during dilator insertion.

41. The device of claim 40, wherein the guidewire is the light transmitting conduit.

42. An assembly comprising the device of claim 25 and an osmotically-driven sinus dilator positioned in the interior cavity.

43. A method of dilating a stenotic opening of a paranasal sinus in a subject, the method comprising:
placing an osmotically-driven sinus dilator in the interior cavity of the device of claim 25;
inserting the tube into a nasal cavity of the subject;
positioning the distal end of the tube adjacent the stenotic opening;
activating the guidewire actuator causing the guidewire to extend beyond the distal end of the tube and into and/or through the sinus opening;
activating the dilator actuator causing the dilator to be displaced distally along the extended guidewire and into the sinus opening; and
removing the device from the nasal cavity of the subject while leaving the dilator in place in the stenotic opening.

44. The method of claim 43, wherein the dilator comprises an element configured to slide over the guidewire, and activating the dilator actuator causes the dilator to slide over the extended guidewire.

45. The method of claim 43, wherein activating the dilator actuator causes the dilator to be displaced distally adjacent to the extended guidewire.

46. The method of claim 43, wherein the dilator dilates by way of osmosis after the device is removed from the nasal cavity.

47. The method of claim 43, comprising removing the dilator from the sinus opening after the dilator has dilated.

48. The method of claim 43, wherein the sinus opening is selected from a maxillary sinus opening, a frontal sinus recess and a sphenoid sinus opening.

49. The method of claim 43, comprising illuminating the nasal cavity and/or the sinus opening during placement of the dilator into the stenotic opening.
